(19) United States
(12) Patent Application Publication
(10) Pub. No.: US 2026/0217660 A1
(43) Pub. Date: Jul. 30, 2026

(54) INHIBITORS OF ACTIVIN RECEPTOR TYPE I (ACVRI) AND ACTIVIN RECEPTOR LIKE TYPE I (ACVRLI), AND METHODS USING SAME

(71) Applicant: Baylor College of Medicine, Houston, TX (US)

(72) Inventors: Martin M. Matzuk, Houston, TX (US); Ravikumar Jimmidi, Houston, TX (US); Diana Monsivais, Houston, TX (US); Stephen Palmer, Houston, TX (US); Damian Young, Houston, TX (US); Kurt M. Bohren, Houston, TX (US); Feng Li, Houston, TX (US); Jian-Yuan Li, Houston, TX (US); Zhi Tan, Houston, TX (US)

(21) Appl. No.: 19/148,738

(22) PCT Filed: Jan. 17, 2024
(86) PCT No.: PCT/US2024/011839

§ 371 Date: Jul. 17, 2025

Related U.S. Application Data
(60) Provisional application No. 63/439,479, filed on Jan. 17, 2023.

(51) Int. Cl. C07D 235/18; A61K 47/68; C07D 401/04; C07D 401/12; C07D 405/12; C07D 471/04

(52) U.S. Cl. CPC C07D 235/18; A61K 47/6803; A61K 47/6871; C07D 401/04; C07D 401/12; C07D 405/12; C07D 471/04

(57) ABSTRACT

In one aspect, the present disclosure relates to compounds of Formula (I), which inhibit Activin A receptor type 1 (ACVR1 or ALK2) and/or mutants thereof, and pharmaceutical compositions thereof, and/or which inhibit Activin A receptor like type 1 (ACVRL1 or ALK1) and/or mutants thereof, and pharmaceutical compositions thereof. In another aspect, the present disclosure provides a method of treating, preventing, and/or ameliorating a disease and/or disorder in a subject, the method comprising administering to the subject at least one compound and/or pharmaceutical composition of the present disclosure. In certain embodiments, the disease and/or disorder is characterized by one or more ACVR1 and/or ACVRL1 activating mutations and/or overactive ACVR1 and/or overactive ACVRL1. In another aspect, the present disclosure provides a method of inhibiting wild-type or mutant ACVR1 and/or ACVRL1 in a subject, the method comprising administering to the subject at least one compound and/or pharmaceutical composition of the present disclosure.

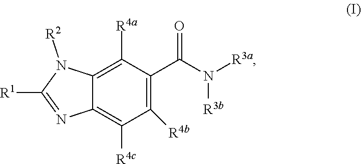

### CROSS-REFERENCE TO RELATED APPLICATION

This application claims priority under 35 U.S.C. § 119 (e) to U.S. Provisional Patent Application No. 63/439,479 entitled “INHIBITORS OF ACTIVIN RECEPTOR TYPE 1 (ACVR1) AND ACTIVIN RECEPTOR LIKE TYPE 1 (ACVRL1), AND METHODS USING SAME,” filed Jan. 17, 2023, the disclosure of which is incorporated herein by reference in its entirety.

### STATEMENT REGARDING FEDERALLY SPONSORED RESEARCH OR DEVELOPMENT

This invention was made with government support under HD032067 awarded by the National Institutes of Health. The government has certain rights in the invention.

### BACKGROUND

Activin receptor type 1 (ACVR1; ALK2) and Activin receptor like type 1 (ACVRL1; ALK1) are transforming growth factor beta (TGFβ) family cell surface signaling receptors that integrate extracellular concentrations of activin and bone morphogenic proteins (BMPs) into SMAD signaling complexes. The predominant signaling pathway stimulated by ACVR1 and ACVRL1 involves SMAD1/SMAD5. Several activating mutations in ACVR1 cause Fibrodysplasia Ossificans Progressive (FOP), Diffuse Intrinsic Pontine Glioma (DIPG), and ependymoma. The ACVR1 R206H mutation is also present in a subset of endometrial tumors, melanomas, non-small lung cancers, and colorectal cancers, and ACVR1 expression is elevated in pancreatic cancer.

Mutations in the ACVR1 receptor cause a shift in the ligand specificity of ACVR1 and enable stimulation of SMAD1/5 by a broader group of TGFβ family proteins (e.g., Activin A). Because of its critical role in the control of iron metabolism, inhibition of ACVR1 may also be beneficial for treatment of atherosclerosis, severe iron deficiency anemia, and other causes of anemia, including anemia of chronic disease caused by inflammation. Thus, inhibition of the kinase activity of ACVR1 is a promising approach for the treatment of FOP, DIPG, ependymomas, and endometrial cancer, as well as other cancers, atherosclerosis, iron deficiency anemia, tumor vasculogenesis, and Sjögren's syndrome.

Beyond diseases that carry genetic mutations in ACVR1 or ACVRL1, there are several benign indications in which an ACVR1 inhibitor may be useful with appropriate cell targeting. For example, an ACVR1 inhibitor can be targeted to ovarian follicular cells that express AMHR2 through antibody-directed delivery of an ACVR1 inhibitor. Similar approaches can be used to direct delivery of an ACVR1 inhibitor to other cells in the body, wherein targeted delivery is required, rather than for treatment of cells with ACVR1 genetic mutations. Examples of successful ADCs include gemtuzumab ozogamicin and brentuximab vedotin (reviewed by Birrer et al. 2019, JNCI J Natl Cancer Inst (2019) 11 (6): djz035).

Current inhibitors of ACVR1 (e.g., LDN-212854, BLU-782, and LDN-193189) have off-target effects on other related BMP pathway receptors (i.e., ALK1, ALK3, ALK6), TGFβ/activin pathway receptors (i.e., ALK4, ALK5, ALK7), and other kinases. Further, current inhibitors of ALK4, ALK5, and/or ALK7 do not inhibit ACVR1 (e.g., SB431542). Additionally, the discovery of ACVR1-specific inhibitors is important for chronic dosing, as ALK5 inhibition can result in heart valve lesions, and ALK4, ALK5, and/or ALK7 inhibition could exacerbate systemic inflammation.

There are currently no highly specific inhibitors available for the ACVR1 proteins involved in inter alia FOP, DIPG, and pancreatic cancer (which is a very lethal cancer), and accordingly, identification of any therapeutic compounds that can extend patient survival would be considered a major breakthrough in the field.

Thus, there is a need in the art for selective inhibitors of ACVR1 and/or mutants thereof, and methods of use thereof. The present disclosure addresses this need.

### BRIEF SUMMARY

The disclosure provides in one aspect a compound of formula (I):

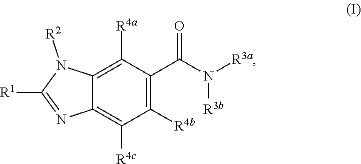

wherein R¹, R², R^{3a}, R^{3b}, R^{4a}, R^{4b}, and R^{4c} are defined elsewhere herein, or a salt, solvate, stereoisomer, or isotopologue thereof.

The disclosure provides in one aspect an antibody-drug conjugate (ADC) comprising at least one compound of the disclosure and an antibody, wherein the compound is covalently conjugated to the antibody.

The disclosure provides in one aspect a pharmaceutical composition comprising at least one compound of the disclosure and/or the antibody-drug conjugate of the disclosure and a pharmaceutically acceptable carrier.

The disclosure provides in one aspect a method of treating, preventing, and/or ameliorating a disease or disorder in a subject in need thereof, the method comprising administering to the subject a therapeutically effective amount of at least one compound of the disclosure, the antibody-drug conjugate (ADC) of the disclosure, and/or the pharmaceutical composition of the disclosure.

The disclosure provides in one aspect a method of inhibiting and/or reducing activity of ACVR1 in a subject in need thereof, the method comprising administering to the subject a therapeutically effective amount of at least one compound of the disclosure, the antibody-drug conjugate (ADC) of the disclosure, and/or the pharmaceutical composition of the disclosure.

The disclosure provides in one aspect a method of inhibiting and/or reducing activity of ACVRL1 in a subject in need thereof, the method comprising administering to the subject a therapeutically effective amount of at least one compound of the disclosure, the antibody-drug conjugate (ADC) of the disclosure, and/or the pharmaceutical composition of the disclosure.

### BRIEF DESCRIPTION OF THE FIGURES

The drawings illustrate generally, by way of example, but not by way of limitation, various embodiments of the present application.

FIG. 1 provides a schematic depicting the TGFβ family signaling pathway, where BMP ligands signal via a Type 1 and Type 2 cell surface receptor complex. The Type 1 receptors depicted in FIG. 1 are ACVR1 (ALK2) and ACVRL1 (ALK1). Upon ligand binding, the intracellular kinase domains are phosphorylated and activate a downstream signaling cascade via the phosphorylation of SMAD1 and SMAD5 transcription factors.

FIG. 2 provides a graph depicting the enrichment profile of a DNA-encoded library against ACVR1 at 0.3 μM. The selected data show enrichment of the same building block 1 component (i.e., top compound in box) and building block 3 component (i.e., bottom compound in box) with various building block 2 components (i.e., middle compound in box). The enrichment of each hit series was shown as count/z-score at 0.06 μM and 0.3 μM. Building block 1 is attached to the DNA and building block 3 is farthest from the DNA. All hits in this series are competitive with a kinase inhibitor.

FIGS. 3A-3B depict a representation of KINOMEscan (Eurofins) binding data of CDD-2342 across the human kinome phylogenetic tree (FIG. 3A) and ThermoFisher Lantha kinase screen (FIG. 3B). FIG. 3A: The size of the circles (indicated with lines) are correlated with the affinity of ligand binding (i.e., larger circle, greater binding affinity). CDD-2342 is highly specific even when tested at a dose of 1 μM and has minimal off-target binding. FIG. 3B: Using the ThermoFisher Lantha kinase screening assay, kinase specificity for ACVR1>ACVRL1>>BMPR1A>BMPR1B was observed.

FIGS. 4A-4C provide graphs depicting live-cell target engagement for ACVR1 (FIG. 4A), mutant ACVR1 (R206H) (FIG. 4B), and ACVRL1 (FIG. 4C). The Promega NanoBRET target engagement system involves cell-based competitive displacement of a fluorescent NanoBRET tracer bound reversibly to a NanoLuc fusion protein. Test compounds displace the tracer dependent on their affinity for the target and reduce loss of the NanoBRET signal. Staurosporine and CDD-2342 were assayed in Promega's ACVR1, ACVR1^{R206H}, and ACVRL1 NanoBRET cell-based kinase assay system. CDD-2342 enters the cell and is a more potent nanomolar inhibitor against ACVR1 and ACVR1^{R206H} than Staurosporine, a promiscuous kinase inhibitor and a positive control in the assays provided herein.

FIG. 5 provides a bar graph depicting Activin-mediated hepcidin (HAMP) induction in HEPG2 cells. HEPG2 cells were grown in the presence of low (left bar graphs) and high dose iron levels (right bar graphs) and stimulated with Activin (Act), Tacrolimus (Tac), or BMP2. Treatment with high doses of tacrolimus displaces FKBP12, rendering the cells responsive to activin and inducing Hepcidin (HAMP) gene expression with similar levels as BMP2. This demonstrates a useful cellular system for the testing of ACVR1 inhibitors for the treatment of patients with tacrolimus-induced iron accumulation and anemia.

FIG. 6 shows 293T-BRE-R cells pre-treated with 1 μM, 5 μM, or 10 μM CDD-2342 for 30 minutes followed by stimulation with vehicle (DMSO) or 5 ng/ml BMP2. BMP2-dependent luciferase reporter activation was quantified after 6 hours using the Promega BrightGLO luciferase system on a Tecan Infinity M1000 Pro plate reader. CDD-2342 significantly inhibits BMP2-induced reporter activity in the HEK-293T cells at all tested concentrations (i.e., 1 μM, 5 μM, and 10 μM).

FIG. 7 provides a bar graph showing cytotoxicity as a function of CDD-2789 concentration in HepG2 cells. Cell viability was quantified 24 hours after administration of CDD-2789 by quantifying cellular ATP using Cell Titer GLO, wherein no cytotoxicity in HepG2 cells was observed at concentrations up to 100 μM.

FIGS. 8A-8B show a kinase selectivity profile of CDD-2789. FIG. 8A: KINOME scan analysis of CDD-2789 binding to 468 kinases assayed at 1 μM in duplicate. Compound selectivity is represented in a TREEspot kinase dendrogram view of the human kinome phylogenetic tree. FIG. 8B: IC₅₀ values of compound CDD-2789 against different kinases shown using Thermofisher Lantha Screen or ZLYTE assays.

FIG. 9 provides a graph depicting pharmacokinetic studies of CDD-2879 in mice.

FIG. 10 provides a bar graph depicting inhibition of BMP2-induced luciferase activity in 293T-BRE-reporter cells. Approximately 20,000 cells per well were plated in a 96-well plate and allowed to attach overnight. Subsequently, cells were pre-treated with vehicle (i.e., DMSO) or ACVR1 inhibitors (i.e., CDD-2340, CDD-2282, CDD-2787, CDD-2281, CDD-2315, and CDD-2789) at various concentrations (i.e., 0.2 1, 5, and 25 μM) for 30 minutes in phenol-red-free DMEM supplemented with 2 mM glutamine and 2% heat-inactivated FBS. Cells were then stimulated with vehicle or 5 ng/ml BMP2 for 6 hours. Luciferase transactivation was assessed using the Promega BrightGLO luciferase system (Promega) and the signal was quantified using a plate reader (M1000 Pro). Potent inhibition of BMP2-induced luciferase activity was observed with CDD-2340, CDD-2787, CDD-2315, and CDD-2789.

FIG. 11 depicts inhibition of phosphorylated SMAD1/5 (pSMAD1/5) in primary WT and FOP fibroblasts. Primary fibroblasts isolated from patients with fibrodysplasia ossificans progressiva (FOP GM00513) or a healthy individual (WT GM01652) were pre-treated with various concentrations (i.e., 1, 5, and 25 μM) of ACVR1 inhibitors (i.e., CDD-2340, CDD-2787, CDD-2315, and CDD-2789) or LDN-192189 (1 μM) for 30 minutes. Subsequently, the fibroblasts were treated with vehicle (i.e., DMSO) or 5 ng/mL BMP2 in the presence of the inhibitors for 45 minutes. Cell lysates were then probed with antibodies against pSMAD1/5, total SMAD5, total SMAD1, and GAPDH (i.e., loading control) using a Western blot. It was found that ACVR1 inhibitors suppressed BMP2-induced pSMAD1/5 activation in the FOP and WT fibroblasts in a dose-dependent manner.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

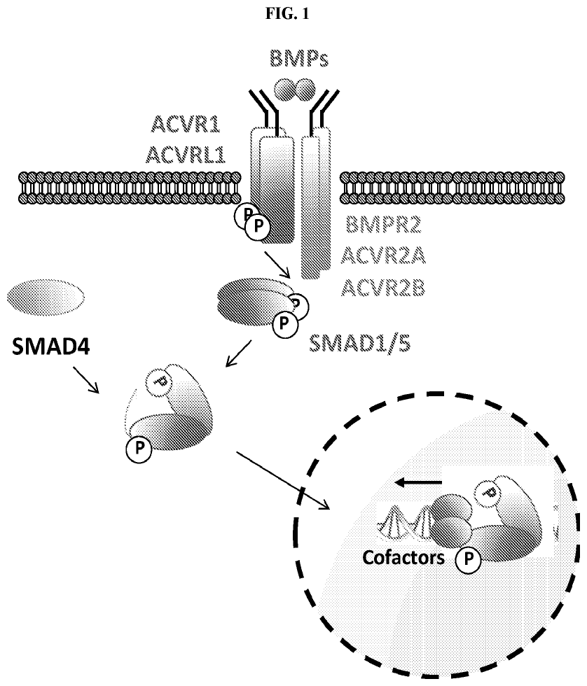

ACVR1 (ALK2 or ALK-2) and ACVRL1 (ALK1 or ALK-1) are type 1 transforming growth factor beta (TGFβ) family cell surface signaling receptors. ACVR1 and ACVRL1 are downstream of activin and Bone Morphogenic Protein (BMP) ligands and can phosphorylate SMAD1 and SMAD5 (i.e., “BMP” SMADs) when ligands bind a complex of ACVR1 and a type 2 receptor (e.g., ACVR2A, ACVR2B, AMHR2, and/or BMPR2) (FIG. 1). Several activating mutations in ACVR1 cause FOP and DIPG. The ACVR1 R206H mutation is also present in a subset of endometrial tumors, and ACVR1 expression is elevated in pancreatic cancer. Because of its critical role in the control of iron metabolism, inhibition of ACVR1 can also be beneficial for treatment of atherosclerosis, and patients with severe iron deficiency anemia and causes of anemia, among other indications.

The kinase activity of ACVR1 is a target for the treatment of FOP, DIPG, other cancers, atherosclerosis, iron deficiency anemia, tumor vasculogenesis, and Sjögren's syndrome, inter alia. Current inhibitors of ACVR1 (e.g., LDN-212854, BLU-782, LDN-193189) have off-target effects on other related BMP pathway receptors (e.g., ALK1, ALK3, ALK6), TGFβ/activin pathway receptors (e.g., ALK4, ALK5, ALK7), and other kinases. Because ALK5 inhibition can result in heart valve lesions and ALK4, ALK5, and ALK7 inhibition could cause systemic inflammation, finding ACVR1-specific inhibitors is important for chronic dosing.

Thus, in one aspect, the present disclosure relates to the use of a DNA-encoded chemistry technology (DEC-Tec) platform for the identification of small molecule inhibitors of ACVR1 and ACVRL1. The present disclosure further relates to confirmation and/or optimization of identified compounds by off-DNA synthesis and evaluation in a number of biological assays. In one aspect, the experiments disclosed herein describe the inhibitory potency, selectivity, in vivo half-life, low toxicity, and metabolic stability of compounds CDD-2342 and CDD-2789.

There are currently no highly selective inhibitors against ACVR1 and/or ACVRL1, and/or mutants thereof, and thus the present disclosure comprises a significant advancement in the development of therapeutic interventions for a number of significant diseases and/or disorders.

### Validation of ACVR1 Pathway Inhibition

ACVR1^{R206H} mutations that result in overactive BMP/SMAD1/5 signaling have been found in approximately 25% of children with diffuse intrinsic pontine glioma (DIPG) and in a small subset of patients with posterior fossa ependymomas. The same activating ACVR1^{R206H} mutations exist in patients with fibrodysplasia ossificans progressiva (FOP) and in a subset of endometrial tumors. Additional activating ALK2 mutations, such as ALK2 (G328E, G328W, and G328V) have also been identified in the tumors of patients with DIPG. Iron refractory deficiency anemia (IRIDA) is characterized by elevated liver hepcidin production caused by overactive BMP/SMAD signaling. Therefore, several diseases characterized by overactive BMP/SMAD signaling would benefit from small molecule inhibition of ACVR1 kinase activity. Beyond genetic diseases, additional human validation has been obtained from organ transplant patients receiving rapamycin, tacrolimus, or everolimus who experience a drug-induced increase in ACVR1 stimulation that contributes to elevated hepcidin production and iron-associated anemia.

Additionally, mice with a humanized conditional ACVR1^{R206H} knock-in allele have been shown to develop ectopic bone formation similar to that observed in patients with FOP. A transgenic mouse line with an activating ACVR1^{Q207D} mutation that develop heterotopic ossification and FOP-like disease are also available for testing small molecules. The mutant mice display aberrant activin A induction of SMAD1/5 signaling and ectopic bone formation. A mouse line expressing green fluorescence protein (GFP) driven by a BMP reporter element (BRE:GFP) is also available, with such mice being useful to assess the ability of novel ACVR1 inhibitors to suppress BMP-induced gene activation in various cell types and organ systems. Primary cells established from the tumors of patients with DIPG which harbor ACVR1 mutations are also available for testing the efficacy of these ACVR1 inhibitors.

The present disclosure provides data for the inhibition of ACVR1, and related proteins, utilizing a number of assays, non-limiting examples including Promega NanoBRET luciferase ACVR1, ACVR1^{R206H}, and ACVRL1 assays. In certain embodiments, the assays described herein are useful to test BMP receptor activation and/or small molecule inhibition. Further, HEK29T cells with stable expression of a BMP reporter element driving luciferase expression can be used to test inhibition of BMP-induced luciferase transactivation. Utilizing such a system, the present disclosure provides validation of the potency of various ACVR1 inhibitors. Further, BMP2- and BMP9-induction of SMAD1/5 phosphorylation (pSMAD1/5) in HEK293T and human umbilical vein endothelial cells (HUVEC) can be reliably obtained. Additionally, utilizing the HEPG2 cell system described herein, responsiveness to activin and induction of Hepcidin (HAMP) gene expression can be probed in the presence and absence of ACVR1 inhibitors, as well as the influence of tacrolimus on activin-induced hepcidin gene expression.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)—CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, epitope binding antibody fragments (such as Fab, Fab′, F(ab′)₂, and Fv fragments), single chain Fv (scFv) mutants, immunoglobulin new antigen receptor antibodies (IgNARs), which comprise single variable new antigen receptor domain antibody fragments (VNARS, or VNAR domains), unibodies, in which the hinge region has been removed, nanobodies, antibody fragments consisting of a single monomeric variable antibody domain (Ablynx), minibodies, which are engineered antibody fragments comprising an scFv linked to a CH domain (Hu et al., Cancer Res. 56:3055-3061, 1996), DuoBodies®, which are bispecific modified IgG1 antibodies that include (i) a stable hinge region that is non-permissive for Fab arm exchange in vivo and (ii) an IgG4-like CH₃ domain modified to be permissive for Fab arm exchange in vivo. (See, for example, WO2008/119353 and WO2011/131746), multispecific antibodies, such as bispecific antibodies generated from at least two intact antibodies, probodies, which are recombinant, masked monoclonal antibodies that remain inert in healthy tissue, but are activated specifically in the disease microenvironment (e.g., cleavage by a protease enriched or specific in a disease microenvironment) (See Desnoyers et al., Sci Transl Med 5: 207ra144, 2013), chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgAQ1 and IgA2), based on the identity of their heavy-chain constant domains referred to as alpha, delta, epsilon, gamma, and mu, respectively. The different classes of immunoglobulins have different and well known subunit structures and three-dimensional configurations. A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991. National Institutes of Health, Bethesda Md.)); and (2) an approach based on crystallographic studies of antigen-antibody complexes (Al-lazikani et al (1997) J. Molec. Biol. 273:927-948)). In addition, combinations of these two approaches are sometimes used in the art to determine CDRs.

The term “antibody drug conjugate” or “ADC” as used herein refers to a compound that is linked to a cell binding agent (i.e., an antibody or fragment thereof). Typically, the cell binding agent (e.g., antibody) is covalently bound to the drug by a linker.

The term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “cycloalkylene” or “cycloalkylenyl” as used herein refers to a bivalent saturated cycloalkyl radical

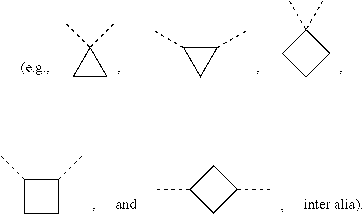

In certain embodiments, the term may be regarded as a product of removal of two hydrogen atoms from the corresponding cycloalkane (e.g., cyclobutyl) by removal of two hydrogen atoms from the same

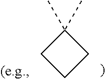

different

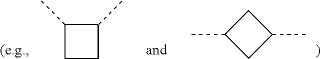

carbon atoms.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “ameliorated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl(2-thienyl, 3-thienyl), furyl(2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl(2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl(2-oxazolyl, 4-oxazolyl, 5-oxazolyl), thiazolyl(2-thiazolyl, 4-thiazolyl, 5-thiazolyl), pyridyl(2-pyridyl, 3-pyridyl, 4-pyridyl), pyrimidinyl(2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl(2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl(2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl(2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl(2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl, C₂-C₃ heterocycloalkyl, C₂-C₄ heterocycloalkyl, C₂-C₅ heterocycloalkyl, C₂-C₆ heterocycloalkyl, C₂-C₇ heterocycloalkyl, C₂-C₈ heterocycloalkyl, C₂-C₉ heterocycloalkyl, C₂-C₁₀ heterocycloalkyl, C₂-C₁₁ heterocycloalkyl, and the like, up to and including a C₂-14₅ heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocycloalkylene” or “heterocycloalkylenyl” as used herein refers to a bivalent saturated cycloalkyl radical

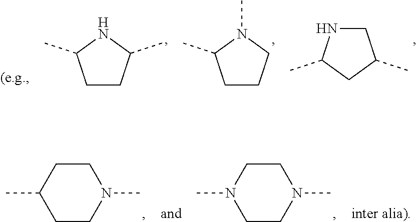

In certain embodiments, the term may be regarded as a product of removal of two hydrogen atoms from the corresponding heterocycloalkane (e.g., piperidine) by removal of two hydrogen atoms from the same

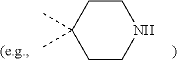

different

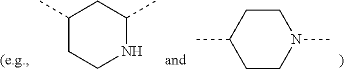

carbon atom(s) and/or heteroatom(s).

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b}) hydrocarbyl, wherein a and b are integers and mean having any of a to b number of carbon atoms. For example, (C₁-C₄) hydrocarbyl means the hydrocarbyl group can be methyl (C₁), ethyl (C₂), propyl (C₃), or butyl (C₄), and (C₀-C_{b}) hydrocarbyl means in certain embodiments there is no hydrocarbyl group. The term “independently selected from” as used herein refers to referenced groups being the same, different, or a mixture thereof, unless the context clearly indicates otherwise.

Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “linker” as used herein refers to an organic moiety that connects two parts of a compound (e.g., a small molecule drug and an antibody). The linker can be, in non-limiting examples, a direct bond, a single atom (e.g., —O—), a peptide, or a substituted or unsubstituted alkylene or heteroalkylene moiety (e.g., polyethylene glycol). One skilled in the art would be apprised of the common linkers suitable for use in antibody drug conjugates and methods of preparation thereof.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo (carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R) CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀) hydrocarbyl, wherein R can be hydrogen (in examples that include other carbon atoms) or a carbon-based moiety, and wherein the carbon-based moiety can be substituted or unsubstituted.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The term “phenylene” or “phenylenyl” as used herein refers to a bivalent phenyl radical (e.g., 1,4-phenylene). In certain embodiments, the term may be regarded as a divalent radical formed by the removal of two hydrogen atoms from a benzene moiety.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

The term “specifically binds”, or “specifically binds”, or the like, means that an antibody or antigen-binding fragment forms a complex with an antigen that is relatively stable under physiological conditions. The specific bond can be characterized by an equilibrium dissociation constant of at least about 5×10⁻⁸ M or less (for example, a smaller K_{D} denotes a firmer bond). Methods for determining whether two molecules specifically bind to each other are well known in the art and include, for example, equilibrium dialysis, surface plasmon resonance, and the like. As described in this document, antibodies have been identified by surface plasmon resonance, for example, BIACORE™, which specifically binds to CD8.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R) CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The terms “treat,” “treating” and “treatment,” as used herein, means reducing the frequency or severity with which symptoms of a disease or condition are experienced by a subject by virtue of administering an agent or compound to the subject.

### Compounds and Compositions

In one aspect, the present disclosure provides a compound of Formula (I), or a salt, solvate, stereoisomer, or isotopologue thereof:

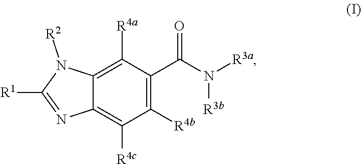

wherein:
R¹ is selected from the group consisting of optionally substituted C₆-C₁₀ aryl and optionally substituted C₂-C₁₂ heteroaryl;
R² is selected from the group consisting of H and optionally substituted C₃-C₈ cycloalkyl;
R^{3a} is selected from the group consisting of optionally substituted C₁-C₆ alkyl, optionally substituted C₇-C₁₃ aralkyl, and

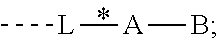

R^{3b} is H,
   or R^{3a} and R^{3b} can combine with the nitrogen to which they are bound to form an optionally substituted C₂-C₁₂ heterocycloalkyl;
R^{4a}, R^{4b}, and R^{4c} are each independently selected from the group consisting of H, halogen, CN, NO₂, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl;
L is selected from the group consisting of (optionally substituted C₁-C₆ alkylenyl)-*,-(optionally substituted C₃-C₆ cycloalkylenyl)-*, -(optionally substituted C₁-C₆ alkylenyl)C(═O)—*, -(optionally substituted C₃-C₆ cycloalkylenyl)C(═O)—*, —C(═O)(optionally substituted C₁-C₆ alkylenyl)-*, and —C(═O)(optionally substituted C₃-C₆ cycloalkylenyl)-*;
A is optionally substituted C₂-C₁₂ heterocyloalkylenyl;
B is selected from the group consisting of optionally substituted C₁-C₆ alkyl, optionally substituted C₃-C₁₂ cycloalkyl, optionally substituted C₇-C₁₃ aralkyl, optionally substituted C₆-C₁₀ aryl, optionally substituted C₂-C₁₂ heteroaryl, C(═O)(optionally substituted C₁-C₆ alkyl), C(═O)(optionally substituted C₃-C₁₂ cycloalkyl), C(═O)(optionally substituted C₇-C₁₃ aralkyl), C(═O)(optionally substituted C₆-C₁₀ aryl), and C(═O)(optionally substituted C₂-C₁₂ heteroaryl); and
* indicates a bond between L and A.

In certain embodiments, R¹ is selected from the group consisting of:

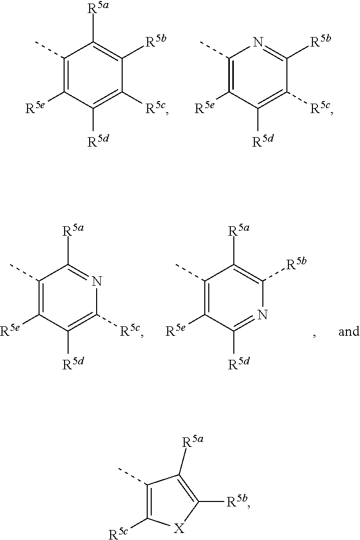

wherein:
R^{5a}, R^{5b}, R^{5c}, R^{5d}, and R^{5e}, if present, are each independently selected from the group consisting of H, C(═O)R^{a}, C(═O)OR^{a}, C(═O)N(R^{a})(R^{b}), N(R^{a})(R^{b}), OR^{a}, halogen, optionally substituted C₁-C₆ alkyl, optionally substituted C₁-C₆ haloalkyl, optionally substituted C₆-C₁₀ aryl, optionally substituted C₂-C₁₂ heteroaryl,
   wherein any two vicinal substituents selected from the group consisting of R^{5a}, R^{5b}, R^{5c}, R^{5d}, and R^{5e} can combine with the atoms to which they are bound to form an optionally substituted C₆-C₁₀ aryl or optionally substituted C₂-C₁₂ heteroaryl;
R⁶ is selected from the group consisting of H, C(═O)OR^{a}, C(═O)N(R^{a})(R^{b}), optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl;
X, if present, is selected from the group consisting of N(R⁶), O, and S; and
R^{a} and R^{b} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.

In certain embodiments, R^{5a} is H. In certain embodiments, R^{5a} is OMe. In certain embodiments, R^{5a} is C(═O)NH₂. In certain embodiments R^{5a} is F. In certain embodiments, R^{5a} is I. In certain embodiments, R^{5a} is phenyl. In certain embodiments, R^{5a} is 4-methoxyphenyl. In certain embodiments, R^{5b} is H. In certain embodiments, R^{5b} is OMe. In certain embodiments, R^{5b} is C(═O)NH₂. In certain embodiments R^{5b} is F. In certain embodiments, R^{5b} is I. In certain embodiments, R^{5b} is phenyl. In certain embodiments, R^{5b} is 4-methoxyphenyl. In certain embodiments, R^{5c} is H. In certain embodiments, R^{5c} is OMe. In certain embodiments, R^{5c} is C(═O)NH₂. In certain embodiments R^{5c} is F. In certain embodiments, R^{5c} is I. In certain embodiments, R^{5c} is phenyl. In certain embodiments, R^{5c} is 4-methoxyphenyl. In certain embodiments, R^{5d} is H. In certain embodiments, R^{5d} is OMe. In certain embodiments, R^{5d} is C(═O)NH₂. In certain embodiments R^{5d} is F. In certain embodiments, R^{5d} is I. In certain embodiments, R^{5d} is phenyl. In certain embodiments, R^{5d} is 4-methoxyphenyl. In certain embodiments, R^{5e} is H. In certain embodiments, R^{5e} is OMe. In certain embodiments, R^{5e} is C(═O)NH₂. In certain embodiments R^{5e} is F. In certain embodiments, R^{5e} is I. In certain embodiments, R^{5e} is phenyl. In certain embodiments, R^{5e} is 4-methoxyphenyl.

In certain embodiments, R¹ is

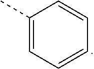

In certain embodiments, R¹ is

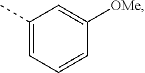

In certain embodiments, R¹ is

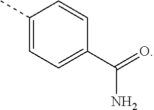

In certain embodiments, R¹ is

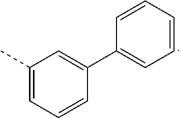

In certain embodiments, R¹ is

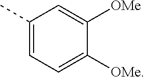

In certain embodiments, R¹ is

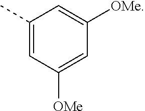

In certain embodiments, R¹ is

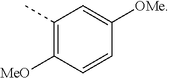

In certain embodiments, R¹ is

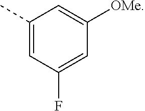

In certain embodiments, R¹ is

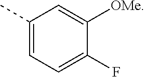

In certain embodiments, R¹ is

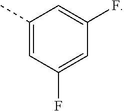

In certain embodiments, R¹ is

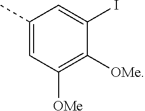

In certain embodiments, R¹ is

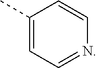

In certain embodiments, R¹ is

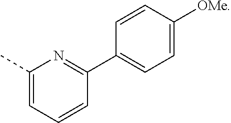

In certain embodiments, R¹ is

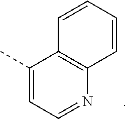

In certain embodiments, R¹ is

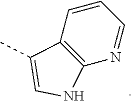

In certain embodiments, R² is selected from the group consisting of:

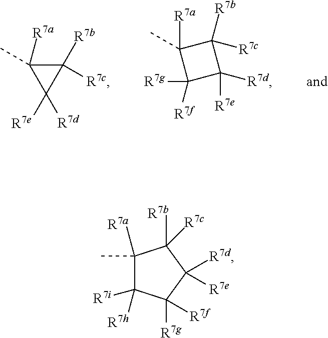

wherein:
R^{7a}, R^{7b}, R^{7c}, R^{7d}, R^{7e}, R^{7f}, R^{7g}, R^{7h}, and R⁷ⁱ, if present, are each independently selected from the group consisting of H, halogen, C(═O)R^{c}, C(═O)OR^{c}, C(═O)N(R^{c})(R^{d}), N(R^{c})(R^{d}), OR^{c}, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl; and
R^{c} and R^{d} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.

In certain embodiments, at least one selected from R^{7a}, R^{7b}, R^{7c}, R^{7d}, R^{7e}, R^{7f}, R^{7g}, R^{7h}, and R⁷ⁱ is H. In certain embodiments, at least two selected from R^{7a}, R^{7b}, R^{7c}, R^{7d}, R^{7e}, R^{7f}, R^{7g}, R^{7h}, and R⁷ⁱ are H. In certain embodiments, at least three selected from R^{7a}, R^{7b}, R^{7c}, R^{7d}, R^{7e}, R^{7f}, R^{7g}, R^{7h}, and R⁷ⁱ are H. In certain embodiments, at least four selected from R^{7a}, R^{7b}, R^{7c}, R^{7d}, R^{7e}, R^{7f}, R^{7g}, R^{7h}, and R⁷ⁱ are H. In certain embodiments, at least five selected from R^{7a}, R^{7b}, R^{7c}, R^{7d}, R^{7e}, R^{7f}, R^{7g}, R^{7h}, and R⁷ⁱ are H. In certain embodiments, at least six selected from R^{7a}, R^{7b}, R^{7c}, R^{7d}, R^{7e}, R^{7f}, R^{7g}, R^{7h}, and R⁷ⁱ are H. In certain embodiments, at least seven selected from R^{7a}, R^{7b}, R^{7c}, R^{7d}, R^{7e}, R^{7f}, R^{7g}, R^{7h}, and R⁷ⁱ are H. In certain embodiments, at least eight selected from R^{7a}, R^{7b}, R^{7c}, R^{7d}, R^{7e}, R^{7f}. R^{7g}, R^{7h}, and R⁷ⁱ are H. In certain embodiments, each of R^{7a}, R^{7b}, R^{7c}, R^{7d}, R^{7e}, R^{7f}, R^{7g}, R^{7h}, and R⁷ⁱ are H.

In certain embodiments, R² is

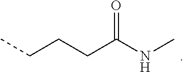

In certain embodiments, R² is

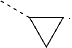

In certain embodiments, R² is

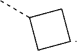

In certain embodiments, R² is

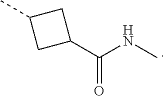

In certain embodiments, R² is

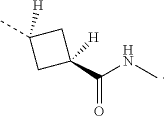

In certain embodiments, R² is

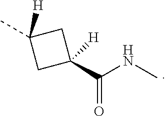

In certain embodiments, R² is

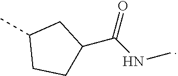

In certain embodiments, R² is

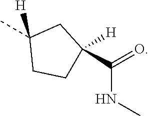

In certain embodiments, R² is

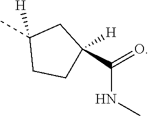

In certain embodiments, R^{3a} is

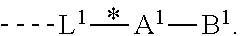

In certain embodiments, L is —CH₂—*. In certain embodiments, L is —CH₂CH₂—*. In certain embodiments, L is —CH₂CH₂CH₂—*. In certain embodiments, L is —CH₂CH₂C(═O)—*. In certain embodiments, L is

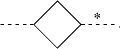

In certain embodiments, A is:

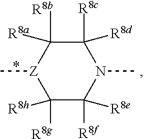

wherein:
R^{8a}, R^{8b}, R^{8c}, R^{8d}, R^{8e}, R^{8f}, R^{8g}, R^{8h}, and R⁸ⁱ, if present, are each independently selected from the group consisting of H, halogen, C(═O)R^{e}, C(═O)OR^{e}, C(═O)N(R^{e})(R^{f}), N(R^{e})(R^{f}), OR^{e}, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl,
   wherein two geminal substituents selected from the group consisting of R^{8a}, R^{8b}, R^{8c}, R^{8d}, R^{8e}, R^{8f}, R^{8g}, and R^{8h} can combine with the carbon atom to which they are bound to form a C(═O);
Z is selected from the group consisting of N and CR⁸ⁱ, and
R^{e} and R^{f} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl

In certain embodiments, at least one selected from R^{8a}, R^{8b}, R^{8c}, R^{8d}, R^{8e}, R^{8f}, R^{8g}, R^{8h}, and R⁸ⁱ is H. In certain embodiments, at least two selected from R^{8a}, R^{8b}, R^{8c}, R^{8d}, R^{8e}, R^{8f}, R^{8g}, R^{8h}, and R⁸ⁱ are H. In certain embodiments, at least three selected from R^{8a}, R^{8b}, R^{8c}, R^{8d}, R^{8e}, R^{8f}, R^{8g}, R^{8h}, and R⁸ⁱ are H. In certain embodiments, at least four selected from R^{8a}, R^{8b}, R^{8c}, R^{8d}, R^{8e}, R^{8f}, R^{8g}, R^{8h}, and R⁸ⁱ are H. In certain embodiments, at least five selected from R^{8a}, R^{8b}, R^{8c}, R^{8d}, R^{8e}, R^{8f}, R^{8g}, R^{8h}, and R⁸ⁱ are H. In certain embodiments, at least six selected from R^{8a}, R^{8b}, R^{8c}, R^{8d}, R^{8e}, R^{8f}, R^{8g}, R^{8h}, and R⁸ⁱ are H. In certain embodiments, at least seven selected from R^{8a}, R^{8b}, R^{8c}, R^{8d}, R^{8e}, R^{8f}, R^{8g}, R^{8h}, and R⁸ⁱ are H. In certain embodiments, at least eight selected from R^{8a}, R^{8b}, R^{8c}, R^{8d}, R^{8e}, R^{8f}, R^{8g}, R^{8h}, and R⁸ⁱ are H. In certain embodiments, each of R^{8a}, R^{8b}, R^{8c}, R^{8d}, R^{8e}, R^{8f}, R^{8g}, R^{8h}, and R⁸ⁱ are H.

In certain embodiments, R^{8a} is H. In certain embodiments, R^{8a} is OH. In certain embodiments R^{8a} is phenyl. In certain embodiments, R^{8b} is H. In certain embodiments, R^{8b} is OH. In certain embodiments R^{8b} is phenyl. In certain embodiments, R^{8c} is H. In certain embodiments, R^{8c} is OH. In certain embodiments R^{8c} is phenyl. In certain embodiments, R^{8d} is H. In certain embodiments, R^{8d} is OH. In certain embodiments R^{8d} is phenyl. In certain embodiments, R^{8e} is H. In certain embodiments, R^{8e} is OH. In certain embodiments R^{8e} is phenyl. In certain embodiments, R^{8f} is H. In certain embodiments, R^{8f} is OH. In certain embodiments R^{8f} is phenyl. In certain embodiments, R^{8g} is H. In certain embodiments, R^{8g} is OH. In certain embodiments R^{8g} is phenyl. In certain embodiments, R^{8h} is H. In certain embodiments, R^{8h} is OH. In certain embodiments R^{8h} is phenyl. In certain embodiments, R⁸ⁱ is H. In certain embodiments, R⁸ⁱ is OH. In certain embodiments R⁸ⁱ is phenyl.

In certain embodiments, R^{8a} and R^{8b} combine with the carbon atoms to which they are bound to form a C(═O). In certain embodiments, R^{8c} and R^{8d} combine with the carbon atoms to which they are bound to form a C(═O). In certain embodiments, R^{8e} and R^{8f} combine with the carbon atoms to which they are bound to form a C(═O). In certain embodiments, R^{8g} and R^{8h} combine with the carbon atoms to which they are bound to form a C(═O).

In certain embodiments, A is

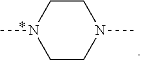

In certain embodiments, A is

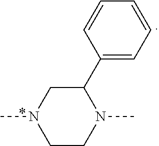

In certain embodiments, A is

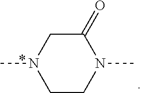

In certain embodiments, A is

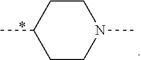

In certain embodiments, A is

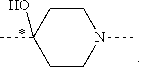

In certain embodiments, B is methyl. In certain embodiments, B is

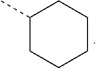

In certain embodiments, B is

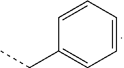

In certain embodiments, B is

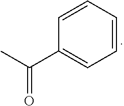

In certain embodiments, B is

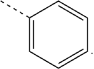

In certain embodiments, B is

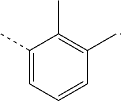

In certain embodiments, B is

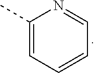

In certain embodiments, R^{3a} and R^{3b} combine to form

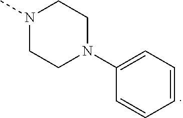

In certain embodiments, R^{3a} is methyl. In certain embodiments, R^{3a} is

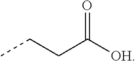

In certain embodiments, R^{3a} is

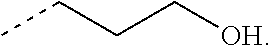

In certain embodiments, R^{3a} is

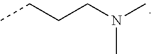

In certain embodiments, R^{3a} is

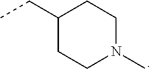

In certain embodiments, R^{3a} is

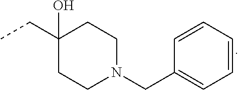

In certain embodiments, R^{3a} is

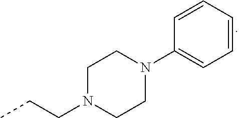

In certain embodiments, R^{3a} is

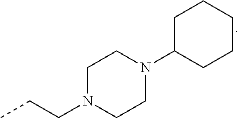

In certain embodiments, R^{3a} is

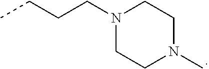

In certain embodiments, R^{3a} is

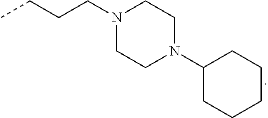

In certain embodiments, R^{3a} is

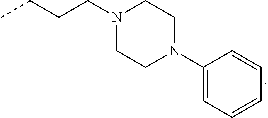

In certain embodiments, R^{3a} is

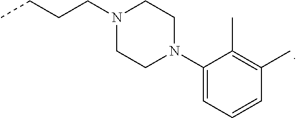

In certain embodiments, R^{3a} is

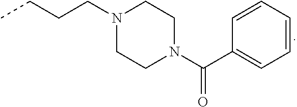

In certain embodiments, R^{3a} is

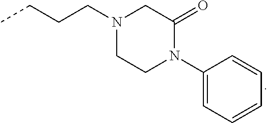

In certain embodiments, R^{3a} is

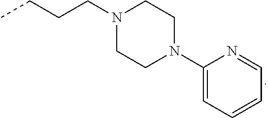

In certain embodiments, R^{3a} is

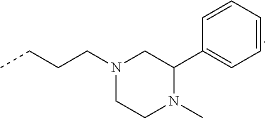

In certain embodiments, R^{3a} is

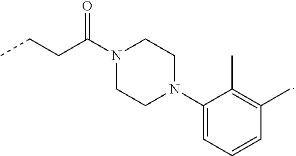

In certain embodiments, R^{3a} is

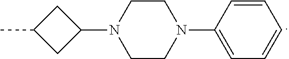

In certain embodiments, R^{3a} is

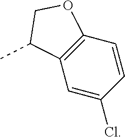

In certain embodiments, each occurrence of alkyl, cycloalkyl, alkylenyl, cycloalkylenyl, heterocycloalkylenyl, heterocycloalkyl, heteroalkyl, aralkyl, aryl, heteroaryl, is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂OR^{I}, N(R^{I})(R^{II}), C₁-C₆ haloalkoxy, C₃-C₈ halocycloalkoxy, aryl, heteroaryl, (C₁-C₆ alkylenyl)C(═O)N(R^{I})(R^{II}), (C₁-C₆ alkylenyl)C(═O)OR^{I}, O(C₁-C₃ alkylenyl)C(═O)OR^{II}, O(C₁-C₃ alkylenyl)C(═O)N(R^{I})(R^{II}), C(═O)R^{I}, C(═O)OR^{I}, OC(═O)R^{I}, OC(═O)OR^{I}, SR^{I}, S(═O)R^{I}, S(═O)₂R^{I}, S(═O)₂N(R^{I})(R^{II}), S(═O)₂NR^{I}C(═O)NHR^{II}, N(R^{I})S(═O)₂R^{II}, N(R^{I})C(═O)R^{II}, and C(═O)NR^{I}R^{II}, wherein R^{I} and R^{II} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

In certain embodiments, the compound is selected from the group consisting of:
2-(3-iodo-4,5-dimethoxyphenyl)-1-((1S,3S)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-((1R,3S)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-((1S,3R)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-((1R,3R)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-(3-(methylcarbamoyl)cyclopentyl)-N-(3-(4-phenylpiperazin-1-yl)propyl)-1H-benzo[d]imidazole-6-carboxamide;
1-((1S,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1R,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1S,3R)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1R,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-(3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
N-(5-chloro-2,3-dihydrobenzofuran-3-yl)-1-((1S,3S)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-((1R,3S)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-((1S,3R)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-((1R,3R)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-(3-(methylcarbamoyl)cyclopentyl)-2-(3,4,5-trimethoxyphenyl)-1H-benzo[d]imidazole-6-carboxamide;
N-methyl-1-((1S,3S)-3-(methylcarbamoyl)cyclopentyl)-2-(3,4,5-trimethoxyphenyl)-1H-benzo[d]imidazole-6-carboxamide;
N-methyl-1-((1R,3S)-3-(methylcarbamoyl)cyclopentyl)-2-(3,4,5-trimethoxyphenyl)-1H-benzo[d]imidazole-6-carboxamide;
N-methyl-1-((1S,3R)-3-(methylcarbamoyl)cyclopentyl)-2-(3,4,5-trimethoxyphenyl)-1H-benzo[d]imidazole-6-carboxamide;
N-methyl-1-((1R,3R)-3-(methylcarbamoyl)cyclopentyl)-2-(3,4,5-trimethoxyphenyl)-1H-benzo[d]imidazole-6-carboxamide;
N-methyl-1-(3-(methylcarbamoyl)cyclopentyl)-2-(3,4,5-trimethoxyphenyl)-1H-benzo[d]imidazole-6-carboxamide;
1-((1r,3r)-3-(methylcarbamoyl)cyclobutyl)-N-(3-(4-phenylpiperazin-1-yl)propyl)-2-(3,4,5-trimethoxyphenyl)-1H-benzo[d]imidazole-6-carboxamide;
1-((1r,3s)-3-(methylcarbamoyl)cyclobutyl)-N-(3-(4-phenylpiperazin-1-yl)propyl)-2-(3,4,5-trimethoxyphenyl)-1H-benzo[d]imidazole-6-carboxamide;
1-(3-(methylcarbamoyl)cyclobutyl)-N-(3-(4-phenylpiperazin-1-yl)propyl)-2-(3,4,5-trimethoxyphenyl)-1H-benzo[d]imidazole-6-carboxamide;
N-(3-(4-phenylpiperazin-1-yl)propyl)-2-(3,4,5-trimethoxyphenyl)-1H-benzo[d]imidazole-6-carboxamide;
(1r,3r)-N-methyl-3-(6-(4-phenylpiperazine-1-carbonyl)-2-(3,4,5-trimethoxyphenyl)-1H-benzo[d]imidazol-1-yl)cyclobutane-1-carboxamide;
(1r,3s)-N-methyl-3-(6-(4-phenylpiperazine-1-carbonyl)-2-(3,4,5-trimethoxyphenyl)-1H-benzo[d]imidazol-1-yl)cyclobutane-1-carboxamide;
N-methyl-3-(6-(4-phenylpiperazine-1-carbonyl)-2-(3,4,5-trimethoxyphenyl)-1H-benzo[d]imidazol-1-yl)cyclobutane-1-carboxamide;
1-((1r,3r)-3-(methylcarbamoyl)cyclobutyl)-N-(3-(4-methylpiperazin-1-yl)propyl)-2-(3,4,5-trimethoxyphenyl)-1H-benzo[d]imidazole-6-carboxamide;
1-((1r,3s)-3-(methylcarbamoyl)cyclobutyl)-N-(3-(4-methylpiperazin-1-yl)propyl)-2-(3,4,5-trimethoxyphenyl)-1H-benzo[d]imidazole-6-carboxamide;
1-(3-(methylcarbamoyl)cyclobutyl)-N-(3-(4-methylpiperazin-1-yl)propyl)-2-(3,4,5-trimethoxyphenyl)-1H-benzo[d]imidazole-6-carboxamide;
N-(3-(dimethylamino)propyl)-1-((1r,3r)-3-(methylcarbamoyl)cyclobutyl)-2-(3,4,5-trimethoxyphenyl)-1H-benzo[d]imidazole-6-carboxamide;
N-(3-(dimethylamino)propyl)-1-((1r,3s)-3-(methylcarbamoyl)cyclobutyl)-2-(3,4,5-trimethoxyphenyl)-1H-benzo[d]imidazole-6-carboxamide;
N-(3-(dimethylamino)propyl)-1-(3-(methylcarbamoyl)cyclobutyl)-2-(3,4,5-trimethoxyphenyl)-1H-benzo[d]imidazole-6-carboxamide;
1-((1r,3r)-3-(methylcarbamoyl)cyclobutyl)-N-((1-methylpiperidin-4-yl)methyl)-2-(3,4,5-trimethoxyphenyl)-1H-benzo[d]imidazole-6-carboxamide;
1-((1r,3s)-3-(methylcarbamoyl)cyclobutyl)-N-((1-methylpiperidin-4-yl)methyl)-2-(3,4,5-trimethoxyphenyl)-1H-benzo[d]imidazole-6-carboxamide;
1-(3-(methylcarbamoyl)cyclobutyl)-N-((1-methylpiperidin-4-yl)methyl)-2-(3,4,5-trimethoxyphenyl)-1H-benzo[d]imidazole-6-carboxamide;
2-(3-methoxyphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(3-methoxyphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(3-methoxyphenyl)-1-(3-(methylcarbamoyl)cyclobutyl)-N-(3-(4-phenylpiperazin-1-yl)propyl)-1H-benzo[d]imidazole-6-carboxamide;
2-(3,5-dimethoxyphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(3,5-dimethoxyphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(3,5-dimethoxyphenyl)-1-(3-(methylcarbamoyl)cyclobutyl)-N-(3-(4-phenylpiperazin-1-yl)propyl)-1H-benzo[d]imidazole-6-carboxamide;
1-cyclobutyl-N-(3-(4-phenylpiperazin-1-yl)propyl)-2-(3,4,5-trimethoxyphenyl)-1H-benzo[d]imidazole-6-carboxamide;
1-(4-(methylamino)-4-oxobutyl)-N-(3-(4-phenylpiperazin-1-yl)propyl)-2-(3,4,5-trimethoxyphenyl)-1H-benzo[d]imidazole-6-carboxamide;
1-((1r,3r)-3-(methylcarbamoyl)cyclobutyl)-2-phenyl-N-(3-(4-phenylpiperazin-1-yl)propyl)-1H-benzo[d]imidazole-6-carboxamide;
1-((1r,3s)-3-(methylcarbamoyl)cyclobutyl)-2-phenyl-N-(3-(4-phenylpiperazin-1-yl)propyl)-1H-benzo[d]imidazole-6-carboxamide;
1-(3-(methylcarbamoyl)cyclobutyl)-2-phenyl-N-(3-(4-phenylpiperazin-1-yl)propyl)-1H-benzo[d]imidazole-6-carboxamide;
2-(4-carbamoylphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(4-carbamoylphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(4-carbamoylphenyl)-1-(3-(methylcarbamoyl)cyclobutyl)-N-(3-(4-phenylpiperazin-1-yl)propyl)-1H-benzo[d]imidazole-6-carboxamide;
2-(3-fluoro-5-methoxyphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(3-fluoro-5-methoxyphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(3-fluoro-5-methoxyphenyl)-1-(3-(methylcarbamoyl)cyclobutyl)-N-(3-(4-phenylpiperazin-1-yl)propyl)-1H-benzo[d]imidazole-6-carboxamide;
N-(3-(4-(2,3-dimethylphenyl)piperazin-1-yl)propyl)-1-((1r,3r)-3-(methylcarbamoyl)cyclobutyl)-2-(3,4,5-trimethoxyphenyl)-1H-benzo[d]imidazole-6-carboxamide;
N-(3-(4-(2,3-dimethylphenyl)piperazin-1-yl)propyl)-1-((1r,3s)-3-(methylcarbamoyl)cyclobutyl)-2-(3,4,5-trimethoxyphenyl)-1H-benzo[d]imidazole-6-carboxamide;
N-(3-(4-(2,3-dimethylphenyl)piperazin-1-yl)propyl)-1-(3-(methylcarbamoyl)cyclobutyl)-2-(3,4,5-trimethoxyphenyl)-1H-benzo[d]imidazole-6-carboxamide;
1-cyclobutyl-N-(3-(4-(2,3-dimethylphenyl)piperazin-1-yl)propyl)-2-(3,4,5-trimethoxyphenyl)-1H-benzo[d]imidazole-6-carboxamide;
2-(2,5-dimethoxyphenyl)-N-(3-(4-(2,3-dimethylphenyl)piperazin-1-yl)propyl)-1-((1r,3r)-3-(methylcarbamoyl)cyclobutyl)-1H-benzo[d]imidazole-6-carboxamide;
2-(2,5-dimethoxyphenyl)-N-(3-(4-(2,3-dimethylphenyl)piperazin-1-yl)propyl)-1-((1r,3s)-3-(methylcarbamoyl)cyclobutyl)-1H-benzo[d]imidazole-6-carboxamide;
2-(2,5-dimethoxyphenyl)-N-(3-(4-(2,3-dimethylphenyl)piperazin-1-yl)propyl)-1-(3-(methylcarbamoyl)cyclobutyl)-1H-benzo[d]imidazole-6-carboxamide;
2-(3,4-dimethoxyphenyl)-N-(3-(4-(2,3-dimethylphenyl)piperazin-1-yl)propyl)-1-((1r,3r)-3-(methylcarbamoyl)cyclobutyl)-1H-benzo[d]imidazole-6-carboxamide;
2-(3,4-dimethoxyphenyl)-N-(3-(4-(2,3-dimethylphenyl)piperazin-1-yl)propyl)-1-((1r,3s)-3-(methylcarbamoyl)cyclobutyl)-1H-benzo[d]imidazole-6-carboxamide;
2-(3,4-dimethoxyphenyl)-N-(3-(4-(2,3-dimethylphenyl)piperazin-1-yl)propyl)-1-(3-(methylcarbamoyl)cyclobutyl)-1H-benzo[d]imidazole-6-carboxamide;
3-(1-cyclobutyl-2-(3,4,5-trimethoxyphenyl)-1H-benzo[d]imidazole-6-carboxamido)propanoic acid;
1-cyclobutyl-N-(3-hydroxypropyl)-2-(3,4,5-trimethoxyphenyl)-1H-benzo[d]imidazole-6-carboxamide;
1-cyclobutyl-N-(3-(4-(2,3-dimethylphenyl)piperazin-1-yl)-3-oxopropyl)-2-(3,4,5-trimethoxyphenyl)-1H-benzo[d]imidazole-6-carboxamide;
1-cyclopropyl-N-(3-(4-(2,3-dimethylphenyl)piperazin-1-yl)propyl)-2-(3,4,5-trimethoxyphenyl)-1H-benzo[d]imidazole-6-carboxamide;
1-cyclobutyl-2-(3,5-dimethoxyphenyl)-N-(3-(4-(2,3-dimethylphenyl)piperazin-1-yl)propyl)-1H-benzo[d]imidazole-6-carboxamide;
1-cyclobutyl-N-(3-(dimethylamino)propyl)-2-(3-fluoro-5-methoxyphenyl)-1H-benzo[d]imidazole-6-carboxamide;
1-cyclobutyl-2-(3,5-difluorophenyl)-N-(3-(dimethylamino)propyl)-1H-benzo[d]imidazole-6-carboxamide;
1-cyclobutyl-N-(3-(dimethylamino)propyl)-2-(pyridin-4-yl)-1H-benzo[d]imidazole-6-carboxamide;
1-cyclobutyl-N-(3-(dimethylamino)propyl)-2-(6-(4-methoxyphenyl)pyridin-2-yl)-1H-benzo[d]imidazole-6-carboxamide;
2-([1,1′-biphenyl]-3-yl)-1-cyclobutyl-N-(3-(dimethylamino)propyl)-1H-benzo[d]imidazole-6-carboxamide;
1-((1R,3R)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1S,3R)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1R,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1S,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-(3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-cyclobutyl-N-(3-(dimethylamino)propyl)-2-(isoquinolin-4-yl)-1H-benzo[d]imidazole-6-carboxamide;
1-cyclobutyl-N-(3-(dimethylamino)propyl)-2-(quinolin-4-yl)-1H-benzo[d]imidazole-6-carboxamide;
1-cyclobutyl-N-(3-(dimethylamino)propyl)-2-(1H-pyrrolo[2,3-b]pyridin-3-yl)-1H-benzo[d]imidazole-6-carboxamide;
N-(3-(dimethylamino)propyl)-2-(3-fluoro-5-methoxyphenyl)-1-((1r,3r)-3-(methylcarbamoyl)cyclobutyl)-1H-benzo[d]imidazole-6-carboxamide;
N-(3-(dimethylamino)propyl)-2-(3-fluoro-5-methoxyphenyl)-1-((1r,3s)-3-(methylcarbamoyl)cyclobutyl)-1H-benzo[d]imidazole-6-carboxamide;
N-(3-(dimethylamino)propyl)-2-(3-fluoro-5-methoxyphenyl)-1-(3-(methylcarbamoyl)cyclobutyl)-1H-benzo[d]imidazole-6-carboxamide;
N-(3-(dimethylamino)propyl)-2-(4-fluoro-3-methoxyphenyl)-1-((1r,3r)-3-(methylcarbamoyl)cyclobutyl)-1H-benzo[d]imidazole-6-carboxamide;
N-(3-(dimethylamino)propyl)-2-(4-fluoro-3-methoxyphenyl)-1-((1r,3s)-3-(methylcarbamoyl)cyclobutyl)-1H-benzo[d]imidazole-6-carboxamide;
N-(3-(dimethylamino)propyl)-2-(4-fluoro-3-methoxyphenyl)-1-(3-(methylcarbamoyl)cyclobutyl)-1H-benzo[d]imidazole-6-carboxamide;
N-(3-(dimethylamino)propyl)-2-(3-methoxyphenyl)-1-((1r,3r)-3-(methylcarbamoyl)cyclobutyl)-1H-benzo[d]imidazole-6-carboxamide;
N-(3-(dimethylamino)propyl)-2-(3-methoxyphenyl)-1-((1r,3s)-3-(methylcarbamoyl)cyclobutyl)-1H-benzo[d]imidazole-6-carboxamide;
N-(3-(dimethylamino)propyl)-2-(3-methoxyphenyl)-1-(3-(methylcarbamoyl)cyclobutyl)-1H-benzo[d]imidazole-6-carboxamide;
2-(3,4-dimethoxyphenyl)-N-(3-(dimethylamino)propyl)-1-((1r,3r)-3-(methylcarbamoyl)cyclobutyl)-1H-benzo[d]imidazole-6-carboxamide;
2-(3,4-dimethoxyphenyl)-N-(3-(dimethylamino)propyl)-1-((1r,3s)-3-(methylcarbamoyl)cyclobutyl)-1H-benzo[d]imidazole-6-carboxamide;
2-(3,4-dimethoxyphenyl)-N-(3-(dimethylamino)propyl)-1-(3-(methylcarbamoyl)cyclobutyl)-1H-benzo[d]imidazole-6-carboxamide;
2-(3,5-dimethoxyphenyl)-N-(3-(dimethylamino)propyl)-1-((1r,3r)-3-(methylcarbamoyl)cyclobutyl)-1H-benzo[d]imidazole-6-carboxamide;
2-(3,5-dimethoxyphenyl)-N-(3-(dimethylamino)propyl)-1-((1r,3s)-3-(methylcarbamoyl)cyclobutyl)-1H-benzo[d]imidazole-6-carboxamide;
2-(3,5-dimethoxyphenyl)-N-(3-(dimethylamino)propyl)-1-(3-(methylcarbamoyl)cyclobutyl)-1H-benzo[d]imidazole-6-carboxamide;
2-(2,5-dimethoxyphenyl)-N-(3-(dimethylamino)propyl)-1-((1r,3r)-3-(methylcarbamoyl)cyclobutyl)-1H-benzo[d]imidazole-6-carboxamide;
2-(2,5-dimethoxyphenyl)-N-(3-(dimethylamino)propyl)-1-((1r,3s)-3-(methylcarbamoyl)cyclobutyl)-1H-benzo[d]imidazole-6-carboxamide;
2-(2,5-dimethoxyphenyl)-N-(3-(dimethylamino)propyl)-1-(3-(methylcarbamoyl)cyclobutyl)-1H-benzo[d]imidazole-6-carboxamide;
1-cyclobutyl-N-(2-(4-phenylpiperazin-1-yl)ethyl)-2-(3,4,5-trimethoxyphenyl)-1H-benzo[d]imidazole-6-carboxamide;
1-cyclobutyl-N-(3-(4-(pyridin-2-yl)piperazin-1-yl)propyl)-2-(3,4,5-trimethoxyphenyl)-1H-benzo[d]imidazole-6-carboxamide;
1-cyclobutyl-N-(3-(4-phenylpiperazin-1-yl)cyclobutyl)-2-(3,4,5-trimethoxyphenyl)-1H-benzo[d]imidazole-6-carboxamide;
1-cyclobutyl-N-(3-(dimethylamino)propyl)-2-(3,4,5-trimethoxyphenyl)-1H-benzo[d]imidazole-6-carboxamide;
1-cyclobutyl-N-(3-(3-oxo-4-phenylpiperazin-1-yl)propyl)-2-(3,4,5-trimethoxyphenyl)-1H-benzo[d]imidazole-6-carboxamide;
N-(3-(4-benzoylpiperazin-1-yl)propyl)-1-cyclobutyl-2-(3,4,5-trimethoxyphenyl)-1H-benzo[d]imidazole-6-carboxamide;
1-cyclobutyl-N-(3-(4-cyclohexylpiperazin-1-yl)propyl)-2-(3,4,5-trimethoxyphenyl)-1H-benzo[d]imidazole-6-carboxamide;
1-cyclobutyl-N-(3-(4-methyl-3-phenylpiperazin-1-yl)propyl)-2-(3,4,5-trimethoxyphenyl)-1H-benzo[d]imidazole-6-carboxamide;
1-cyclobutyl-N-(2-(4-cyclohexylpiperazin-1-yl)ethyl)-2-(3,4,5-trimethoxyphenyl)-1H-benzo[d]imidazole-6-carboxamide; and
N-((1-benzyl-4-hydroxypiperidin-4-yl)methyl)-1-cyclobutyl-2-(3,4,5-trimethoxyphenyl)-1H-benzo[d]imidazole-6-carboxamide.

In another aspect, the present disclosure provides a pharmaceutical composition comprising at least one compound of the present disclosure and a pharmaceutically acceptable carrier.

In certain embodiments, the pharmaceutical composition further comprises at least one additional therapeutically effective agent.

The compounds described herein can possess one or more stereocenters, and each stereocenter can exist independently in either the (R) or(S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from allyl, benzyl (Bn), benzyloxycarbonyl (Cbz), allyloxycarbonyl (Alloc), methyl, ethyl, t-butyl, t-butyldimethylsilyl (TBDMS), 2-(trimethylsilyl) ethoxycarbonyl (Teoc), t-butyloxycarbonyl (Boc), para-methoxybenzyl (PMB), triphenylmethyl (trityl), acetyl, and fluorenylmethoxycarbonyl (FMOC).

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

TABLE 1
Exemplary compounds of the present disclosure

| Cmpd | Structure |
|---|---|
| 1 CDD-2205 | 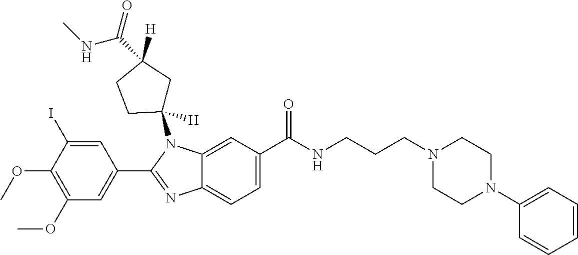 [SMILES: CNC(=O)[C@H]1CC[C@H](n2c(-c3cc(I)c(OC)c(OC)c3)nc3ccc(C(=O)NCCCN4CCN(c5ccccc5)CC4)cc32)C1] |
| | 2-(3-iodo-4,5-dimethoxyphenyl)-1-((1S,3S)-3-(methylcarbamoyl)cyclopentyl)-N- |
| | (3-(4-phenylpiperazin-1-yl)propyl)-1H-benzo[d]imidazole-6-carboxamide |
| 2 CDD-2206 | 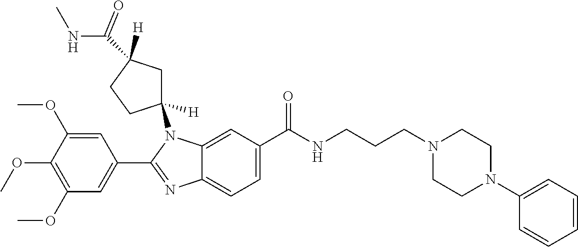 [SMILES: CNC(=O)[C@H]1CC[C@H](n2c(-c3cc(OC)c(OC)c(OC)c3)nc3ccc(C(=O)NCCCN4CCN(c5ccccc5)CC4)cc32)C1] |
| | 1-((1S,3S)-3-(methylcarbamoyl)cyclopentyl)-N-(3-(4-phenylpiperazin-1- |
| | yl)propyl)-2-(3,4,5-trimethoxyphenyl)-1H-benzo[d]imidazole-6-carboxamide |
| 3 CDD-2233 | 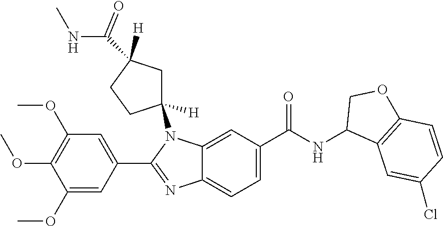 [SMILES: CNC(=O)[C@H]1CC[C@H](n2c(-c3cc(OC)c(OC)c(OC)c3)nc3ccc(C(=O)NC4COc5ccc(Cl)cc54)cc32)C1] |
| | N-(5-chloro-2,3-dihydrobenzofuran-3-yl)-1-((1S,3S)-3- |
| | (methylcarbamoyl)cyclopentyl)-2-(3,4,5-trimethoxyphenyl)-1H- |
| | benzo[d]imidazole-6-carboxamide |
| 4 CDD-2234 | 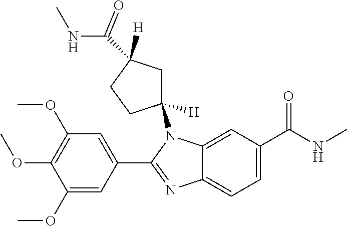 |
| | N-methyl-1-((1S,3S)-3-(methylcarbamoyl)cyclopentyl)-2-(3,4,5- |
| | trimethoxyphenyl)-1H-benzo[d]imidazole-6-carboxamide |
| 5 CDD-2235 | 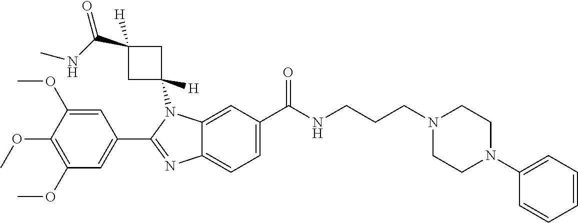 [SMILES: CNC(=O)[C@H]1C[C@H](n2c(-c3cc(OC)c(OC)c(OC)c3)nc3ccc(C(=O)NCCCN4CCN(c5ccccc5)CC4)cc32)C1] |
| | 1-((1r,3r)-3-(methylcarbamoyl)cyclobutyl)-N-(3-(4-phenylpiperazin-1-yl)propyl)- |
| | 2-(3,4,5-trimethoxyphenyl)-1H-benzo[d]imidazole-6-carboxamide |
| 6 CDD-2236 | 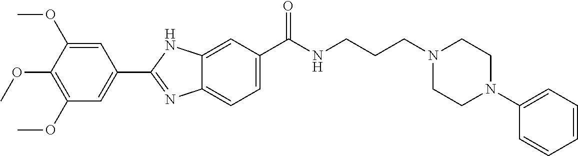 [SMILES: COc1cc(-c2nc3ccc(C(=O)NCCCN4CCN(c5ccccc5)CC4)cc3[nH]2)cc(OC)c1OC] |
| | N-(3-(4-phenylpiperazin-1-yl)propyl)-2-(3,4,5-trimethoxyphenyl)-1H- |
| | benzo[d]imidazole-6-carboxamide |
| 7 CDD-2279 | 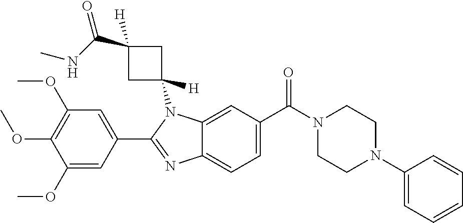 [SMILES: CNC(=O)[C@H]1C[C@H](n2c(-c3cc(OC)c(OC)c(OC)c3)nc3ccc(C(=O)N4CCN(c5ccccc5)CC4)cc32)C1] |
| | (1r,3r)-N-methyl-3-(6-(4-phenylpiperazine-1-carbonyl)-2-(3,4,5- |
| | trimethoxyphenyl)-1H-benzo[d]imidazol-1-yl)cyclobutane-1-carboxamide |
| 8 CDD-2280 | 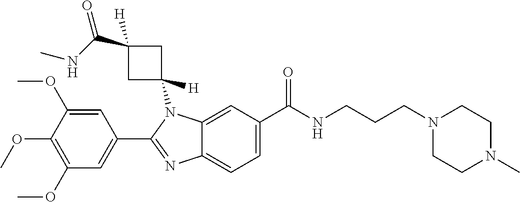 |
| | 1-((1r,3r)-3-(methylcarbamoyl)cyclobutyl)-N-(3-(4-methylpiperazin-1- |
| | yl)propyl)-2-(3,4,5-trimethoxyphenyl)-1H-benzo[d]imidazole-6-carboxamide |
| 9 CDD-2281 | 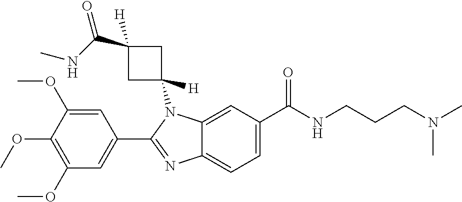 |
| | N-(3-(dimethylamino)propyl)-1-((1r,3r)-3-(methylcarbamoyl)cyclobutyl)-2- |
| | (3,4,5-trimethoxyphenyl)-1H-benzo[d]imidazole-6-carboxamide |
| 10 CDD-2282 | 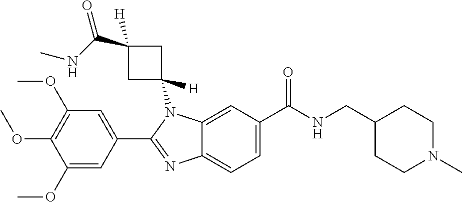 |
| | 1-((1r,3r)-3-(methylcarbamoyl)cyclobutyl)-N-((1-methylpiperidin-4-yl)methyl)- |
| | 2-(3,4,5-trimethoxyphenyl)-1H-benzo[d]imidazole-6-carboxamide |
| 11 CDD-2313 | 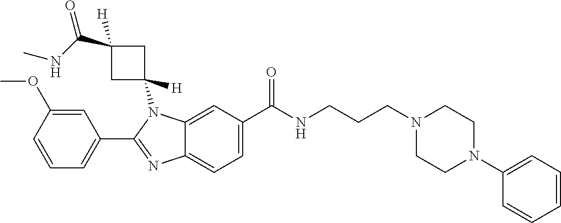 [SMILES: CNC(=O)[C@H]1C[C@H](n2c(-c3cccc(OC)c3)nc3ccc(C(=O)NCCCN4CCN(c5ccccc5)CC4)cc32)C1] |
| | 2-(3-methoxyphenyl)-1-((1r,3r)-3-(methylcarbamoyl)cyclobutyl)-N-(3-(4- |
| | phenylpiperazin-1-yl)propyl)-1H-benzo[d]imidazole-6-carboxamide |
| 12 CDD-2314 | 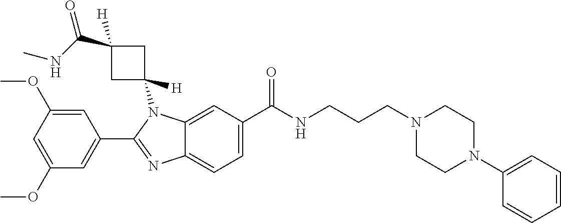 [SMILES: CNC(=O)[C@H]1C[C@H](n2c(-c3cc(OC)cc(OC)c3)nc3ccc(C(=O)NCCCN4CCN(c5ccccc5)CC4)cc32)C1] |
| | 2-(3,5-dimethoxyphenyl)-1-((1r,3r)-3-(methylcarbamoyl)cyclobutyl)-N-(3-(4- |
| | phenylpiperazin-1-yl)propyl)-1H-benzo[d]imidazole-6-carboxamide |
| 13 CDD-2315 | 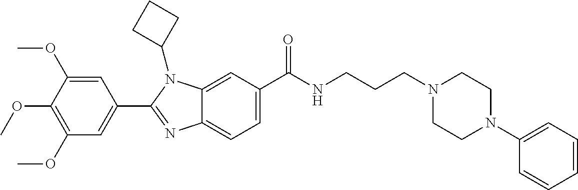 [SMILES: COc1cc(-c2nc3ccc(C(=O)NCCCN4CCN(c5ccccc5)CC4)cc3n2C2CCC2)cc(OC)c1OC] |
| | 1-cyclobutyl-N-(3-(4-phenylpiperazin-1-yl)propyl)-2-(3,4,5-trimethoxyphenyl)- |
| | 1H-benzo[d]imidazole-6-carboxamide |
| 14 CDD-2316 | 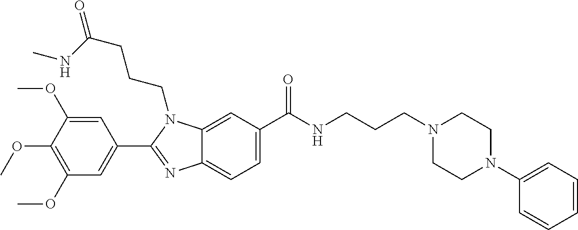 [SMILES: CNC(=O)CCCn1c(-c2cc(OC)c(OC)c(OC)c2)nc2ccc(C(=O)NCCCN3CCN(c4ccccc4)CC3)cc21] |
| | 1-(4-(methylamino)-4-oxobutyl)-N-(3-(4-phenylpiperazin-1-yl)propyl)-2-(3,4,5- |
| | trimethoxyphenyl)-1H-benzo[d]imidazole-6-carboxamide |
| 15 CDD-2317 | 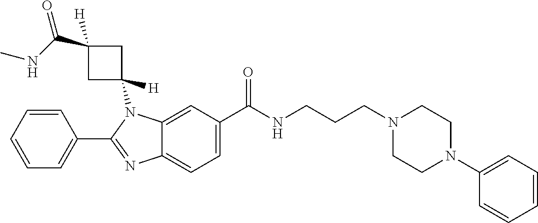 [SMILES: CNC(=O)[C@H]1C[C@H](n2c(-c3ccccc3)nc3ccc(C(=O)NCCCN4CCN(c5ccccc5)CC4)cc32)C1] |
| | 1-((1r,3r)-3-(methylcarbamoyl)cyclobutyl)-2-phenyl-N-(3-(4-phenylpiperazin-1- |
| | yl)propyl)-1H-benzo[d]imidazole-6-carboxamide |
| 16 CDD-2339 | 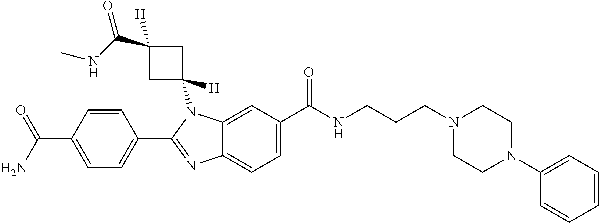 [SMILES: CNC(=O)[C@H]1C[C@H](n2c(-c3ccc(C(N)=O)cc3)nc3ccc(C(=O)NCCCN4CCN(c5ccccc5)CC4)cc32)C1] |
| | 2-(4-carbamoylphenyl)-1-((1r,3r)-3-(methylcarbamoyl)cyclobutyl)-N-(3-(4- |
| | phenylpiperazin-1-yl)propyl)-1H-benzo[d]imidazole-6-carboxamide |
| 17 CDD-2340 | 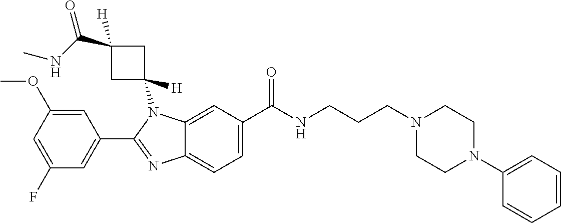 [SMILES: CNC(=O)[C@H]1C[C@H](n2c(-c3cc(F)cc(OC)c3)nc3ccc(C(=O)NCCCN4CCN(c5ccccc5)CC4)cc32)C1] |
| | 2-(3-fluoro-5-methoxyphenyl)-1-((1r,3r)-3-(methylcarbamoyl)cyclobutyl)-N-(3- |
| | (4-phenylpiperazin-1-yl)propyl)-1H-benzo[d]imidazole-6-carboxamide |
| 18 CDD-2341 | 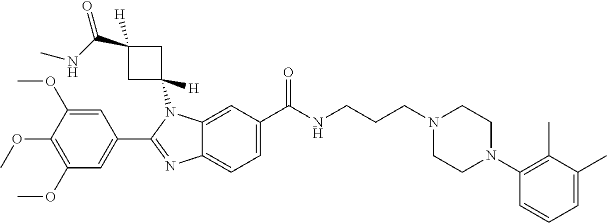 |
| | N-(3-(4-(2,3-dimethylphenyl)piperazin-1-yl)propyl)-1-((1r,3r)-3- |
| | (methylcarbamoyl)cyclobutyl)-2-(3,4,5-trimethoxyphenyl)-1H- |
| | benzo[d]imidazole-6-carboxamide |
| 19 CDD-2342 | 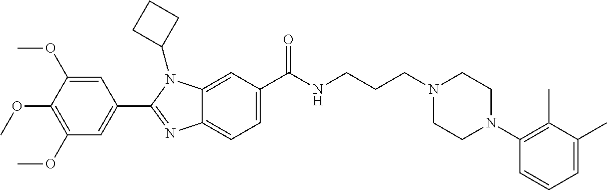 |
| | 1-cyclobutyl-N-(3-(4-(2,3-dimethylphenyl)piperazin-1-yl)propyl)-2-(3,4,5- |
| | trimethoxyphenyl)-1H-benzo[d]imidazole-6-carboxamide |
| 20 CDD-2420 | 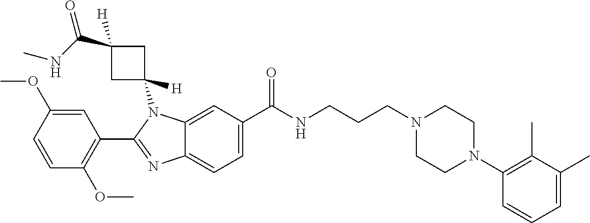 |
| | 2-(2,5-dimethoxyphenyl)-N-(3-(4-(2,3-dimethylphenyl)piperazin-1-yl)propyl)-1- |
| | ((1r,3r)-3-(methylcarbamoyl)cyclobutyl)-1H-benzo[d]imidazole-6-carboxamide |
| 21 CDD-2421 | 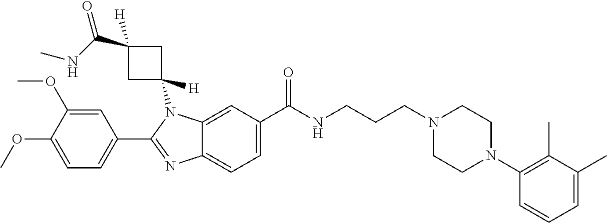 |
| | 2-(3,4-dimethoxyphenyl)-N-(3-(4-(2,3-dimethylphenyl)piperazin-1-yl)propyl)-1- |
| | ((1r,3r)-3-(methylcarbamoyl)cyclobutyl)-1H-benzo[d]imidazole-6-carboxamide |
| 22 CDD-2464 | 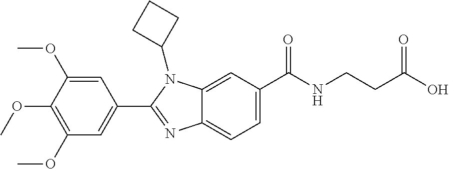 [SMILES: COc1cc(-c2nc3ccc(C(=O)NCCC(=O)O)cc3n2C2CCC2)cc(OC)c1OC] |
| | 3-(1-cyclobutyl-2-(3,4,5-trimethoxyphenyl)-1H-benzo[d]imidazole-6- |
| | carboxamido)propanoic acid |
| 23 CDD-2465 | 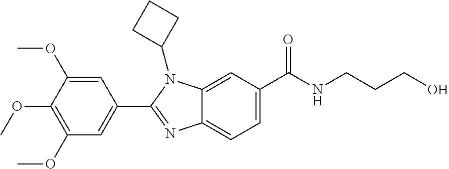 [SMILES: COc1cc(-c2nc3ccc(C(=O)NCCCO)cc3n2C2CCC2)cc(OC)c1OC] |
| | 1-cyclobutyl-N-(3-hydroxypropyl)-2-(3,4,5-trimethoxyphenyl)-1H- |
| | benzo[d]imidazole-6-carboxamide |
| 24 CDD-2467 | 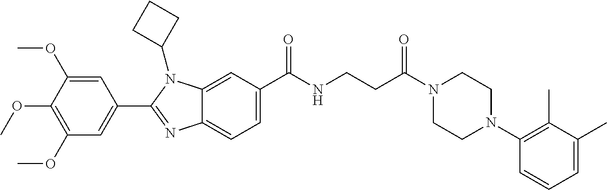 |
| | 1-cyclobutyl-N-(3-(4-(2,3-dimethylphenyl)piperazin-1-yl)-3-oxopropyl)-2-(3,4,5- |
| | trimethoxyphenyl)-1H-benzo[d]imidazole-6-carboxamide |
| 25 CDD-2468 | 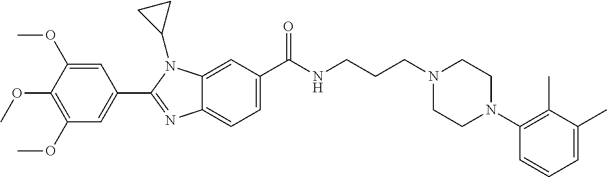 |
| | 1-cyclopropyl-N-(3-(4-(2,3-dimethylphenyl)piperazin-1-yl)propyl)-2-(3,4,5- |
| | trimethoxyphenyl)-1H-benzo[d]imidazole-6-carboxamide |
| 26 CDD-2469 | 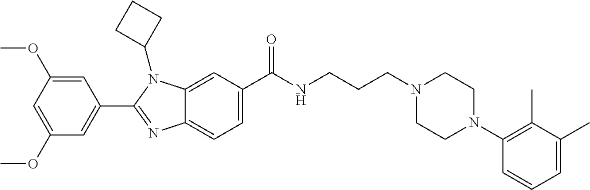 |
| | 1-cyclobutyl-2-(3,5-dimethoxyphenyl)-N-(3-(4-(2,3-dimethylphenyl)piperazin-1- |
| | yl)propyl)-1H-benzo[d]imidazole-6-carboxamide |
| 27 CDD-2617 | 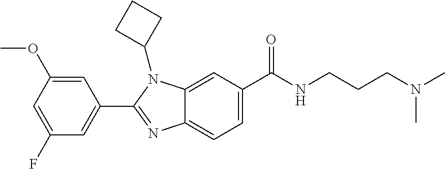 |
| | 1-cyclobutyl-N-(3-(dimethylamino)propyl)-2-(3-fluoro-5-methoxyphenyl)-1H- |
| | benzo[d]imidazole-6-carboxamide |
| 28 CDD-2618 | 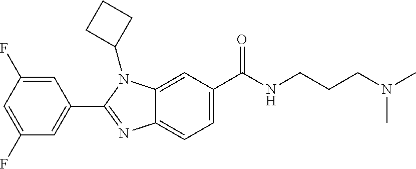 [SMILES: CN(C)CCCNC(=O)c1ccc2nc(-c3cc(F)cc(F)c3)n(C3CCC3)c2c1] |
| | 1-cyclobutyl-2-(3,5-difluorophenyl)-N-(3-(dimethylamino)propyl)-1H- |
| | benzo[d]imidazole-6-carboxamide |
| 29 CDD-2619 | 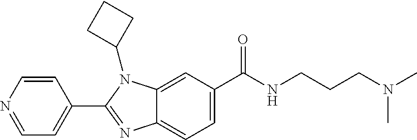 [SMILES: CN(C)CCCNC(=O)c1ccc2nc(-c3ccncc3)n(C3CCC3)c2c1] |
| | 1-cyclobutyl-N-(3-(dimethylamino)propyl)-2-(pyridin-4-yl)-1H- |
| | benzo[d]imidazole-6-carboxamide |
| 30 CDD-2620 | 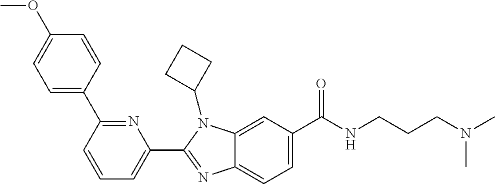 |
| | 1-cyclobutyl-N-(3-(dimethylamino)propyl)-2-(6-(4-methoxyphenyl)pyridin-2-yl)- |
| | 1H-benzo[d]imidazole-6-carboxamide |
| 31 CDD-2621 | 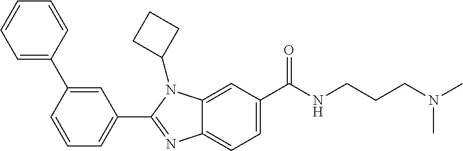 [SMILES: CN(C)CCCNC(=O)c1ccc2nc(-c3cccc(-c4ccccc4)c3)n(C3CCC3)c2c1] |
| | 2-([1,1′-biphenyl]-3-yl)-1-cyclobutyl-N-(3-(dimethylamino)propyl)-1H- |
| | benzo[d]imidazole-6-carboxamide |
| 32 CDD-2622 | 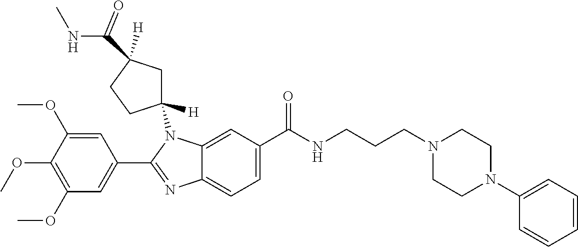 [SMILES: CNC(=O)[C@@H]1CC[C@@H](n2c(-c3cc(OC)c(OC)c(OC)c3)nc3ccc(C(=O)NCCCN4CCN(c5ccccc5)CC4)cc32)C1] |
| | 1-((1R,3R)-3-(methylcarbamoyl)cyclopentyl)-N-(3-(4-phenylpiperazin-1- |
| | yl)propyl)-2-(3,4,5-trimethoxyphenyl)-1H-benzo[d]imidazole-6-carboxamide |
| 33 CDD-2623 | 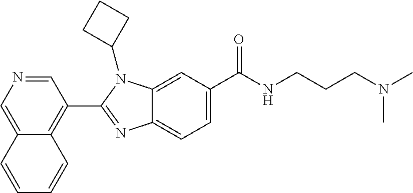 [SMILES: CN(C)CCCNC(=O)c1ccc2nc(-c3cncc4ccccc34)n(C3CCC3)c2c1] |
| | 1-cyclobutyl-N-(3-(dimethylamino)propyl)-2-(isoquinolin-4-yl)-1H- |
| | benzo[d]imidazole-6-carboxamide |
| 34 CDD-2624 | 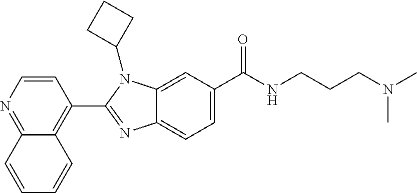 [SMILES: CN(C)CCCNC(=O)c1ccc2nc(-c3ccnc4ccccc34)n(C3CCC3)c2c1] |
| | 1-cyclobutyl-N-(3-(dimethylamino)propyl)-2-(quinolin-4-yl)-1H- |
| | benzo[d]imidazole-6-carboxamide |
| 35 CDD-2625 | 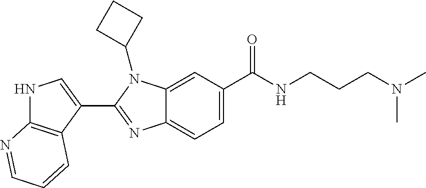 [SMILES: CN(C)CCCNC(=O)c1ccc2nc(-c3c[nH]c4ncccc34)n(C3CCC3)c2c1] |
| | 1-cyclobutyl-N-(3-(dimethylamino)propyl)-2-(1H-pyrrolo[2,3-b]pyridin-3-yl)- |
| | 1H-benzo[d]imidazole-6-carboxamide |
| 36 CDD-2682 | 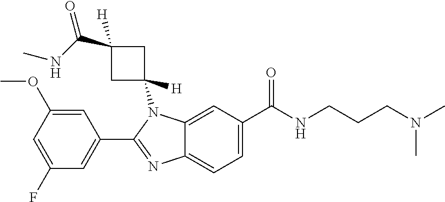 |
| | N-(3-(dimethylamino)propyl)-2-(3-fluoro-5-methoxyphenyl)-1-((1r,3r)-3- |
| | (methylcarbamoyl)cyclobutyl)-1H-benzo[d]imidazole-6-carboxamide |
| 37 CDD-2683 | 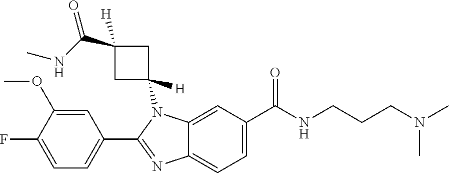 |
| | N-(3-(dimethylamino)propyl)-2-(4-fluoro-3-methoxyphenyl)-1-((1r,3r)-3- |
| | (methylcarbamoyl)cyclobutyl)-1H-benzo[d]imidazole-6-carboxamide |
| 38 CDD-2684 | 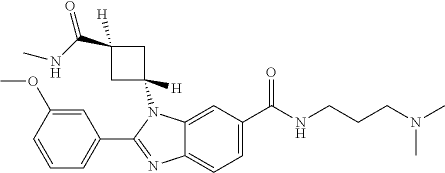 |
| | N-(3-(dimethylamino)propyl)-2-(3-methoxyphenyl)-1-((1r,3r)-3- |
| | (methylcarbamoyl)cyclobutyl)-1H-benzo[d]imidazole-6-carboxamide |
| 39 CDD-2685 | 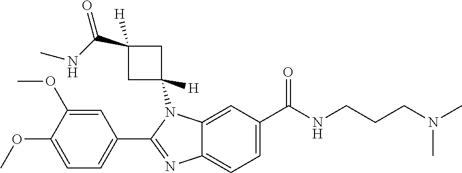 |
| | 2-(3,4-dimethoxyphenyl)-N-(3-(dimethylamino)propyl)-1-((1r,3r)-3- |
| | (methylcarbamoyl)cyclobutyl)-1H-benzo[d]imidazole-6-carboxamide |
| 40 CDD-2686 | 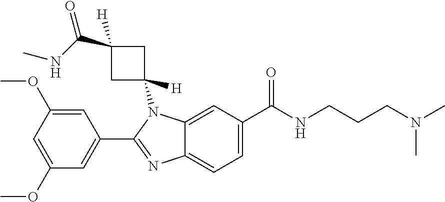 |
| | 2-(3,5-dimethoxyphenyl)-N-(3-(dimethylamino)propyl)-1-((1r,3r)-3- |
| | (methylcarbamoyl)cyclobutyl)-1H-benzo[d]imidazole-6-carboxamide |
| 41 CDD-2687 | 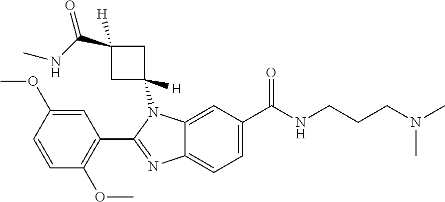 |
| | 2-(2,5-dimethoxyphenyl)-N-(3-(dimethylamino)propyl)-1-((1r,3r)-3- |
| | (methylcarbamoyl)cyclobutyl)-1H-benzo[d]imidazole-6-carboxamide |
| 42 CDD-2786 | 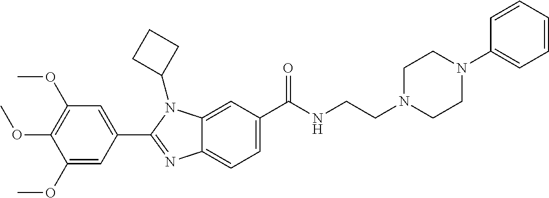 [SMILES: COc1cc(-c2nc3ccc(C(=O)NCCN4CCN(c5ccccc5)CC4)cc3n2C2CCC2)cc(OC)c1OC] |
| | 1-cyclobutyl-N-(2-(4-phenylpiperazin-1-yl)ethyl)-2-(3,4,5-trimethoxyphenyl)- |
| | 1H-benzo[d]imidazole-6-carboxamide |
| 43 CDD-2787 | 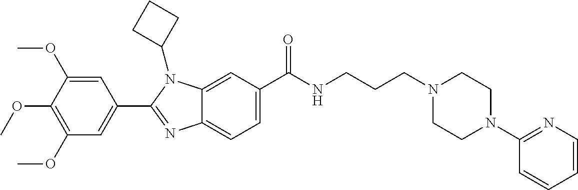 [SMILES: COc1cc(-c2nc3ccc(C(=O)NCCCN4CCN(c5ccccn5)CC4)cc3n2C2CCC2)cc(OC)c1OC] |
| | 1-cyclobutyl-N-(3-(4-(pyridin-2-yl)piperazin-1-yl)propyl)-2-(3,4,5- |
| | trimethoxyphenyl)-1H-benzo[d]imidazole-6-carboxamide |
| 44 CDD-2788 | 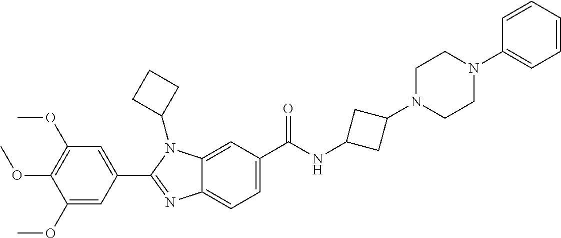 [SMILES: COc1cc(-c2nc3ccc(C(=O)NC4CC(N5CCN(c6ccccc6)CC5)C4)cc3n2C2CCC2)cc(OC)c1OC] |
| | 1-cyclobutyl-N-(3-(4-phenylpiperazin-1-yl)cyclobutyl)-2-(3,4,5- |
| | trimethoxyphenyl)-1H-benzo[d]imidazole-6-carboxamide |
| 45 CDD-2789 | 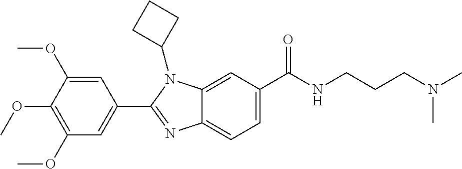 |
| | 1-cyclobutyl-N-(3-(dimethylamino)propyl)-2-(3,4,5-trimethoxyphenyl)-1H- |
| | benzo[d]imidazole-6-carboxamide |
| 46 CDD-2790 | 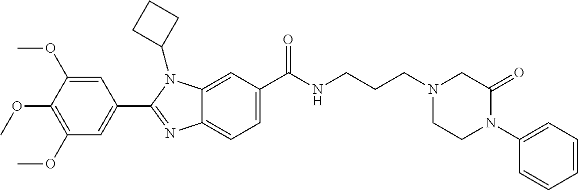 [SMILES: COc1cc(-c2nc3ccc(C(=O)NCCCN4CCN(c5ccccc5)C(=O)C4)cc3n2C2CCC2)cc(OC)c1OC] |
| | 1-cyclobutyl-N-(3-(3-oxo-4-phenylpiperazin-1-yl)propyl)-2-(3,4,5- |
| | trimethoxyphenyl)-1H-benzo[d]imidazole-6-carboxamide |
| 47 CDD-2791 | 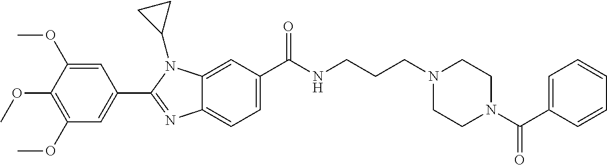 [SMILES: COc1cc(-c2nc3ccc(C(=O)NCCCN4CCN(C(=O)c5ccccc5)CC4)cc3n2C2CC2)cc(OC)c1OC] |
| | N-(3-(4-benzoylpiperazin-1-yl)propyl)-1-cyclobutyl-2-(3,4,5-trimethoxyphenyl)- |
| | 1H-benzo[d]imidazole-6-carboxamide |
| 48 CDD-2792 | 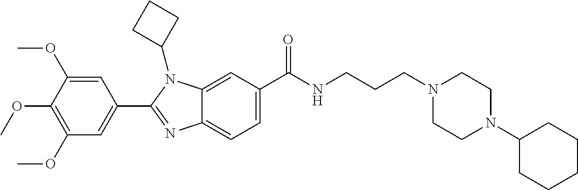 [SMILES: COc1cc(-c2nc3ccc(C(=O)NCCCN4CCN(C5CCCCC5)CC4)cc3n2C2CCC2)cc(OC)c1OC] |
| | 1-cyclobutyl-N-(3-(4-cyclohexylpiperazin-1-yl)propyl)-2-(3,4,5- |
| | trimethoxyphenyl)-1H-benzo[d]imidazole-6-carboxamide |
| 49 CDD-2793 | 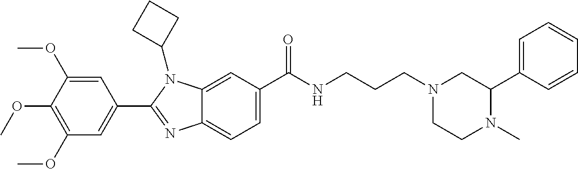 [SMILES: COc1cc(-c2nc3ccc(C(=O)NCCCN4CCN(C)C(c5ccccc5)C4)cc3n2C2CCC2)cc(OC)c1OC] |
| | 1-cyclobutyl-N-(3-(4-methyl-3-phenylpiperazin-1-yl)propyl)-2-(3,4,5- |
| | trimethoxyphenyl)-1H-benzo[d]imidazole-6-carboxamide |
| 50 CDD-2794 | 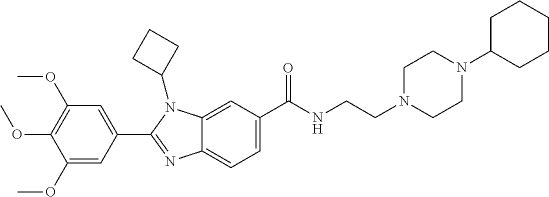 [SMILES: COc1cc(-c2nc3ccc(C(=O)NCCN4CCN(C5CCCCC5)CC4)cc3n2C2CCC2)cc(OC)c1OC] |
| | 1-cyclobutyl-N-(2-(4-cyclohexylpiperazin-1-yl)ethyl)-2-(3,4,5- |
| | trimethoxyphenyl)-1H-benzo[d]imidazole-6-carboxamide |
| 51 CDD-2795 | 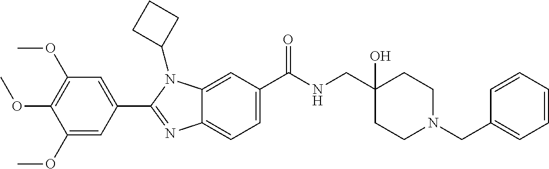 [SMILES: COc1cc(-c2nc3ccc(C(=O)NCC4(O)CCN(Cc5ccccc5)CC4)cc3n2C2CCC2)cc(OC)c1OC] |
| | N-((1-benzyl-4-hydroxypiperidin-4-yl)methyl)-1-cyclobutyl-2-(3,4,5- |
| | trimethoxyphenyl)-1H-benzo[d]imidazole-6-carboxamide |

In another aspect, the present disclosure provides an antibody-drug conjugate (ADC) comprising at least one compound of the present disclosure and an antibody, wherein the compound is covalently conjugated to the antibody.

In certain embodiments, the antibody specifically binds to AMHR2. In certain embodiments, the ADC is suitable for delivery of the compound to at least one cell type selected from the group consisting of ovarian granulosa cells and endometrioma cells.

### Methods

In another aspect, the present disclosure provides a method of treating, preventing, and/or ameliorating a disease or disorder in a subject in need thereof. In certain embodiments, the method comprises administering to the subject a therapeutically effective amount of at least one compound of the present disclosure, at least one ADC of the present disclosure, and/or at least one pharmaceutical composition of the present disclosure.

In certain embodiments, the disease or disorder is characterized by one or more ACVR1 activating mutations or overactive ACVR1.

In certain embodiments, the disease or disorder is at least one selected from the group consisting of fibrodysplasia ossificans progressiva (FOP), atherosclerosis, anemia, iron deficiency, tumor angiogenesis and/or vasculogenesis, and cancer.

In certain embodiments, the cancer is at least one selected from the group consisting of diffuse intrinsic pontine glioma (DIPG), ependymoma, breast cancer, endometrial cancer, and pancreatic cancer.

In certain embodiments, the subject is further administered at least one additional therapeutic agent.

In another aspect, the present disclosure provides a method of inhibiting ACVR1 in a subject need in thereof, the method comprising administering to the subject a therapeutically effective amount of at least one compound of the present disclosure, at least one ADC of the present disclosure, and/or at least one pharmaceutical composition of the present disclosure.

In certain embodiments, the ACVR1 is ACVR1^{WT}. In certain embodiments, the ACVR1 is an ACVR1 mutant comprising one or more activating mutations.

In another aspect, the present disclosure provides a method of inhibiting and/or reducing activity of ACVRL1 in a subject in need thereof, the method comprising administering to the subject a therapeutically effective amount of at least one compound of the present disclosure, at least one ADC of the present disclosure, and/or at least one pharmaceutical composition of the present disclosure.

In certain embodiments, the ACVRL1 is an ACVRL1 mutant comprising one or more activating mutations. In certain embodiments, the ACVRL1 is an ACVRL1^{WT}.

In certain embodiments, the subject is further administered at least one additional therapeutic agent.

In certain embodiments, the subject is a mammal. In certain embodiments, the mammal is a human.

In certain embodiments, the compounds of the present disclosure are suitable for inhibition of ACVR1 receptor mediated events associated with genetic mutations in ACVR1 (e.g., for FOP and/or DIPG) that cause a change in the normal function of the ACVR1 receptor.

In certain embodiments, the compounds of the present disclosure are suitable for inhibition of ACVR1-mediated and ACVRL1-mediated events associated with trauma-related heterotopic ossification (e.g., blast injury).

In certain embodiments, the compounds of the present disclosure are suitable for inhibition of wild-type ACVR1 (i.e., ACVR1^{WT}) receptor-mediated events that are misdirected by chronic inflammation into disease mechanisms that promote atherogenesis, iron-deficient anemia, chronic kidney disease, growth and survival of endometrial tumors, pancreatic cancer, breast cancer, other cancers, tumor vasculogenesis, and Sjogren's syndrome.

In certain embodiments, the compounds of the present disclosure are suitable for reduction of the adverse effects of rapamycin, tacrolimus or similar calcineurin regulators used as part of immunosuppression therapies on hepcidin regulation and balance in immunosuppressed patients.

In certain embodiments, the compounds of the present disclosure are suitable for inhibition of ACVRL1 receptor-mediated events in Kupfer cells and macrophages during inflammation through either or both of TGFβ and BMP9 mechanisms that are dependent or independent of the endoglin receptor. In certain embodiments, inhibition of ACVRL1 is predicted to reduce tumor-induced angiogenesis, resulting in leaky blood vessels that are a part of triggers for immunosuppression and would result in reduced tumor growth, while sustaining normal vascular integrity.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

The use of antibodies to deliver therapeutic proteins in the form of antibody drug conjugates is a technology to deliver drugs to specific tissues. For example, an antibody to the AMHR2 could be used to deliver an ACVR1 inhibitor to ovarian cells or to disease tissue (e.g., endometriosis tissue) or to cancers (e.g., ovarian cancer) with expression or overexpression of AMHR2. In this context, an antibody bound to the cell surface AMHR2 transmembrane receptor is internalized as a consequence of the constitutive recycling of these type 2 receptors. In the same method, antibodies that specifically target ACVR2A or ACVR2B or BMPR2 could be used to achieve cell-specific delivery of ACVR1 inhibitors to avoid systemic exposure to this inhibitor when cell-specific delivery is the preferred therapeutic objective. The antibody form utilized can represent full length or modified immunoglobulin designs incorporating IgG1, IgG4 or fragments referred to as single chain Fv (scFv) or diabody or similar modifications of an antibody that are specifically utilized for targeted delivery of small molecules without the need for antibody dependent complement cascade (ADCC) or antibody dependent phagocytosis cascade (ADPC).

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors. The skilled artisan is similarly able to determine appropriate dosages for antibody-drug conjugates, based on the half-life and daily maximum exposure achievable with the antibody, or antibody fragments, selected for targeted delivery of an ACVR1 inhibitor.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

Various embodiments of the present application can be better understood by reference to the following Examples which are offered by way of illustration. The scope of the present application is not limited to the Examples given herein.

### Example 1: DNA-Encoded Library Screening

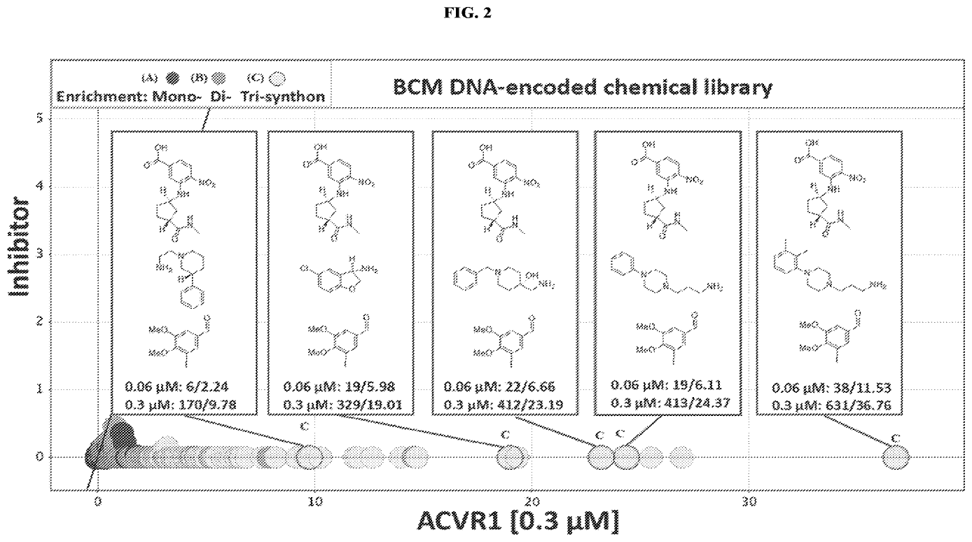

To identify ACVR1-specific molecules for the treatment of patients with FOP, DIPG, and other diseases, a DNA-encoded chemistry technology (DEC-Tec) platform was used to perform parallel selections against 4.54 billion DNA-encoded small molecules (45 libraries) using a His-tagged ACVR1 kinase domain, with and without kinase competitive inhibitor (i.e., LDN-193189 at 20 μM), and in the absence of ACVR1 (i.e., control). A series of hits were identified in library qDOS18_2 with excellent structure-enrichment relationships (SER) that bind ACVR1, share common building blocks, and are competitive with LDN-193189 (FIG. 2).

### Example 2: Compound Synthesis

### General Synthetic Procedure

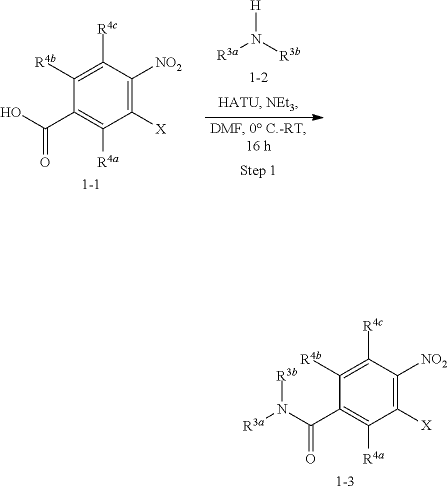

Step 1: In a round bottom flask equipped with a magnetic stir bar and septum under nitrogen, acid compound 1-1 (1.0 equiv.) was dissolved in DMF, then amine 1-2 (1.0 equiv.) was added, followed by HATU (1.2 equiv.) and NEt₃ (1.5 equiv.) at 0° C., and the mixture was allowed to warm to room temperature. The reaction was allowed to stir for 16 h, after which time TLC and LCMS indicated complete consumption of starting material. The reaction was diluted with ethyl acetate and washed with sat. aq NaHCO₃ and brine. The organic phase was collected and dried over anhydrous Na₂SO₄, filtered, and concentrated reduced pressure to give the crude product which was purified by silica gel chromatography (ethyl acetate/hexanes) to provide the desired product 1-3.

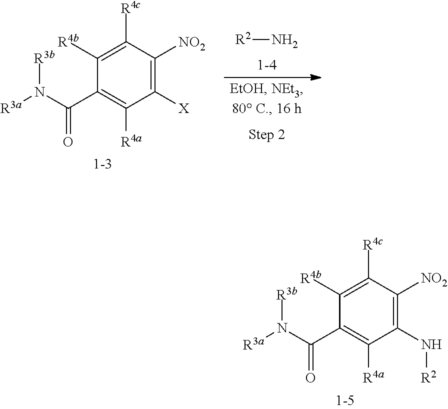

Step 2: In a round bottom flask equipped with a magnetic stir bar and septum under nitrogen, compound 1-3 (1 equiv.) was dissolved in ethanol, then amine 1-4 (1.2 equiv.) and NEt₃ (1.5 equiv.) were added at RT. The reaction was allowed to stir at 80° C. for 16 h, after which time TLC and LCMS indicated complete consumption of starting material. Then the solvent was removed under reduced pressure to give the crude residue which was purified by silica gel chromatography (ethyl acetate/hexanes) to provide the desired product 1-5.

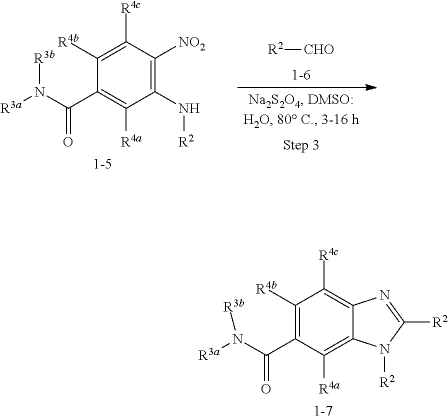

Step 3: In a round bottom flask equipped with magnetic stir bar and septum under nitrogen, compound 1-5 (1 equiv.) was dissolved in DMSO:water (5:1), then aldehyde 1-6 (1.3 equiv.) was added followed by Na₂S₂O₄ (6.0 equiv.) at room temperature. The reaction was allowed to stir for 3-16 h at 80° C., after which time TLC and LCMS indicated complete consumption of starting material. The reaction was diluted with ethyl acetate and washed with sat. aq NaHCO₃ and brine. The organic phase was collected and dried over anhydrous Na₂SO₄, filtered and concentrated reduced pressure to give the crude product which was purification by silica gel column chromatography (methanol/dichloromethane) provided the desired product 1-7.

The following intermediate compounds were prepared according step 1 of the general procedure described herein:

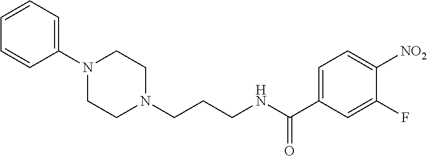 [SMILES: O=C(NCCCN1CCN(c2ccccc2)CC1)c1ccc([N+](=O)[O-])c(F)c1]

### 3-fluoro-4-nitro-N-(3-(4-phenylpiperazin-1-yl)propyl)benzamide

Molecular Formula: C₂₀H₂₃FN₄O₃; yield 90%, white solid. ¹H NMR (600 MHz, CDCl₃) δ ppm 8.62 (s, 1H), 8.02 (dd, J=8.2, 7.4 Hz, 1H), 7.77 (dd, J=11.1, 1.6 Hz, 1H), 7.69 (d, J=8.5 Hz, 1H), 7.32-7.27 (m, 2H), 6.91 (dd, J=7.6, 4.9 Hz, 3H), 3.62 (dd, J=10.9, 5.7 Hz, 2H), 3.21-3.16 (m, 4H), 2.72-2.69 (m, 4H), 2.68-2.64 (m, 2H), 1.89-1.83 (m, 2H). ¹³C NMR (150 MHz, CDCl₃) δ ppm 163.9, 156.2, 154.5, 150.9, 141.7, 141.7, 138.8, 138.7, 129.3, 126.4, 126.4, 122.9, 122.9, 120.5, 117.7, 117.6, 116.4, 58.1, 53.4, 49.4, 41.1, 23.9. LCMS m/z calcd for (M+H)⁺ 387.4, found 387.4.

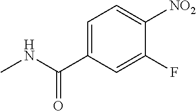

### 3-fluoro-N-methyl-4-nitrobenzamide

Molecular Formula: C₈H₇FN₂O₃; yield 56%, white solid. ¹H NMR (600 MHz, CDCl₃) δ ppm 8.12 (dd, J=8.2, 7.4 Hz, 1H), 7.72 (dd, J=11.0, 1.6 Hz, 1H), 7.64 (d, J=8.5 Hz, 1H), 6.24 (s, 1H), 3.05 (d, J=4.9 Hz, 3H). LCMS m/z calcd for (M+H)⁺ 199.1, found 199.2.

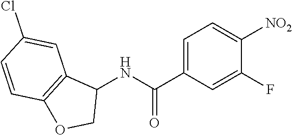 [SMILES: O=C(NC1COc2ccc(Cl)cc21)c1ccc([N+](=O)[O-])c(F)c1]

### N-(5-chloro-2,3-dihydrobenzofuran-3-yl)-3-fluoro-4-nitrobenzamide

Molecular Formula: C₁₅H₁₀ClFN₂O₄; yield 88%, white solid. ¹H NMR (600 MHz, CDCl₃) δ ppm 8.12 (dd, J=8.4, 7.2 Hz, 1H), 7.74 (dd, J=10.8, 1.7 Hz, 1H), 7.65 (d, J=8.4 Hz, 1H), 7.36 (d, J=2.0 Hz, 1H), 7.25 (d, J=2.2 Hz, 1H), 6.85 (d, J=8.6 Hz, 1H), 6.48 (d, J=6.5 Hz, 1H), 5.76 (d, J=3.4 Hz, 1H), 4.82 (dd, J=10.4, 7.8 Hz, 1H), 4.49 (dd, J=10.4, 3.5 Hz, 1H); ¹³C NMR (150 MHz, CDCl₃) δ ppm 163.8, 159.4, 140.2, 131.0, 127.1, 126.8, 126.3, 125.6, 122.9, 118.1, 117.9, 111.9, 78.3, 52.6. LCMS m/z calcd for (M+H)⁺ 337.7, found 337.8.

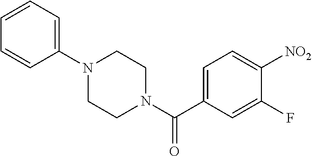 [SMILES: O=C(c1ccc([N+](=O)[O-])c(F)c1)N1CCN(c2ccccc2)CC1]

### (3-fluoro-4-nitrophenyl) (4-phenylpiperazin-1-yl) methanone

Molecular Formula: C₁₇H₁₆FN₃O₃; yield 85%, white solid. ¹H NMR (600 MHz, CDCl₃) δ ppm 8.17-8.11 (m, 1H), 7.40-7.35 (m, 2H), 7.33-7.28 (m, 2H), 6.97-6.92 (m, 3H), 3.95 (bs, 2H), 3.55 (bs, 2H), 3.28 (bs, 2H), 3.14 (bs, 2H). ¹³C NMR (150 MHz, CDCl₃) δ ppm 166.5, 156.3, 154.6, 150.7, 142.9, 142.9, 129.4, 126.7, 126.7, 123.2, 123.1, 121.1, 117.6, 117.4, 116.9, 50.0, 49.5, 47.5, 42.3. LCMS m/z calcd for (M+H)⁺ 330.3, found 330.4.

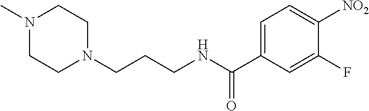 [SMILES: CN1CCN(CCCNC(=O)c2ccc([N+](=O)[O-])c(F)c2)CC1]

### 3-fluoro-N-(3-(4-methylpiperazin-1-yl)propyl)-4-nitrobenzamide

Molecular Formula: C₁₅H₂₁FN₄O₃; yield 42%, colorless oil. ¹H NMR (600 MHz, CDCl₃) δ ppm 8.94 (s, 1H), 8.12 (dd, J=8.6, 7.3 Hz, 1H), 7.81-7.76 (m, 2H), 3.59 (dd, J=10.5, 5.6 Hz, 2H), 2.61 (dd, J=19.5, 13.9 Hz, 5H), 2.42 (bs, 4H), 2.31 (bs, 4H), 1.80 (dd, J=11.2, 5.7 Hz, 2H). ¹³C NMR (150 MHz, CDCl₃) δ ppm 163.7, 163.7, 156.2, 154.4, 141.8, 141.8, 138.8, 138.7, 126.3, 123.3, 117.4, 58.7, 55.3, 53.4, 46.1, 41.8, 23.5. LCMS m/z calcd for (M+H)⁺ 325.3, found 325.4.

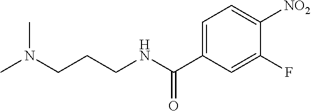 [SMILES: CN(C)CCCNC(=O)c1ccc([N+](=O)[O-])c(F)c1]

### N-(3-(dimethylamino)propyl)-3-fluoro-4-nitrobenzamide

Molecular Formula: C₁₂H₁₆FN₃O₃; yield 38%, colorless oil. ¹H NMR (600 MHz, CDCl₃) δ ppm 9.27 (s, 1H), 8.10 (dd, J=8.2, 7.5 Hz, 1H), 7.77 (d, J=11.3 Hz, 1H), 7.73 (d, J=8.5 Hz, 1H), 3.61 (d, J=5.8 Hz, 2H), 2.73-2.67 (m, 2H), 2.45 (bs, 6H), 1.92-1.83 (m, 2H). ¹³C NMR (150 MHz, CDCl₃) δ ppm 163.6, 156.4, 154.7, 141.8, 138.8, 126.5, 126.5, 122.8, 117.6, 117.5, 59.5, 45.3, 41.3, 24.3. LCMS m/z calcd for (M+H)⁺ 270.2, found 270.3.

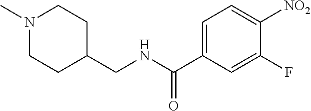 [SMILES: CN1CCC(CNC(=O)c2ccc([N+](=O)[O-])c(F)c2)CC1]

### 3-fluoro-N-((1-methylpiperidin-4-yl)methyl)-4-nitrobenzamide

Molecular Formula: C₁₄H₁₈FN₃O₃; yield 63%, colorless oil. ¹H NMR (600 MHz, CDCl₃) δ ppm 8.06-8.02 (m, 1H), 7.78 (d, J=11.1 Hz, 1H), 7.73 (d, J=8.4 Hz, 1H), 3.33 (t, J=6.2 Hz, 2H), 3.07 (d, J=11.1 Hz, 2H), 2.41 (s, 3H), 2.21 (t, J=11.0 Hz, 2H), 1.80 (t, J=19.1 Hz, 3H), 1.58 (dd, J=22.4, 10.9 Hz, 2H). ¹³C NMR (150 MHz, CDCl₃) δ ppm 164.6, 156.4, 154.6, 141.4, 126.5, 123.2, 123.2, 118.0, 117.9, 55.2, 45.4, 38.8, 34.4, 28.9. LCMS m/z calcd for (M+H)⁺296.3, found 296.3.

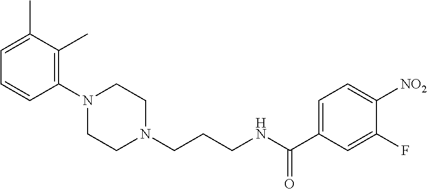 [SMILES: Cc1cccc(N2CCN(CCCNC(=O)c3ccc([N+](=O)[O-])c(F)c3)CC2)c1C]

### N-(3-(4-(2,3-dimethylphenyl)piperazin-1-yl)propyl)-3-fluoro-4-nitrobenzamide

Molecular Formula: C₂₂H₂₇FN₄O₃; yield 83%, colorless oil. ¹H NMR (600 MHz, CDCl₃) δ ppm 8.99 (bs, 1H), 8.13-8.09 (m, 1H), 7.83 (dd, J=11.1, 1.2 Hz, 1H), 7.79 (d, J=8.5 Hz, 1H), 7.12 (t, J=7.7 Hz, 1H), 6.94 (d, J=7.5 Hz, 1H), 6.86 (d, J=8.0 Hz, 1H), 3.63 (d, J=5.6 Hz, 2H), 2.92 (bs, 4H), 2.73 (bs, 4H), 2.27 (s, 3H), 2.21 (s, 3H), 1.91-1.84 (m, 2H). ¹³C NMR (150 MHz, CDCl₃) δ ppm 163.9, 156.4, 154.6, 150.9, 141.9, 141.9, 138.9, 138.9, 138.3, 131.4, 126.4, 126.4, 126.2, 125.7, 123.2, 123.2, 117.8, 117.6, 116.5, 58.7, 54.0, 52.2, 38.7, 23.6, 20.7, 13.9. LCMS m/z calcd for (M+H)⁺ 415.4, found 415.5.

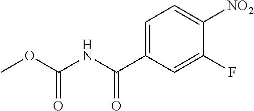 [SMILES: COC(=O)NC(=O)c1ccc([N+](=O)[O-])c(F)c1]

### Methyl 3-(3-fluoro-4-nitrobenzamido) propanoate

Molecular Formula: C₁₁H₁₁FN₂₀₅; yield 50%, white solid. ¹H NMR (600 MHz, CDCl₃) δ ppm 8.11 (t, J=7.5 Hz, 1H), 7.74 (d, J=10.9 Hz, 1H), 7.66 (d, J=8.3 Hz, 1H), 7.17 (s, 1H), 3.73 (d, J=6.5 Hz, 5H), 2.72-2.65 (m, 2H). LCMS m/z calcd for (M+H)⁺ 271.2, found 271.2.

The following intermediate compounds were prepared according step 2 of the general procedure described herein:

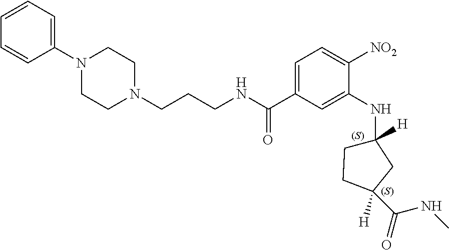

### 3-(((1S,3S)-3-(methylcarbamoyl)cyclopentyl)amino)-4-nitro-N-(3-(4-phenylpiperazin-1-yl)propyl)benzamide

Molecular Formula: C₂₇H₃₆N₆O₄; yield 100%, yellow liquid. ¹H NMR (600 MHz, CDCl₃) δ ppm 8.21 (d, J=13.1 Hz, 1H), 8.16 (d, J=8.8 Hz, 1H), 8.05 (d, J=6.3 Hz, 1H), 7.45 (s, 1H), 7.29 (dd, J=8.4, 7.5 Hz, 2H), 7.04 (d, J=8.5 Hz, 1H), 6.94 (t, J=7.3 Hz, 1H), 6.90 (d, J=7.9 Hz, 2H), 5.66 (s, 1H), 4.30 (dd, J=11.2, 5.7 Hz, 1H), 3.61 (dd, J=11.8, 5.5 Hz, 2H), 3.38 (s, 4H), 3.03 (d, J=17.6 Hz, 5H), 2.81 (d, J=4.8 Hz, 4H), 2.43-2.30 (m, 2H), 2.07 (dd, J=8.0, 4.9 Hz, 3H), 1.99-1.84 (m, 3H), 1.65 (dd, J=13.3, 5.5 Hz, 1H). LCMS m/z calcd for (M+H)⁺ 509.6, found 509.6.

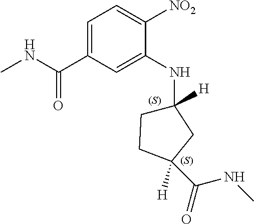

### N-methyl-3-(((1S,3S)-3-(methylcarbamoyl)cyclopentyl)amino)-4-nitrobenzamide

Molecular Formula: C₁₅H₂₀N₄O₄; yield 62%, yellow solid. ¹H NMR (600 MHz, CD₃OD) δ 8.20 (d, J=8.9 Hz, 1H), 7.44 (d, J=1.6 Hz, 1H), 7.04 (dd, J=8.9, 1.8 Hz, 1H), 4.34-4.27 (m, 1H), 2.96-2.93 (m, 4H), 2.75 (s, 3H), 2.42-2.30 (m, 2H), 2.11 (dt, J=11.9, 4.1 Hz, 1H), 1.95-1.87 (m, 2H), 1.73-1.71 (m, 1H). ¹³C NMR (150 MHz, CD₃OD) δ ppm 178.6, 169.1, 145.7, 142.5, 134.3, 128.1, 115.1, 114.4, 55.2, 44.5, 38.1, 34.7, 29.7, 27.0, 26.4. LCMS m/z calcd for (M+H)⁺ 385.4, found 385.3.

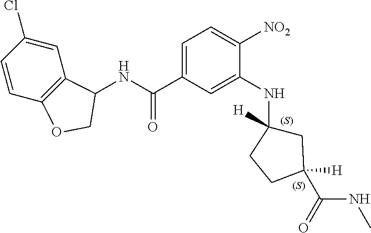

### N-(5-chloro-2,3-dihydrobenzofuran-3-yl)-3-(((1S,3S)-3-(methylcarbamoyl)cyclo pentyl)amino)-4-nitrobenzamide

Molecular Formula: C₂H₂₃ClN₄O₅; yield 62%, yellow liquid. ¹H NMR (600 MHz, CDCl₃) δ ppm 8.20 (d, J=8.8 Hz, 1H), 8.07 (d, J=5.4 Hz, 1H), 7.42 (s, 1H), 7.37 (d, J=2.0 Hz, 1H), 7.25-7.22 (m, 1H), 6.89 (dd, J=8.8, 1.7 Hz, 1H), 6.83 (d, J=8.6 Hz, 1H), 6.68 (s, 1H), 5.81-5.77 (m, 1H), 5.52 (s, 1H), 4.84-4.80 (m, 1H), 4.50 (dd, J=10.3, 3.9 Hz, 1H), 4.25 (d, J=5.4 Hz, 1H), 3.12 (dd, J=7.3, 4.8 Hz, 2H), 2.83 (d, J=4.8 Hz, 3H), 2.79 (d, J=7.6 Hz, 1H), 2.45-2.41 (m, 1H), 2.36 (m, 1H), 2.25 (m, 1H), 2.11 (d, J=8.1 Hz, 1H), 1.95 (m, 1H), 1.84 (m, 1H). LCMS m/z calcd for (M+H)⁺ 524.0, found 524.0.

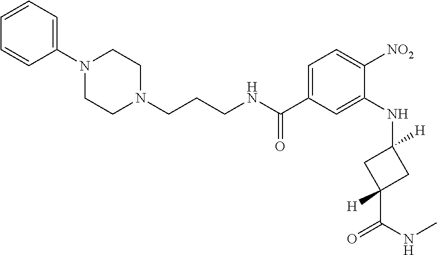 [SMILES: CNC(=O)[C@H]1C[C@H](Nc2cc(C(=O)NCCCN3CCN(c4ccccc4)CC3)ccc2[N+](=O)[O-])C1]

### 3-(((1r,3r)-3-(methylcarbamoyl)cyclobutyl)amino)-4-nitro-N-(3-(4-phenylpiperazin-1-yl)propyl)benzamide

Molecular Formula: C₂₆H₃₄N₆O₄; yield 100%, yellow solid. ¹H NMR (600 MHz, CD₃OD) δ ppm 8.19 (d, J=8.8 Hz, 1H), 7.26 (dd, J=8.5, 7.5 Hz, 2H), 7.23 (d, J=1.5 Hz, 1H), 7.07 (dd, J=8.8, 1.7 Hz, 1H), 6.99 (d, J=8.0 Hz, 2H), 6.89 (t, J=7.3 Hz, 1H), 4.44-4.39 (m, 1H), 3.50 (t, J=6.7 Hz, 2H), 3.34 (bs, 4H), 3.12 (dd, J=8.5, 4.4 Hz, 5H), 2.96 (bs, 2H), 2.76-2.72 (m, 5H), 2.30-2.32 (m, 2H), 2.06-1.98 (m, 2H). ¹³C NMR (150 MHz, CD₃OD) δ ppm 178.1, 169.0, 151.8, 145.0, 142.3, 134.6, 130.2, 128.0, 121.9, 117.8, 115.0, 114.8, 56.4, 47.3, 38.8, 35.4, 34.2, 26.5, 26.2. LCMS m/z calcd for (M+H)⁺ 495.6, found 495.6.

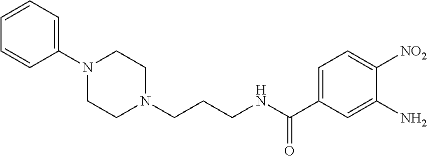 [SMILES: Nc1cc(C(=O)NCCCN2CCN(c3ccccc3)CC2)ccc1[N+](=O)[O-]]

### 3-amino-4-nitro-N-(3-(4-phenylpiperazin-1-yl)propyl)benzamide

Molecular Formula: C₂₀H₂₅N₅O₃; yield 87%, yellow solid. ¹H NMR (600 MHz, DMSO-d₆) δ ppm 8.62 (t, J=5.2 Hz, 1H), 8.01 (d, J=8.9 Hz, 1H), 7.51 (s, 2H), 7.46 (d, J=1.0 Hz, 1H), 7.20 (t, J=7.8 Hz, 2H), 6.97 (dd, J=8.9, 1.2 Hz, 1H), 6.92 (d, J=8.2 Hz, 2H), 6.77 (t, J=7.2 Hz, 1H), 3.31-3.28 (m, 2H), 3.12 (bs, 4H), 2.52 (d, J=5.7 Hz, 4H), 2.39 (t, J=6.9 Hz, 2H), 1.75-1.67 (m, 2H). ¹³C NMR (150 MHz, DMSO-d₆) δ ppm 165.1, 151.0, 145.8, 141.1, 131.1, 128.9, 125.7, 118.8, 118.7, 115.3, 113.3, 55.5, 52.7, 48.2, 37.9, 26.1. LCMS m/z calcd for (M+H)⁺ 384.4, found 384.4.

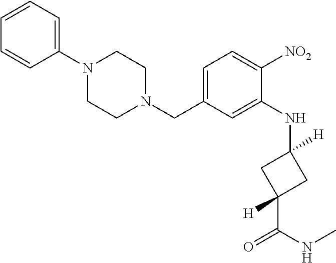 [SMILES: CNC(=O)[C@H]1C[C@H](Nc2cc(CN3CCN(c4ccccc4)CC3)ccc2[N+](=O)[O-])C1]

### (1r,3r)-N-methyl-3-((2-nitro-5-(4-phenylpiperazine-1-carbonyl)phenyl)amino)cyclo butane-1-carboxamide

Molecular Formula: C₂₃H₂₇N₅O₄; yield 100%, yellow solid. ¹H NMR (600 MHz, CDCl₃) δ ppm 8.21 (d, J=8.6 Hz, 1H), 8.16 (d, J=5.5 Hz, 1H), 7.30 (t, J=7.8 Hz, 2H), 7.06-6.90 (m, 3H), 6.73 (s, 1H), 6.65 (dd, J=8.6, 1.2 Hz, 1H), 5.62 (s, 1H), 4.41 (dd, J=13.5, 6.8 Hz, 1H), 3.96 (bs, 2H), 3.56 (s, 2H), 3.30 (s, 2H), 3.13 (s, 2H), 3.01-2.96 (m, 1H), 2.82 (dd, J=10.8, 4.3 Hz, 3H), 2.82-2.77 (m, 3H), 2.25 (dt, J=12.5, 9.6 Hz, 2H). ¹³C NMR (150 MHz, CDCl₃) δ ppm 175.2, 168.5, 144.3, 143.3, 132.3, 129.6, 127.7, 117.5, 113.7, 112.9, 50.8, 47.1, 46.2, 41.8, 38.7, 34.7, 33.7, 29.8, 26.6. LCMS m/z calcd for (M+H)⁺ 338.5, found 338.4.

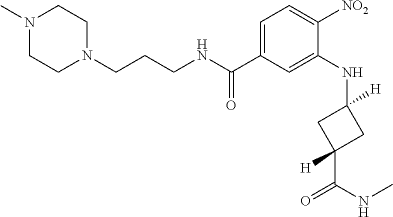

### 3-(((1r,3r)-3-(methylcarbamoyl)cyclobutyl)amino)-N-(3-(4-methylpiperazin-1-yl) propyl)-4-nitrobenzamide

Molecular Formula: C₂₁H₃₂N₆O₄; yield 77%, yellow liquid. ¹H NMR (600 MHz, CDCl₃) δ ppm 8.36 (bs, 1H), 8.19 (d, J=8.8 Hz, 1H), 8.13 (d, J=5.5 Hz, 1H), 7.25 (bs, 1H), 7.06 (d, J=8.7 Hz, 1H), 5.97 (bs, 1H), 4.51-4.40 (m, 1H), 3.57 (dd, J=11.7, 5.7 Hz, 2H), 3.03 (dd, J=9.4, 4.8 Hz, 1H), 2.86 (dd, J=10.1, 4.7 Hz, 7H), 2.75 (bs, 4H), 2.66 (bs, 4H), 2.36 (bs, 3H), 2.29-2.27 (m, 2H), 1.94 (bs, 2H). ¹³C NMR (150 MHz, CDCl₃) δ ppm 175.2, 166.1, 144.2, 141.4, 133.2, 127.3, 114.0, 114.0, 56.3, 53.7, 52.5, 46.2, 45.6, 34.8, 33.7, 26.6, 24.1. LCMS m/z calcd for (M+H)⁺ 433.5, found 433.4.

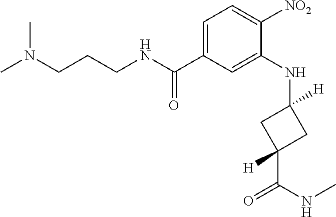

### N-(3-(dimethylamino)propyl)-3-(((1r,3r)-3-(methylcarbamoyl)cyclobutyl)amino)-4-nitrobenzamide

Molecular Formula: C₁₈H₂₇N₅O₄; yield 100%, yellow liquid. ¹H NMR (600 MHz, CDCl₃) δ ppm 8.69 (bs, 1H), 8.17 (d, J=8.8 Hz, 1H), 8.12 (d, J=5.3 Hz, 1H), 7.20 (d, J=1.2 Hz, 1H), 6.96 (dd, J=8.9, 1.5 Hz, 1H), 5.75 (bs, 1H), 4.42 (dd, J=13.4, 6.8 Hz, 1H), 3.54 (dd, J=11.4, 5.5 Hz, 2H), 3.03 (dd, J=9.1, 4.3 Hz, 3H), 2.84 (d, J=4.8 Hz, 3H), 2.60 (t. J=6.0 Hz, 2H), 2.38 (bs, 6H), 2.27-2.21 (m, 2H), 1.88-1.81 (m, 2H). ¹³C NMR (150 MHz, CDCl₃) δ ppm 175.2, 165.7, 144.1, 141.6, 133.1, 127.3, 113.8, 113.6, 58.7, 46.2, 45.1, 40.1, 34.9, 33.6, 26.6, 24.9. LCMS m/z calcd for (M+H)⁺ 378.4, found 378.4.

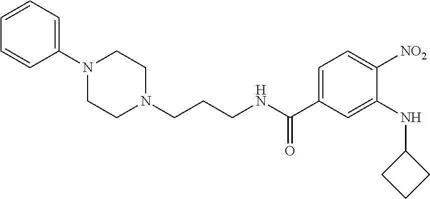 [SMILES: O=C(NCCCN1CCN(c2ccccc2)CC1)c1ccc([N+](=O)[O-])c(NC2CCC2)c1]

### 3-(cyclobutylamino)-4-nitro-N-(3-(4-phenylpiperazin-1-yl)propyl)benzamide

Molecular Formula: C₂₄H₃₁N₅O₃; yield 100%, yellow solid. ¹H NMR (600 MHz, CD₃OD) δ ppm 8.15 (d, J=8.8 Hz, 1H), 8.06 (d, J=5.4 Hz, 1H), 7.27 (d, J=1.5 Hz, 1H), 7.22 (dd, J=8.5, 7.5 Hz, 2H), 7.00 (dd, J=8.8, 1.7 Hz, 1H), 6.94 (d, J=8.1 Hz, 2H), 6.83 (t, J=7.3 Hz, 1H), 4.19 (dd, J=13.4, 7.4 Hz, 1H), 3.46 (t, J=6.9 Hz, 2H), 3.19-3.15 (m, 4H), 2.68-2.62 (m, 4H), 2.58-2.50 (m, 4H), 2.08-1.98 (m, 2H), 1.93-1.84 (m, 4H). ¹³C NMR (150 MHz, CD₃OD) δ ppm 168.5, 152.6, 145.1, 142.6, 134.1, 130.0, 128.0, 121.1, 117.4, 115.2, 114.3, 57.4, 54.3, 50.3, 39.8, 38.9, 31.7, 27.1, 16.2. LCMS m/z calcd for (M+H)⁺ 438.5, found 438.4.

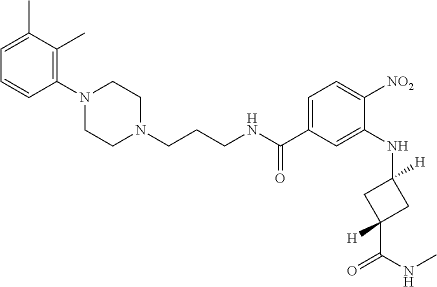 [SMILES: CNC(=O)[C@H]1C[C@H](Nc2cc(C(=O)NCCCN3CCN(c4cccc(C)c4C)CC3)ccc2[N+](=O)[O-])C1]

### N-(3-(4-(2,3-dimethylphenyl)piperazin-1-yl)propyl)-3-(((1r,3r)-3-(methylcarbamoyl)cyclobutyl)amino)-4-nitrobenzamide

Molecular Formula: C₂₈H₃₈N₆O₄; yield 79%, yellow solid. ¹H NMR (600 MHz, CD₃OD) δ ppm 8.20 (d, J=8.8 Hz, 1H), 8.10 (d, J=5.5 Hz, 1H), 7.89 (bs, 1H), 7.23 (d, J=1.3 Hz, 1H), 7.07 (dd, J=8.8, 1.6 Hz, 1H), 7.03 (t, J=7.7 Hz, 1H), 6.91 (t, J=7.0 Hz, 2H), 4.48-4.38 (m, 1H), 3.50 (t, J=6.7 Hz, 2H), 3.35 (bs, 1H), 3.23-3.18 (m, 2H), 3.12 (dd, J=9.4, 4.7 Hz, 1H), 3.00 (bs, 5H), 2.93-2.86 (m, 2H), 2.75 (bs, 5H), 2.34-2.27 (m, 2H), 2.24 (d, J=14.9 Hz, 6H), 2.02-1.96 (m, 2H). ¹³C NMR (150 MHz, CD₃OD) δ ppm 178.1, 168.9, 151.9, 145.1, 142.4, 139.1, 134.6, 132.3, 128.1, 127.0, 126.7, 117.7, 115.0, 114.8, 79.46, 56.8, 54.4, 52.0, 47.9, 47.3, 39.1, 35.4, 34.2, 26.5, 26.4, 20.6, 14.0. LCMS m/z calcd for (M+H)⁺ 523.6, found 523.5.

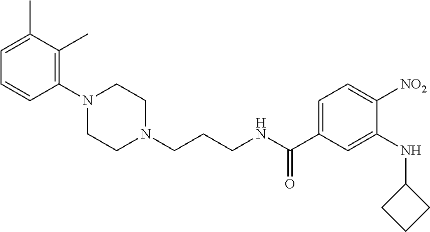 [SMILES: Cc1cccc(N2CCN(CCCNC(=O)c3ccc([N+](=O)[O-])c(NC4CCC4)c3)CC2)c1C]

### 3-(cyclobutylamino)-N-(3-(4-(2,3-dimethylphenyl)piperazin-1-yl)propyl)-4-nitro benzamide

Molecular Formula: C₂₆H₃₅N₅O₃; yield 59%, yellow solid. ¹H NMR (600 MHz, CD₃OD) δ ppm 8.19 (d, J=8.8 Hz, 1H), 7.89 (bs, 1H), 7.29 (d, J=1.7 Hz, 1H), 7.05-6.99 (m, 2H), 6.87 (t, J=7.7 Hz, 2H), 4.27-4.16 (m, 1H), 3.47 (t, J=6.8 Hz, 2H), 2.90 (bs, 4H), 2.73 (bs, 4H), 2.63-2.59 (m, 2H), 2.58-2.53 (m, 2H), 2.24 (s, 3H), 2.21 (s, 3H), 2.09-2.01 (m, 2H), 1.94-1.87 (m, 4H). ¹³C NMR (150 MHz, CD₃OD) δ ppm 168.7, 152.5, 145.2, 142.6, 138.9, 134.1, 132.2, 128.0, 126.9, 126.7, 117.6, 115.2, 114.3, 57.4, 54.8, 52.9, 39.8, 31.7, 26.9, 20.6, 16.2, 14.0. LCMS m/z calcd for (M+H)⁺ 466.6, found 466.4.

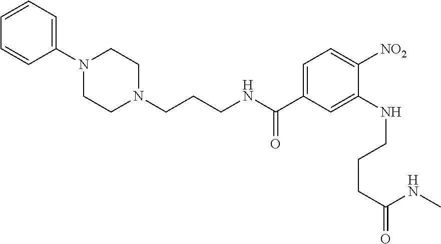 [SMILES: CNC(=O)CCCNc1cc(C(=O)NCCCN2CCN(c3ccccc3)CC2)ccc1[N+](=O)[O-]]

### 3-((4-(methylamino)-4-oxobutyl)amino)-4-nitro-N-(3-(4-phenylpiperazin-1-yl)propyl)benzamide

Molecular Formula: C₂₅H₃₄N₆O₄; yield 100%, yellow solid. ¹H NMR (600 MHz, CD₃OD) δ ppm 8.18 (d, J=8.7 Hz, 1H), 7.91 (bs, 1H), 7.48 (s, 1H), 7.24 (t, J=7.3 Hz, 2H), 7.06 (d, J=8.5 Hz, 1H), 6.97 (d, J=7.8 Hz, 2H), 6.87 (d, J=7.0 Hz, 1H), 3.51 (t, J=6.2 Hz, 2H), 3.46 (t, J=6.7 Hz, 2H), 3.25 (bs, 4H), 2.82 (bs, 4H), 2.73 (s, 3H), 2.69 (bs, 2H), 2.35 (t, J=6.8 Hz, 2H), 2.08-2.00 (m, 2H), 1.97 (d, J=6.5 Hz, 2H). ¹³C NMR (150 MHz, CD₃OD) δ ppm 175.7, 168.6, 152.4, 146.3, 142.4, 134.2, 130.1, 128.0, 121.3, 117.5, 114.5, 114.3, 57.0, 54.0, 49.9, 43.3, 39.45 34, 26.8, 26.4, 25.9. LCMS m/z calcd for (M+H)⁺ 483.6, found 483.5.

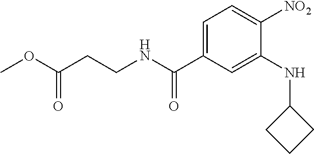 [SMILES: COC(=O)CCNC(=O)c1ccc([N+](=O)[O-])c(NC2CCC2)c1]

### Methyl 3-(3-(cyclobutylamino)-4-nitrobenzamido) propanoate

Molecular Formula: C₁₅H₁₉N₃O₅; yield 67%, yellow solid. ¹H NMR (600 MHz, CDCl₃) δ ppm 8.18 (d, J=8.8 Hz, 1H), 8.11 (bs, 1H), 7.21 (d, J=1.3 Hz, 1H), 6.92 (s, 1H), 6.83 (dd, J=8.8, 1.6 Hz, 1H), 4.17-4.09 (m, 1H), 3.73-3.70 (m, 5H), 2.66 (t, J=5.9 Hz, 2H), 2.58-2.51 (m, 2H), 2.05-2.03 (m, 2H), 1.90-1.89 (m, 2H). ¹³C NMR (150 MHz, CDCl₃) δ ppm 173.4, 166.0, 144.2, 141.1, 133.0, 127.5, 114.4, 112.5, 52.1, 48.1, 38.9, 35.6, 33.6, 31.0, 15.6. LCMS m/z calcd for (M+H)⁺ 322.4, found 322.4.

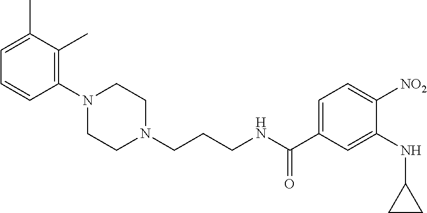 [SMILES: Cc1cccc(N2CCN(CCCNC(=O)c3ccc([N+](=O)[O-])c(NC4CC4)c3)CC2)c1C]

### 3-(cyclopropylamino)-N-(3-(4-(2,3-dimethylphenyl)piperazin-1-yl)propyl)-4-nitro benzamide

Molecular Formula: C₂₅H₃₃N₅O₃; yield 100%, yellow solid. ¹H NMR (600 MHz, CDCl₃) δ ppm 8.61 (bs, 1H), 8.19 (d, J=8.8 Hz, 1H), 8.10 (s, 1H), 7.90 (d, J=1.2 Hz, 1H), 7.11 (t, J=7.7 Hz, 1H), 7.04 (d, J=8.5 Hz, 1H), 6.93 (d, J=7.4 Hz, 1H), 6.84 (d, J=7.9 Hz, 1H), 3.63 (dd, J=11.2, 5.4 Hz, 2H), 2.94 (bs, 4H), 2.80 (s, 1H), 2.78-2.65 (m, 4H), 2.26 (s, 3H), 2.20 (s, 3H), 1.91 (bs, 2H), 1.25 (bs, 2H), 0.97 (d, J=5.4 Hz, 2H), 0.66 (dd, J=3.5, 1.7 Hz, 2H). ¹³C NMR (150 MHz, CDCl₃) δ ppm 166.3, 146.4, 138.3, 133.2, 131.4, 127.0, 126.3, 125.8, 116.8, 115.6, 113.6, 53.9, 51.8, 38.7, 29.8, 24.8, 24.0, 20.7, 13.9, 8.1. LCMS m/z calcd for (M+H)⁺ 452.6, found 452.5.

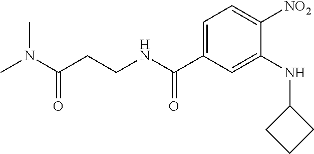 [SMILES: CN(C)C(=O)CCNC(=O)c1ccc([N+](=O)[O-])c(NC2CCC2)c1]

### 3-(cyclobutylamino)-N-(3-(dimethylamino)propyl)-4-nitrobenzamide

Molecular Formula: C₁₆H₂₄N₄O₃; yield 90%; ¹H NMR (600 MHz, DMSO-d₆) δ ppm 8.81 (t, J=5.5 Hz, 1H), 8.15 (d, J=8.9 Hz, 1H), 8.03 (d, J=6.1 Hz, 1H), 7.27 (s, 1H), 7.09 (dd, J=8.9, 1.4 Hz, 1H), 4.20 (dd, J=14.6, 7.3 Hz, 1H), 3.33-3.31 (m, 2H), 3.07-3.01 (m, 2H), 2.75 (s, 6H), 2.45 (d, J=8.1 Hz, 2H), 2.03 (dd, J=20.4, 9.4 Hz, 2H), 1.87 (dd, J=15.1, 7.3 Hz, 2H), 1.82-1.79 (m, 2H).). ¹³C NMR (150 MHz, CDCl₃) δ ppm 165.9, 144.3, 127.4, 114.5, 48.1, 44.5, 31.1, 24.4, 15.6. LCMS m/z calcd for (M+H)⁺ 321.3, found 321.3.

The following compounds were prepared according step 3 of the general procedure described herein:

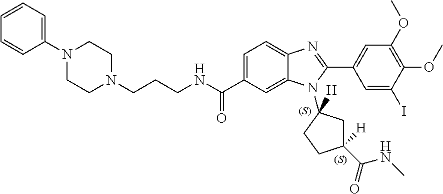

### 2-(3-iodo-4,5-dimethoxyphenyl)-1-((1S,3S)-3-(methylcarbamoyl)cyclopentyl)-N-(3-(4-phenylpiperazin-1-yl)propyl)-1H-benzo[d]imidazole-6-carboxamide

Molecular Formula: C₃₆H₄₃IN₆O₄; yield 51%, off-white solid. ¹H NMR (600 MHz, CDCl₃) δ ppm 8.41 (s, 1H), 8.18 (s, 1H), 7.69 (d, J=8.4 Hz, 1H), 7.63 (t, J=5.9 Hz, 2H), 7.30-7.26 (m, 3H), 7.18 (d, J=1.7 Hz, 1H), 6.91 (dd, J=15.8, 7.7 Hz, 3H), 6.07 (s, 1H), 5.26-5.19 (m, 1H), 3.93 (s, 6H), 3.68-3.65 (m, 2H), 3.25 (d, J=4.4 Hz, 4H), 3.21 (bs, 1H), 2.81 (d, J=4.8 Hz, 3H), 2.74 (bs, 2H), 2.69 (bs, 2H), 2.49-2.47 (m, 3H), 2.28-2.19 (m, 2H), 2.00-1.94 (m, 2H), 1.91 (dd, J=11.2, 5.8 Hz, 3H). ¹³C NMR (150 MHz, CDCl₃) δ ppm 175.4, 168.0, 156.7, 153.6, 146.3, 139.9, 133.1, 129.4, 128.1, 125.4, 121.0, 120.0, 116.7, 112.0, 106.9, 61.1, 57.1, 56.4, 53.2, 48.8, 45.0, 33.9, 31.9, 30.4, 26.4, 24.1. HRMS (HESI-TOF) m/z calcd for (M+H)⁺751.2469, found 751.2462.

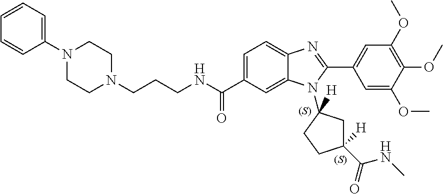

### 1-((1S,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide

Molecular Formula: C₃₇H₄₆N₆O₅; yield 60%, white solid. ¹H NMR (600 MHz, CDCl₃) δ ppm 8.40 (s, 1H), 8.19 (s, 1H), 7.71 (d, J=8.4 Hz, 1H), 7.63 (d, J=7.9 Hz, 1H), 7.28 (d, J=8.5 Hz, 2H), 6.91 (dd, J=18.0, 7.7 Hz, 3H), 6.87 (s, 2H), 6.04 (bs, 1H), 5.35-5.27 (m, 1H), 3.93 (d, J=8.1 Hz, 9H), 3.70-3.62 (m, 2H), 3.27 (bs, 4H), 2.81 (d, J=4.8 Hz, 3H), 2.75 (bs, 3H), 2.70 (bs, 2H), 2.59-2.42 (m, 3H), 2.32-2.16 (m, 2H), 2.01-1.85 (m, 4H). ¹³C NMR (150 MHz, CDCl₃) δ ppm 175.4, 168.0, 156.7, 153.6, 146.3, 139.9, 133.1, 129.4, 128.1, 125.4, 121.0, 120.0, 116.7, 112.0, 106.9, 61.1, 57.1, 56.4, 53.2, 48.8, 45.0, 33.9, 31.9, 30.4, 26.4, 24.1. HRMS (HESI-TOF) m/z calcd for (M+H)⁺ 655.3608, found 655.3591.

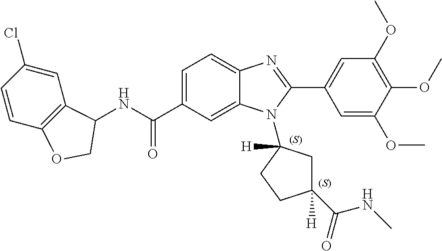

### N-(5-chloro-2,3-dihydrobenzofuran-3-yl)-1-((1S,3S)-3-(methylcarbamoyl)cyclo pentyl)-2-(3,4,5-trimethoxyphenyl)-1H-benzo[d]imidazole-6-carboxamide

Molecular Formula: C₃₂H₃₃ClN₄O₆; yield 77%, colorless liquid. ¹H NMR (600 MHz, CDCl₃) δ ppm 8.18 (s, 1H), 7.78 (d, J=8.4 Hz, 1H), 7.52 (d, J=8.5 Hz, 1H), 7.39 (t, J=2.4 Hz, 1H), 7.23 (d, J=8.5 Hz, 1H), 6.89 (s, 2H), 6.82 (d, J=8.6 Hz, 1H), 6.80 (d, J=7.3 Hz, 1H), 5.98 (d, J=3.3 Hz, 1H), 5.85-5.83 (m, 1H), 5.35 (t, J=9.0 Hz, 1H), 4.86 (dd, J=9.7, 8.5 Hz, 1H), 4.53-4.51 (m, 1H), 3.94 (d, J=5.9 Hz, 9H), 3.21-3.13 (m, 1H), 2.82 (d, J=4.8 Hz, 3H), 2.58-2.50 (m, 2H), 2.50-2.41 (m, 1H), 2.33-2.19 (m, 2H), 2.01-1.91 (m, 1H). ¹³C NMR (150 MHz, CDCl₃) δ ppm 175.2, 167.5, 159.3, 157.1, 153.7, 146.7, 140.057, 133.5, 130.6, 128.0, 127.030, 126.0, 125.6, 125.2, 120.1, 119.9, 112.8, 111.7, 106.9, 78.6, 78.5, 61.1, 57.2, 56.5, 52.4, 45.0, 33.9, 33.9, 31.7, 30.3, 26.6. HRMS (HESI-TOF) m/z calcd for (M+H)⁺ 605.2167, found 605.2156.

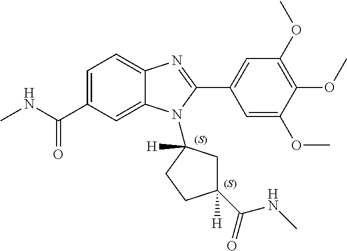

### N-methyl-1-((1S,3S)-3-(methylcarbamoyl)cyclopentyl)-2-(3,4,5-trimethoxyphenyl)-1H-benzo[d]imidazole-6-carboxamide

Molecular Formula: C₂₅H₃₀N₄O₅; yield 100%, white solid. ¹H NMR (600 MHz, CDCl₃) δ ppm 8.15 (s, 1H), 7.77 (d, J=8.4 Hz, 1H), 7.54 (dd, J=8.4, 1.1 Hz, 1H), 6.87 (s, 2H), 6.62 (d, J=4.6 Hz, 1H), 6.13 (d, J=4.6 Hz, 1H), 5.31 (dd, J=17.4, 9.0 Hz, 1H), 3.92 (d, J=7.2 Hz, 9H), 3.18-3.10 (m, 1H), 3.05 (d, J=4.8 Hz, 3H), 2.79 (d, J=4.8 Hz, 3H), 2.53-2.40 (m, 3H), 2.25-2.15 (m, 2H), 1.96-1.88 (m, 1H). ¹³C NMR (150 MHz, CDCl₃) δ ppm 175.3, 168.7, 156.8, 153.6, 140.0, 133.4, 128.3, 125.3, 120.0, 119.7, 112.5, 106.9, 61.1, 57.2, 56.4, 44.9, 33.8, 31.7, 30.3, 27.2, 26.5. HRMS (HESI-TOF) m/z calcd for (M+H)⁺ 467.2294, found 467.2287.

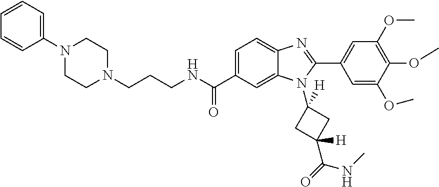 [SMILES: CNC(=O)[C@H]1C[C@H](n2c(-c3cc(OC)c(OC)c(OC)c3)nc3ccc(C(=O)NCCCN4CCN(c5ccccc5)CC4)cc32)C1]

### 1-((1r,3r)-3-(methylcarbamoyl)cyclobutyl)-N-(3-(4-phenylpiperazin-1-yl)propyl)-2-(3,4,5-trimethoxyphenyl)-1H-benzo[d]imidazole-6-carboxamide

Molecular Formula: C₃₆H₄₄N₆O₅; yield 70%, white solid. ¹H NMR (600 MHz, CDCl₃) δ ppm 8.39 (s, 1H), 8.29 (s, 1H), 7.71 (d, J=8.4 Hz, 1H), 7.66 (d, J=8.3 Hz, 1H), 7.28 (d, J=8.5 Hz, 2H), 6.91 (dd, J=11.0, 7.8 Hz, 3H), 6.84 (s, 2H), 5.69-5.59 (m, 2H), 3.93 (s, 9H), 3.67 (dd, J=11.2, 5.6 Hz, 2H), 3.28-3.24 (m, 4H), 3.18 (dd, J=19.0, 8.3 Hz, 3H), 2.84 (d, J=4.8 Hz, 3H), 2.76 (dd, J=6.6, 2.4 Hz, 5H), 2.73-2.68 (m, 2H), 1.95-1.90 (m, 2H). ¹³C NMR (150 MHz, CDCl₃) & 175.2, 167.8, 156.4, 153.6, 146.0, 140.0, 134.5, 129.4, 125.3, 120.5, 119.9, 116.5, 112.3, 106.9, 61.1, 56.5, 53.6, 49.6, 49.5, 33.6, 32.3, 29.8, 26.7, 24.6. HRMS (HESI-TOF) m/z calcd for (M+H)⁺ 641.7930, found 641.7929.

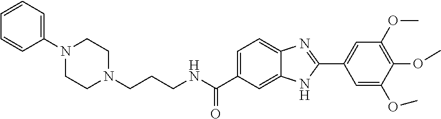 [SMILES: COc1cc(-c2nc3ccc(C(=O)NCCCN4CCN(c5ccccc5)CC4)cc3[nH]2)cc(OC)c1OC]

### N-(3-(4-phenylpiperazin-1-yl)propyl)-2-(3,4,5-trimethoxyphenyl)-1H-benzo[d]imidazole-6-carboxamide

Molecular Formula: C₃₀H₃₅N₅O₄; yield 100%, white solid. ¹H NMR (600 MHz, CDCl₃) δ ppm 8.34 (bs, 1H), 8.09 (bs, 1H), 7.55 (d, J=7.9 Hz, 1H), 7.37 (s, 2H), 7.21 (t, J=7.8 Hz, 2H), 6.89-6.81 (m, 3H), 3.84 (s, 3H), 3.69 (s, 6H), 3.53 (dd, J=10.9, 5.5 Hz, 2H), 3.17 (s, 4H), 2.65 (s, 4H), 2.58 (t, J=5.9 Hz, 2H), 1.83-1.76 (m, 2H). ¹³C NMR (150 MHz, CDCl₃) δ ppm 168.7, 153.7, 140.1, 129.3, 125.0, 120.3, 116.4, 104.5, 61.0, 57.8, 56.2, 53.4, 49.3, 40.7, 29.8, 24.7. HRMS (HESI-TOF) m/z calcd for (M+H)⁺ 530.2767, found 530.2764.

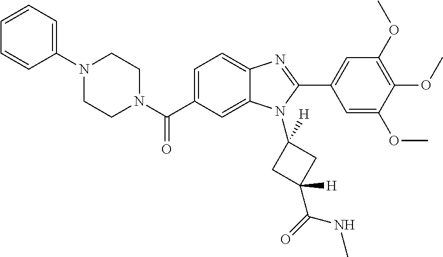 [SMILES: CNC(=O)[C@H]1C[C@H](n2c(-c3cc(OC)c(OC)c(OC)c3)nc3ccc(C(=O)N4CCN(c5ccccc5)CC4)cc32)C1]

### (1r,3r)-N-methyl-3-(6-(4-phenylpiperazine-1-carbonyl)-2-(3,4,5-trimethoxyphenyl)-1H-benzo[d]imidazol-1-yl)cyclobutane-1-carboxamide

Molecular Formula: C₃₃H₃₇N₅O₅; yield 77%, white solid. ¹H NMR (600 MHz, CDCl₃) δ ppm 7.99 (d, J=7.0 Hz, 1H), 7.88 (s, 1H), 7.45 (d, J=8.1 Hz, 1H), 7.30 (t, J=7.9 Hz, 2H), 6.99 (s, 4H), 6.95 (t, J=7.3 Hz, 1H), 5.78 (d, J=15.6 Hz, 2H), 3.97 (s, 7H), 3.94 (s, 4H), 3.25 (s, 4H), 3.07 (d, J=9.9 Hz, 4H), 2.81 (dd, J=22.6, 6.9 Hz, 6H), 2.61 (s, 2H). ¹³C NMR (150 MHz, CDCl₃) δ ppm 174.6, 153.8, 129.5, 117.1, 112.4, 107.4, 61.2, 56.7, 50.4, 41.2, 33.7, 32.7, 29.8, 26.7, 14.2. HRMS (HESI-TOF) m/z calcd for (M+H)⁺ 584.2873, found 584.2868.

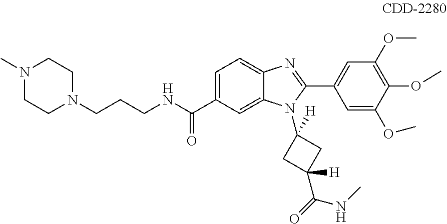

### 1-((1r,3r)-3-(methylcarbamoyl)cyclobutyl)-N-(3-(4-methylpiperazin-1-yl)propyl)-2-(3,4,5-trimethoxyphenyl)-1H-benzo[d]imidazole-6-carboxamide

Molecular Formula: C₃₁H₄₂N₆O₅; yield 63%, colorless liquid. ¹H NMR (600 MHz, CD₃OD) δ ppm 8.36 (s, 1H), 7.81 (dd, J=8.5, 1.3 Hz, 1H), 7.75 (d, J=8.5 Hz, 1H), 6.98 (s, 2H), 5.67 (t, J=8.8 Hz, 1H), 3.95 (s, 6H), 3.87 (s, 3H), 3.52 (t, J=6.9 Hz, 2H), 3.21-3.18 (m, 3H), 2.75 (bs, 5H), 2.68 (bs, 4H), 2.66 (s, 2H), 2.62 (t, J=7.2 Hz, 3H), 2.48 (s, 3H), 1.95-1.88 (m, 2H). ¹³C NMR (150 MHz, CD₃OD) δ ppm 177.9, 170.3, 154.9, 146.2, 141.3, 135.2, 130.3, 126.2, 122.7, 120.0, 113.2, 108.3, 61.2, 57.0, 56.8, 55.2, 53.0, 51.2, 45.2, 40.4, 39.4, 34.3, 33.3, 27.3, 26.5. HRMS (HESI-TOF) m/z calcd for (M+H)⁺ 579.3295, found 579.3290.

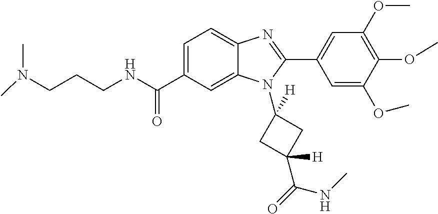

### N-(3-(dimethylamino)propyl)-1-((1r,3r)-3-(methylcarbamoyl)cyclobutyl)-2-(3,4,5-trimethoxyphenyl)-1H-benzo[d]imidazole-6-carboxamide

Molecular Formula: C₂₈H₃₇N₅O₅; yield 60%, %, colorless liquid. ¹H NMR (600 MHz, CDCl₃) δ ppm 8.63 (s, 1H), 8.41 (s, 1H), 7.81 (d, J=8.4 Hz, 1H), 7.72 (d, J=8.1 Hz, 1H), 6.85 (s, 2H), 5.98 (s, 1H), 5.64-5.56 (m, 1H), 3.93 (d, J=2.3 Hz, 9H), 3.68-3.62 (m, 2H), 3.47 (d, J=8.8 Hz, 1H), 3.25-3.17 (m, 2H), 2.86-2.76 (m, 8H), 2.53 (s, 6H), 1.99 (s, 2H). ¹³C NMR (150 MHz, CDCl₃) δ ppm 175.5, 168.1, 156.4, 153.5, 146.4, 139.9, 127.8, 125.4, 120.3, 110.6, 106.9, 61.1, 56.4, 49.3, 43.4, 33.6, 31.6, 26.5, 24.2. HRMS (HESI-TOF) m/z calcd for (M+H)⁺ 524.2873, found 524.2866.

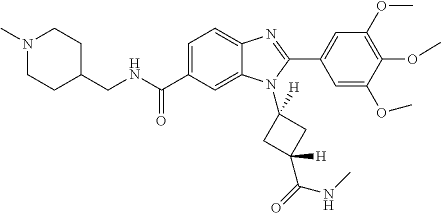

### 1-((1r,3r)-3-(methylcarbamoyl)cyclobutyl)-N-((1-methylpiperidin-4-yl)methyl)-2-(3,4,5-trimethoxyphenyl)-1H-benzo[d]imidazole-6-carboxamide

Molecular Formula: C₃₀H₃₉N₅O₅; yield 38%, colorless liquid. ¹H NMR (600 MHz, CDCl₃) δ ppm 8.31 (s, 1H), 7.75 (s, 2H), 7.60 (s, 1H), 6.83 (s, 2H), 6.35 (s, 1H), 5.59 (m, 1H), 3.91 (s, 9H), 3.43 (s, 2H), 3.26 (s, 3H), 3.17-3.06 (m, 2H), 2.80 (d, J=3.6 Hz, 3H), 2.72 (s, 2H), 2.60 (bs, 7H), 2.49 (d, J=7.6 Hz, 2H), 1.93 (bs, 3H), 1.77 (bs, 2H). ¹³C NMR (150 MHz, CDCl₃) δ ppm 175.2, 168.4, 156.4, 153.5, 146.1, 139.9, 134.3, 128.4, 125.2, 121.1, 119.9, 111.8, 106.8, 61.0, 56.4, 54.9, 49.5, 44.7, 41.1, 34.2, 33.3, 32.2, 28.2, 26.5. HRMS (HESI-TOF) m/z calcd for (M+H)⁺550.3029, found 550.3015.

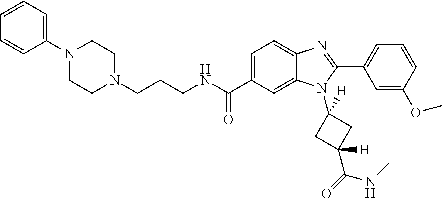 [SMILES: CNC(=O)[C@H]1C[C@H](n2c(-c3cccc(OC)c3)nc3ccc(C(=O)NCCCN4CCN(c5ccccc5)CC4)cc32)C1]

### 2-(3-methoxyphenyl)-1-((1r,3r)-3-(methylcarbamoyl)cyclobutyl)-N-(3-(4-phenyl piperazin-1-yl)propyl)-1H-benzo[d]imidazole-6-carboxamide

Molecular Formula: C₃₄H₄₀N₆O₃; yield 76%, off-white solid. ¹H NMR (600 MHz, CDCl₃) δ ppm 8.38 (s, 1H), 7.73 (s, 1H), 7.41 (t, J=7.9 Hz, 1H), 7.28-7.26 (m, 3H), 7.17 (s, 1H), 7.13 (d, J=7.6 Hz, 1H), 7.04 (dd, J=8.3, 2.2 Hz, 1H), 6.90 (t, J=7.7 Hz, 3H), 5.85 (s, 1H), 5.52 (m, 1H), 3.86 (s, 3H), 3.68 (dd, J=11.1, 5.5 Hz, 2H), 3.31 (bs, 5H), 3.15-3.08 (m, 2H), 2.92-2.73 (m, 10H), 1.97 (s, 2H). ¹³C NMR (150 MHz, CDCl₃) δ ppm 175.2, 167.8, 159.9, 156.3, 146.1, 134.3, 131.4, 129.9, 129.4, 128.6, 121.8, 120.7, 120.2, 120.0, 116.6, 114.6, 111.9, 55.5, 53.3, 49.4, 33.5, 32.0, 29.8, 26.6, 24.3. HRMS (HESI-TOF) m/z calcd for (M+H)⁺ 581.3240, found 581.3226.

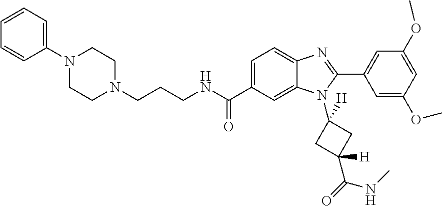 [SMILES: CNC(=O)[C@H]1C[C@H](n2c(-c3cc(OC)cc(OC)c3)nc3ccc(C(=O)NCCCN4CCN(c5ccccc5)CC4)cc32)C1]

### 2-(3,5-dimethoxyphenyl)-1-((1r,3r)-3-(methylcarbamoyl)cyclobutyl)-N-(3-(4-phenyl piperazin-1-yl)propyl)-1H-benzo[d]imidazole-6-carboxamide

Molecular Formula: C₃₅H₄₂N₆O₄; yield 57%, off-white solid. ¹H NMR (600 MHz, CDCl₃) δ ppm 8.39 (s, 1H), 7.74 (s, 1H), 7.28 (d, J=7.6 Hz, 3H), 6.93-6.88 (m, 3H), 6.72 (d, J=2.2 Hz, 2H), 6.59 (t, J=2.2 Hz, 1H), 5.84 (s, 1H), 5.53 (dd, J=17.2, 8.6 Hz, 1H), 3.84 (s, 6H), 3.68 (dd, J=11.1, 5.4 Hz, 2H), 3.32 (s, 4H), 3.18-3.09 (m, 2H), 2.88-2.74 (m, 10H), 1.98 (s, 2H). ¹³C NMR (150 MHz, CDCl₃) δ ppm 175.2, 167.8, 161.1, 156.4, 146.1, 134.3, 131.9, 129.4, 128.6, 120.1, 116.7, 111.9, 107.5, 55.7, 53.3, 49.4, 49.0, 33.6, 32.0, 29.8, 26.6, 24.4. HRMS (HESI-TOF) m/z calcd for (M+H)⁺ 611.3346, found 611.3342.

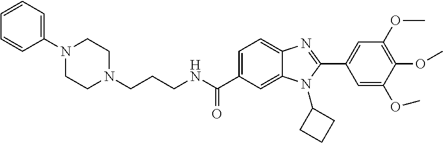 [SMILES: COc1cc(-c2nc3ccc(C(=O)NCCCN4CCN(c5ccccc5)CC4)cc3n2C2CCC2)cc(OC)c1OC]

### 1-cyclobutyl-N-(3-(4-phenylpiperazin-1-yl)propyl)-2-(3,4,5-trimethoxyphenyl)-1H-benzo[d]imidazole-6-carboxamide

Molecular Formula: C₃₄H₄₁N₅O₄; yield 99%, white solid. ¹H NMR (600 MHz, CDCl₃) δ ppm 8.44 (s, 1H), 8.37 (s, 1H), 7.73 (d, J=8.0 Hz, 2H), 7.25 (d, J=6.7 Hz, 4H), 6.92-6.87 (m, 3H), 6.83 (s, 2H), 5.11 (m, 1H), 3.91 (d, J=8.8 Hz, 9H), 3.68 (d, J=5.8 Hz, 2H), 3.35 (d, J=9.3 Hz, 4H), 3.07-2.78 (m, 8H), 2.42 (dd, J=18.1, 8.4 Hz, 2H), 2.07 (dd, J=26.7, 16.2 Hz, 3H), 1.89-1.77 (m, 1H). ¹³C NMR (150 MHz, CDCl₃) δ ppm 167.9, 156.0, 153.5, 145.9, 139.8, 134.9, 129.4, 126.0, 120.9, 119.8, 116.8, 112.6, 107.0, 61.1, 56.5, 53.1, 51.4, 48.7, 29.8, 29.7, 24.3, 15.0. HRMS (HESI-TOF) m/z calcd for (M+H)⁺ 584.3237, found 584.3217.

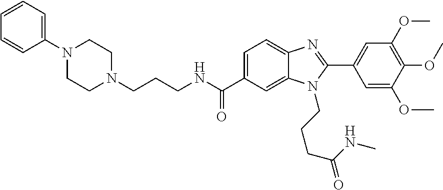 [SMILES: CNC(=O)CCCn1c(-c2cc(OC)c(OC)c(OC)c2)nc2ccc(C(=O)NCCCN3CCN(c4ccccc4)CC3)cc21]

### 1-(4-(methylamino)-4-oxobutyl)-N-(3-(4-phenylpiperazin-1-yl)propyl)-2-(3,4,5-trimethoxy phenyl)-1H-benzo[d]imidazole-6-carboxamide

Molecular Formula: C₃₅H₄₄N₆O₅; yield 36%, off-white solid. ¹H NMR (600 MHz, CDCl₃) δ ppm 8.63 (s, 1H), 8.30 (s, 1H), 7.93 (d, J=8.3 Hz, 1H), 7.79 (d, J=8.4 Hz, 1H), 7.29 (t, J=8.0 Hz, 3H), 6.96-6.93 (m, 3H), 6.89 (d, J=8.0 Hz, 2H), 4.34-4.30 (m, 2H), 3.93 (d, J=2.9 Hz, 9H), 3.70 (d, J=5.5 Hz, 2H), 3.50 (bs, 4H), 3.08 (bs, 4H), 2.70 (d, J=4.8 Hz, 3H), 2.36-2.17 (m, 8H). ¹³C NMR (150 MHz, CDCl₃) δ ppm 172.5, 168.4, 156.0, 153.7, 145.7, 140.0, 135.7, 129.7, 125.3, 119.8, 117.2, 109.9, 106.8, 61.1, 56.6, 52.6, 47.5, 44.9, 34.1, 29.8, 27.0, 26.3, 23.7. HRMS (HESI-TOF) m/z calcd for (M+H)⁺ 629.3451, found 629.3442.

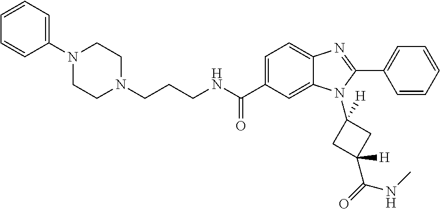 [SMILES: CNC(=O)[C@H]1C[C@H](n2c(-c3ccccc3)nc3ccc(C(=O)NCCCN4CCN(c5ccccc5)CC4)cc32)C1]

### 1-((1r,3r)-3-(methylcarbamoyl)cyclobutyl)-2-phenyl-N-(3-(4-phenylpiperazin-1-yl)propyl)-1H-benzo[d]imidazole-6-carboxamide

Molecular Formula: C₃₃H₃₈N₆O₂; yield 90%, off-white solid. ¹H NMR (600 MHz, CDCl₃) δ ppm 8.40 (d, J=16.6 Hz, 2H), 7.75 (s, 2H), 7.60 (dd, J=6.6, 2.9 Hz, 2H), 7.53-7.48 (m, 3H), 7.28 (d, J=7.9 Hz, 3H), 6.91 (dd, J=7.5, 5.4 Hz, 3H), 5.96 (s, 1H), 5.55-5.46 (m, 1H), 3.68 (dd, J=11.3, 5.5 Hz, 2H), 3.34 (d, J=2.6 Hz, 4H), 3.18-3.08 (m, 2H), 2.86 (d, J=21.5 Hz, 4H), 2.81 (t, J=5.7 Hz, 5H), 2.80-2.75 (m, 2H), 2.01 (s, 2H). ¹³C NMR (150 MHz, CDCl₃) δ ppm 175.3, 167.9, 156.5, 146.3, 134.3, 130.3, 130.3, 129.6, 129.5, 128.9, 128.5, 120.9, 120.1, 116.7, 111.8, 53.2, 49.4, 48.8, 33.6, 32.1, 29.8, 26.6, 24.3. HRMS (HESI-TOF) m/z calcd for (M+H)⁺ 551.3134, found 551.3138.

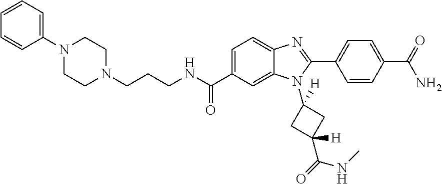 [SMILES: CNC(=O)[C@H]1C[C@H](n2c(-c3ccc(C(N)=O)cc3)nc3ccc(C(=O)NCCCN4CCN(c5ccccc5)CC4)cc32)C1]

### 2-(4-carbamoylphenyl)-1-((1r,3r)-3-(methylcarbamoyl)cyclobutyl)-N-(3-(4-phenyl piperazin-1-yl)propyl)-1H-benzo[d]imidazole-6-carboxamide

Molecular Formula: C₃₄H₃₉N₇O₃; yield 58%, white solid. ¹H NMR (600 MHz, CDCl₃) δ ppm 8.47 (s, 1H), 8.37 (s, 1H), 7.96 (d, J=8.3 Hz, 2H), 7.75 (t, J=8.3 Hz, 2H), 7.68 (d, J=8.3 Hz, 2H), 7.28 (d, J=8.3 Hz, 3H), 6.91 (t, J=7.1 Hz, 3H), 6.57 (s, 1H), 6.05 (bs, 1H), 5.80 (bs, 1H), 5.47-5.45 (m, 1H), 3.69 (dd, J=11.1, 5.4 Hz, 2H), 3.34 (d, J=5.9 Hz, 4H), 3.10 (dd, J=21.5, 10.5 Hz, 2H), 2.84 (t, J=16.7 Hz, 8H), 2.72 (bs, 3H), 2.01 (bs, 2H). ¹³C NMR (150 MHz, CDCl₃) δ ppm 175.3, 168.6, 167.8, 155.1, 146.1, 134.9, 133.7, 129.8, 129.5, 128.0, 120.3, 116.7, 53.3, 49.6, 48.8, 33.6, 32.3, 29.8, 26.6, 24.3. HRMS (HESI-TOF) m/z calcd for (M+H)⁺ 594.3193, found 594.3185.

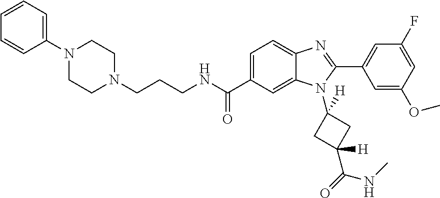 [SMILES: CNC(=O)[C@H]1C[C@H](n2c(-c3cc(F)cc(OC)c3)nc3ccc(C(=O)NCCCN4CCN(c5ccccc5)CC4)cc32)C1]

### 2-(3-fluoro-5-methoxyphenyl)-1-((1r,3r)-3-(methylcarbamoyl)cyclobutyl)-N-(3-(4-phenylpiperazin-1-yl)propyl)-1H-benzo[d]imidazole-6-carboxamide

Molecular Formula: C₃₄H₃₉FN₆₀₃; yield 65%, colorless liquid. ¹H NMR (600 MHz, CDCl₃) δ ppm 8.39 (d, J=13.3 Hz, 2H), 7.73 (s, 2H), 7.30-7.24 (m, 3H), 6.90 (dt, J=7.7, 4.7 Hz, 5H), 6.76 (dt, J=10.4, 2.2 Hz, 1H), 5.84 (s, 1H), 5.55-5.45 (m, 1H), 3.86 (s, 3H), 3.68 (dd, J=11.2, 5.5 Hz, 2H), 3.31 (bs, 4H), 3.17-3.09 (m, 2H), 2.89-2.74 (m, 10H), 1.98 (bs, 2H). 13C NMR (150 MHz, CDCl₃) δ ppm 175.1, 167.7, 164.3, 162.7, 161.3, 161.2, 155.1, 150.8, 146.0, 134.3, 132.5, 129.4, 129.0, 120.8, 120.2, 116.6, 112.0, 111.0, 109.1, 108.9, 104.0, 103.9, 56.0, 53.3, 49.4, 49.0, 33.6, 32.1, 29.8, 26.6, 24.4. HRMS (HESI-TOF) m/z calcd for (M+H)⁺ 599.3146, found 599.3137.

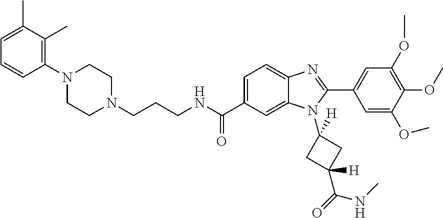 [SMILES: CNC(=O)[C@H]1C[C@H](n2c(-c3cc(OC)c(OC)c(OC)c3)nc3ccc(C(=O)NCCCN4CCN(c5cccc(C)c5C)CC4)cc32)C1]

### N-(3-(4-(2,3-dimethylphenyl)piperazin-1-yl)propyl)-1-((1r,3r)-3-(methylcarbamoyl)cyclobutyl)-2-(3,4,5-trimethoxyphenyl)-1H-benzo[d]imidazole-6-carboxamide

Molecular Formula: C₃₈H₄₈N₆O₅; yield 64%, white solid. ¹H NMR (600 MHz, CDCl₃) δ ppm 8.66 (s, 1H), 8.43 (s, 1H), 7.81 (s, 2H), 7.10 (t, J=7.7 Hz, 1H), 6.91 (dd, J=19.1, 7.6 Hz, 2H), 6.85 (s, 2H), 5.95 (s, 1H), 5.61 (m, 1H), 3.93 (s, 10H), 3.69 (d, J=4.4 Hz, 2H), 3.47 (bs, 3H), 3.36 (d, J=8.4 Hz, 1H), 3.21 (dd, J=20.5, 10.1 Hz, 2H), 3.04 (bs, 4H), 2.87-2.75 (m, 9H), 2.26 (s, 3H), 2.20 (s, 3H), 1.99 (bs, 2H). ¹³C NMR (150 MHz, CDCl₃) δ ppm 175.4, 168.0, 156.5, 153.6, 146.2, 139.9, 138.4, 134.2, 131.4, 128.4, 126.3, 125.4, 120.0, 116.9, 106.9, 61.1, 56.5, 53.7, 49.5, 33.6, 32.0, 29.8, 26.6, 24.0, 20.7, 13.9. HRMS (HESI-TOF) m/z calcd for (M+H)⁺ 669.3764, found 669.3743.

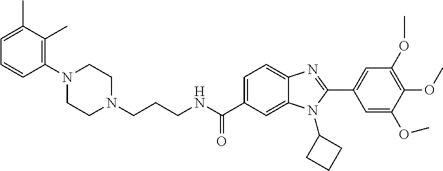 [SMILES: COc1cc(-c2nc3ccc(C(=O)NCCCN4CCN(c5cccc(C)c5C)CC4)cc3n2C2CCC2)cc(OC)c1OC]

### 1-cyclobutyl-N-(3-(4-(2,3-dimethylphenyl)piperazin-1-yl)propyl)-2-(3,4,5-trimethoxy phenyl)-1H-benzo[d]imidazole-6-carboxamide

Molecular Formula: C₃₆H₄₅N₅O₄; yield 69%, white solid. ¹H NMR (600 MHz, CDCl₃) δ ppm 8.67 (s, 1H), 8.50 (s, 1H), 8.06-7.83 (m, 1H), 7.82 (d, J=8.4 Hz, 1H), 7.09 (t, J=7.7 Hz, 1H), 6.94 (dd, J=11.1, 8.1 Hz, 2H), 6.86 (d, J=6.2 Hz, 2H), 5.18-5.08 (m, 1H), 3.93 (d, J=6.6 Hz, 9H), 3.73 (d, J=4.6 Hz, 2H), 3.17-2.87 (m, 8H), 2.47 (d, J=8.4 Hz, 2H), 2.26 (s, 3H), 2.19 (s, 3H), 2.13 (d, J=9.3 Hz, 1H), 1.92-1.82 (m, 1H), 1.60 (bs, 5H). ¹³C NMR (150 MHz, CD₃OD) δ ppm 154.9, 152.3, 146.1, 141.2, 139.0, 135.7, 132.3, 130.2, 127.0, 126.7, 126.4, 122.7, 119.9, 117.6, 113.5, 108.3, 79.5, 61.3, 56.9, 54.7, 52.8, 52.7, 39.7, 30.6, 27.0, 20.6, 15.7, 14.0. HRMS (HESI-TOF) m/z calcd for (M+H)⁺ 612.3550, found 612.3548. HRMS (HESI-TOF) m/z calcd for (M+H)⁺ 612.3550, found 612.3544.

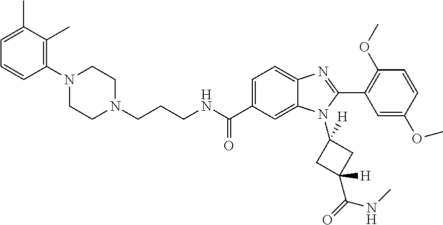 [SMILES: CNC(=O)[C@H]1C[C@H](n2c(-c3cc(OC)ccc3OC)nc3ccc(C(=O)NCCCN4CCN(c5cccc(C)c5C)CC4)cc32)C1]

### 2-(2,5-dimethoxyphenyl)-N-(3-(4-(2,3-dimethylphenyl)piperazin-1-yl)propyl)-1-((1r,3r)-3-(methylcarbamoyl)cyclobutyl)-1H-benzo[d]imidazole-6-carboxamide

Molecular Formula: C₃₇H₄₆N₆O₄; yield 60%, off-white solid. ¹H NMR (600 MHz, CDCl₃) δ ppm 8.61 (bs, 1H), 8.42 (s, 1H), 7.81 (t, J=7.5 Hz, 2H), 7.10 (dd, J=7.7, 5.6 Hz, 2H), 7.03 (dd, J=9.0, 3.1 Hz, 1H), 6.93 (dd, J=11.3, 7.1 Hz, 3H), 5.92 (bs, 1H), 5.12 (m, 1H), 3.80 (s, 3H), 3.74 (s, 3H), 3.69 (d, J=5.5 Hz, 2H), 3.35 (d, J=6.4 Hz, 1H), 3.13 (d, J=8.0 Hz, 2H), 3.05 (s, 4H), 2.81 (d, J=4.8 Hz, 8H), 2.26 (s, 3H), 2.20 (s, 3H), 1.99 (bs, 2H). ¹³C NMR (150 MHz, CDCl₃) δ ppm 175.6, 167.9, 154.1, 153.8, 151.7, 146.5, 138.3, 133.9, 131.4, 126.3, 125.8, 119.9, 117.9, 116.8, 116.8, 112.3, 56.1, 53.9, 49.7, 33.7, 31.9, 26.6, 24.2, 20.7, 13.9. HRMS (HESI-TOF) m/z calcd for (M+H)⁺ 639.3659, found 639.3656.

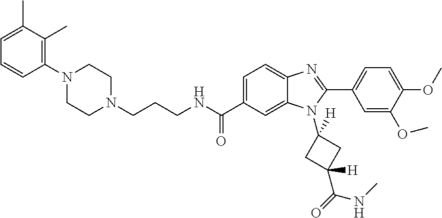 [SMILES: CNC(=O)[C@H]1C[C@H](n2c(-c3ccc(OC)c(OC)c3)nc3ccc(C(=O)NCCCN4CCN(c5cccc(C)c5C)CC4)cc32)C1]

### 2-(3,4-dimethoxyphenyl)-N-(3-(4-(2,3-dimethylphenyl)piperazin-1-yl)propyl)-1-((1r,3r)-3-(methylcarbamoyl)cyclobutyl)-1H-benzo[d]imidazole-6-carboxamide

Molecular Formula: C₃₇H₄₆N₆O₄; yield 64%, white solid. ¹H NMR (600 MHz, CDCl₃) δ ppm 8.64 (bs, 1H), 8.42 (s, 1H), 7.81 (d, J=7.1 Hz, 2H), 7.24 (d, J=1.7 Hz, 1H), 7.13 (dd, J=8.2, 1.7 Hz, 1H), 7.10 (t, J=7.8 Hz, 1H), 7.00 (d, J=8.3 Hz, 1H), 6.92 (dd, J=18.6, 7.7 Hz, 2H), 5.93 (bs, 1H), 5.59 (m, 1H), 3.96 (d, J=3.4 Hz, 6H), 3.70 (d, J=5.2 Hz, 2H), 3.35 (s, 1H), 3.19 (d, J=11.1 Hz, 2H), 3.06 (s, 4H), 2.90-2.74 (m, 8H), 2.26 (s, 3H), 2.20 (s, 3H), 2.01 (bs, 2H). ¹³C NMR (150 MHz, CDCl₃) δ ppm 175.4, 167.9, 156.6, 149.4, 146.3, 138.3, 134.3, 131.4, 126.3, 122.7, 122.4, 116.8, 112.7, 111.2, 56.2, 56.2, 53.8, 49.5, 33.6, 32.1, 29.8, 26.6, 24.1, 20.7, 13.9. HRMS (HESI-TOF) m/z calcd for (M+H)⁺ 639.3659, found 639.3660.

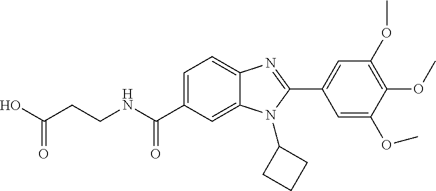 [SMILES: COc1cc(-c2nc3ccc(C(=O)NCCC(=O)O)cc3n2C2CCC2)cc(OC)c1OC]

### 3-(1-cyclobutyl-2-(3,4,5-trimethoxyphenyl)-1H-benzo[d]imidazole-6-carboxamido)propanoic acid

Molecular Formula: C₂₄H₂₇N₃O₆; yield 60%, white solid. ¹H NMR (600 MHz, CD₃OD) δ ppm 8.35 (bs, 1H), 7.78 (d, J=8.4 Hz, 1H), 7.72 (d, J=8.5 Hz, 1H), 6.96 (s, 2H), 5.24 (m, 1H), 3.92 (s, 6H), 3.86 (s, 3H), 3.69 (d, J=6.9 Hz, 2H), 2.90-2.82 (m, 2H), 2.70 (t, J=6.8 Hz, 2H), 2.50 (dd, J=17.8, 8.3 Hz, 2H), 2.06-2.00 (m, 1H), 1.93 (dd, J=19.2, 8.7 Hz, 1H). ¹³C NMR (150 MHz, CD₃OD) δ ppm 175.5, 170.4, 157.3, 154.8, 146.0, 141.2, 135.5, 130.2, 126.6, 122.7, 119.8, 113.5, 108.3, 61.3, 56.9, 52.8, 37.3, 34.8, 30.5, 15.7. HRMS (HESI-TOF) m/z calcd for (M+H)⁺ 454.1978, found 454.1970.

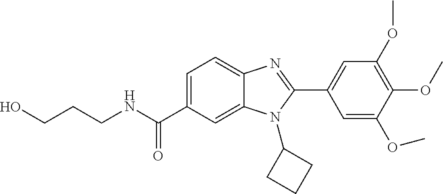 [SMILES: COc1cc(-c2nc3ccc(C(=O)NCCCO)cc3n2C2CCC2)cc(OC)c1OC]

### 1-cyclobutyl-N-(3-hydroxypropyl)-2-(3,4,5-trimethoxyphenyl)-1H-benzo[d]imidazole-6-carboxamide

Molecular Formula: C₂₄H₂₉N₃O₅; yield 80%, white solid. ¹H NMR (600 MHz, CDCl₃) δ ppm 8.36 (s, 1H), 7.80 (d, J=8.4 Hz, 1H), 7.59 (d, J=8.2 Hz, 1H), 7.07 (s, 1H), 6.88 (s, 2H), 5.23-5.15 (m, 1H), 3.96-3.91 (m, 9H), 3.79 (dd, J=11.9, 6.0 Hz, 2H), 3.74 (s, 3H), 3.00-2.85 (m, 2H), 2.72 (t, J=5.9 Hz, 2H), 2.47 (dd, J=18.2, 8.5 Hz, 2H), 2.07 (q, J=10.3 Hz, 1H), 1.96-1.85 (m, 1H). ¹³C NMR (150 MHz, CD₃OD) δ ppm 170.3, 156.9, 154.9, 145.0, 141.4, 135.4, 130.6, 126.0, 123.0, 119.4, 113.6, 108.3, 61.3, 60.8, 56.9, 52.9, 38.5, 33.3, 30.6, 15.7. HRMS (HESI-TOF) m/z calcd for (M+H)⁺440.2185, found 440.2184.

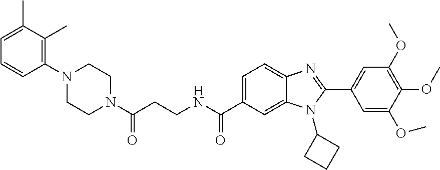 [SMILES: COc1cc(-c2nc3ccc(C(=O)NCCC(=O)N4CCN(c5cccc(C)c5C)CC4)cc3n2C2CCC2)cc(OC)c1OC]

### 1-cyclobutyl-N-(3-(4-(2,3-dimethylphenyl)piperazin-1-yl)-3-oxopropyl)-2-(3,4,5-trimethoxyphenyl)-1H-benzo[d]imidazole-6-carboxamide

Molecular Formula: C₃₆H₄₃N₅O₅; yield 80%, white solid. ¹H NMR (600 MHz, CDCl₃) δ ppm 8.37 (s, 1H), 7.82 (d, J=8.4 Hz, 1H), 7.65 (d, J=8.4 Hz, 1H), 7.46 (s, 1H), 7.06 (t, J=7.7 Hz, 1H), 6.95-6.89 (m, 3H), 6.84 (d, J=8.0 Hz, 1H), 5.22 (s, 1H), 3.94 (d, J=2.1 Hz, 9H), 3.89-3.82 (m, 2H), 3.63 (bs, 2H), 3.06 (d, J=25.4 Hz, 1H), 2.97-2.77 (m, 6H), 2.75 (t, J=5.4 Hz, 2H), 2.49 (d, J=9.0 Hz, 2H), 2.27 (s, 3H), 2.24 (s, 3H), 2.07 (q, J=10.3 Hz, 1H), 1.90 (dd, J=19.6, 9.3 Hz, 1H). ¹³C NMR (150 MHz, CDCl₃) δ ppm 170.6, 153.6, 150.9, 138.4, 131.5, 126.1, 125.8, 116.9, 107.3, 61.2, 56.6, 52.4, 52.2, 46.1, 42.3, 36.0, 33.0, 29.7, 20.8, 15.0, 14.0. HRMS (HESI-TOF) m/z calcd for (M+H)⁺ 626.3342, found 626.3327.

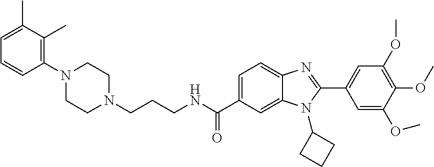 [SMILES: COc1cc(-c2nc3ccc(C(=O)NCCCN4CCN(c5cccc(C)c5C)CC4)cc3n2C2CCC2)cc(OC)c1OC]

### 1-cyclopropyl-N-(3-(4-(2,3-dimethylphenyl)piperazin-1-yl)propyl)-2-(3,4,5-trimethoxyphenyl)-1H-benzo[d]imidazole-6-carboxamide

Molecular Formula: C₃₅H₄₃N₅O₄; yield 38%, colorless liquid. ¹H NMR (600 MHz, CDCl₃) δ ppm 8.41 (s, 1H), 8.12 (d, J=9.3 Hz, 1H), 7.76 (d, J=8.1 Hz, 1H), 7.65 (s, 1H), 7.52 (s, 2H), 7.01 (d, J=7.3 Hz, 1H), 6.92 (d, J=7.3 Hz, 1H), 6.84 (d, J=7.9 Hz, 1H), 3.96 (d, J=10.6 Hz, 1H), 3.86 (d, J=13.9 Hz, 9H), 3.49 (s, 2H), 3.09 (s, 4H), 2.93 (s, 4H), 2.25 (d, J=1.6 Hz, 1H), 2.24 (s, 3H), 2.18 (d, J=5.9 Hz, 1H), 2.15 (s, 3H), 2.01 (bs, 2H), 1.25 (bs, 4H). HRMS (HESI-TOF) m/z calcd for (M+H)⁺ 598.3393, found 598.3394.

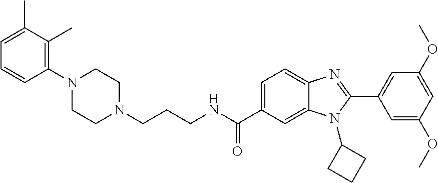 [SMILES: COc1cc(OC)cc(-c2nc3ccc(C(=O)NCCCN4CCN(c5cccc(C)c5C)CC4)cc3n2C2CCC2)c1]

### 1-cyclobutyl-2-(3,5-dimethoxyphenyl)-N-(3-(4-(2,3-dimethylphenyl)piperazin-1-yl)propyl)-1H-benzo[d]imidazole-6-carboxamide

Molecular Formula: C₃₅H₄₃N₅O₃; yield 80%, white solid. ¹H NMR (600 MHz, CDCl₃) δ ppm 8.54 (bs, 1H), 8.47 (s, 1H), 7.81 (d, J=8.3 Hz, 1H), 7.77 (s, 1H), 7.10 (t, J=7.7 Hz, 1H), 6.91 (dd, J=12.8, 7.7 Hz, 2H), 6.77 (d, J=2.2 Hz, 2H), 6.61 (t, J=2.2 Hz, 1H), 5.13 (m, 1H), 3.85 (s, 6H), 3.68 (d, J=5.5 Hz, 2H), 3.06-2.89 (m, 6H), 2.77 (s, 4H), 2.44 (d, J=9.8 Hz, 2H), 2.26 (s, 3H), 2.20 (s, 3H), 2.05 (d, J=10.7 Hz, 1H), 1.95 (s, 2H), 1.85 (dd, J=19.6, 8.9 Hz, 1H). ¹³C NMR (150 MHz, CDCl₃) δ ppm 161.0, 155.9, 138.2, 135.0, 132.5, 131.4, 126.3, 119.8, 116.9, 112.8, 107.7, 102.5, 55.7, 53.9, 51.3, 29.7, 20.7, 15.0, 14.0. HRMS (HESI-TOF) m/z calcd for (M+H)⁺ 582.3444, found 582.3433.

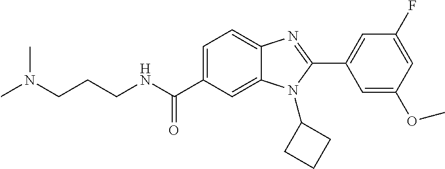

### 1-cyclobutyl-N-(3-(dimethylamino)propyl)-2-(3-fluoro-5-methoxyphenyl)-1H-benzo[d]imidazole-6-carboxamide

Molecular Formula: C₂₄H₂₉FN₄O₂; yield 56%, colorless liquid. ¹H NMR (600 MHz, CDCl₃) δ ppm 8.58 (s, 1H), 8.40 (s, 1H), 7.78 (d, J=8.4 Hz, 1H), 7.59 (d, J=8.2 Hz, 1H), 6.99 (s, 1H), 6.92 (d, J=8.0 Hz, 1H), 6.77 (d, J=10.5 Hz, 1H), 5.12-5.06 (m, J=8.8 Hz, 1H), 3.86 (s, 3H), 3.67-3.60 (m, 2H), 2.95-2.86 (m, 2H), 2.58 (t, J=5.8 Hz, 2H), 2.46 (dd, J=18.2, 8.4 Hz, 2H), 2.36 (s, 6H), 2.03 (d, J=10.5 Hz, 1H), 1.90-1.81 (m, 3H). ¹³C NMR (150 MHz, CDCl₃) δ ppm 167.5, 164.2, 162.6, 161.2, 161.2, 154.56, 145.65, 134.94, 133.13, 133.06, 129.39, 120.41, 119.97, 112.65, 111.21, 111.2, 109.1, 108.9, 103.7, 103.5, 59.2, 56.0, 51.3, 45.4, 40.6, 29.7, 25.3, 14.9. HRMS (HESI-TOF) m/z calcd for (M+H)⁺ 425.2352, found 425.2338.

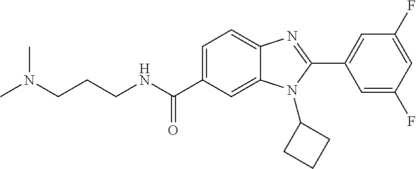 [SMILES: CN(C)CCCNC(=O)c1ccc2nc(-c3cc(F)cc(F)c3)n(C3CCC3)c2c1]

### 1-cyclobutyl-2-(3,5-difluorophenyl)-N-(3-(dimethylamino)propyl)-1H-benzo[d]imidazole-6-carboxamide

Molecular Formula: C₂₃H₂₆F₂N₄O; yield 87%, colorless liquid. ¹H NMR (600 MHz, CDCl₃) δ ppm 8.64 (s, 1H), 8.41 (s, 1H), 7.79 (d, J=8.4 Hz, 1H), 7.61 (d, J=8.2 Hz, 1H), 7.19 (d, J=5.3 Hz, 2H), 6.98 (t, J=8.7 Hz, 1H), 5.10-5.04 (m, 1H), 3.64 (dd, J=11.2, 5.5 Hz, 2H), 2.93-2.86 (m, 2H), 2.61 (t, J=5.7 Hz, 2H), 2.51-2.45 (m, 2H), 2.38 (s, 6H), 2.06-2.04 (m, 1H), 1.93-1.83 (m, 3H). ¹³C NMR (150 MHz, CDCl₃) δ ppm 167.4, 163.9, 163.8, 162.2, 162.2, 153.2, 145.6, 135.0, 133.9, 129.7, 113.0, 112.9, 112.8, 112.8, 105.7, 105.5, 59.2, 51.4, 45.3, 40.6, 29.8, 25.2, 15.0. HRMS (HESI-TOF) m/z calcd for (M+H)⁺ 413.2152, found 413.2147.

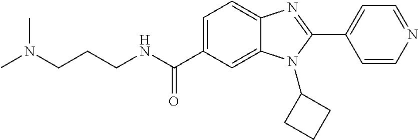 [SMILES: CN(C)CCCNC(=O)c1ccc2nc(-c3ccncc3)n(C3CCC3)c2c1]

### 1-cyclobutyl-N-(3-(dimethylamino)propyl)-2-(pyridin-4-yl)-1H-benzo[d]imidazole-6-carboxamide

Molecular Formula: C₂₂H₂₇FN₅O₂; yield 72%, white solid. ¹H NMR (600 MHz, CDCl₃) δ ppm 8.80 (d, J=4.8 Hz, 2H), 8.65 (s, 1H), 8.43 (s, 1H), 7.81 (d, J=8.4 Hz, 1H), 7.65 (s, 1H), 7.58 (d, J=5.3 Hz, 2H), 5.11-5.05 (m, 1H), 3.64 (d, J=5.2 Hz, 2H), 2.93-2.91 (m 2H), 2.64 (s, 2H), 2.49 (dd, J=18.0, 8.5 Hz, 2H), 2.40 (s, 6H), 2.07-2.05 (m, 1H), 1.92-1.84 (m, 3H). ¹³C NMR (150 MHz, CDCl₃) δ ppm 167.4, 153.0, 150.4, 145.79, 138.6, 135.1, 129.8, 123.8, 120.3, 112.8, 59.0, 51.4, 45.2, 40.4, 29.9, 25.1, 15.0. HRMS (HESI-TOF) m/z calcd for (M+H)⁺ 378.2287, found 378.2282.

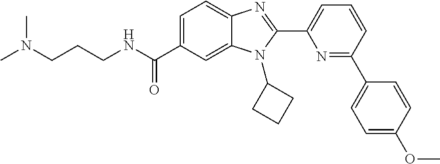

### 1-cyclobutyl-N-(3-(dimethylamino)propyl)-2-(6-(4-methoxyphenyl)pyridin-2-yl)-1H-benzo[d]imidazole-6-carboxamide

Molecular Formula: C₂₉H₃₃N₅O₂; yield 51%, off-white solid. ¹H NMR (600 MHz, CDCl₃) δ ppm 8.53 (s, 1H), 8.46 (s, 1H), 8.07 (dd, J=8.0, 3.8 Hz, 3H), 7.89 (t, J=7.8 Hz, 1H), 7.80 (dd, J=18.2, 8.2 Hz, 2H), 7.62 (d, J=8.3 Hz, 1H), 7.04 (d, J=8.7 Hz, 2H), 6.17-6.11 (m, 1H), 3.89 (s, 3H), 3.65 (dd, J=11.1, 5.5 Hz, 2H), 3.02-2.91 (m, 2H), 2.64-2.49 (m, 4H), 2.38 (s, 6H), 2.09-2.01 (m, 1H), 1.93-1.83 (m, 3H). ¹³C NMR (150 MHz, CDCl₃) δ ppm 167.7, 161.0, 156.0, 153.4, 150.2, 145.6, 137.8, 135.7, 131.4, 129.4, 128.2, 123.1, 120.4, 120.1, 119.8, 114.4, 113.0, 59.2, 55.5, 51.5, 45.3, 40.5, 29.9, 25.3, 15.1. HRMS (HESI-TOF) m/z calcd for (M+H)⁺ 484.2712, found 484.2707.

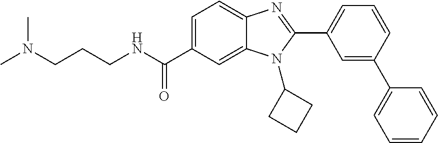 [SMILES: CN(C)CCCNC(=O)c1ccc2nc(-c3cccc(-c4ccccc4)c3)n(C3CCC3)c2c1]

### 2-([1,1′-biphenyl]-3-yl)-1-cyclobutyl-N-(3-(dimethylamino)propyl)-1H-benzo[d]imidazole-6-carboxamide

Molecular Formula: C₂₉H₃₂N₄O; yield 62%, white solid. ¹H NMR (600 MHz, CDCl₃) δ ppm 8.64 (s, 1H), 8.44 (s, 1H), 8.10 (s, 1H), 7.91-7.83 (m, 3H), 7.80 (d, J=8.4 Hz, 1H), 7.76-7.70 (m, 2H), 7.65-7.62 (m, 2H), 7.59 (d, J=6.4 Hz, 3H), 7.49-7.47 (m, 3H), 7.40-7.38 (m, 2H), 5.16-5.15 (m, 1H), 3.66 (d, J=5.4 Hz, 2H), 2.94 (dd, J=12.2, 10.0 Hz, 2H), 2.74 (s, 2H), 2.53-2.41 (m, 8H), 2.04 (d, J=10.4 Hz, 1H), 1.95 (s, 2H), 1.84-1.83 (m, 1H). ¹³C NMR (150 MHz, CDCl₃) δ ppm 168.4, 155.9, 146.0, 141.8, 140.3, 134.8, 131.4, 129.2, 129.1, 128.4, 128.3, 127.9, 127.3, 121.7, 119.8, 112.6, 55.7, 51.4, 43.3, 36.6, 29.8, 24.3, 15.0. HRMS (HESI-TOF) m/z calcd for (M+H)⁺ 453.2654, found 453.2646.

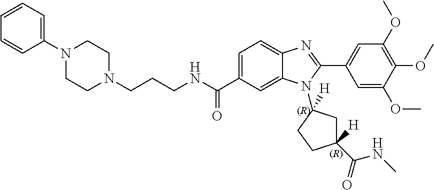

### 1-((1R,3R)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide

Molecular Formula: C₃₇H₄₆N₆O₅; yield 68%, white solid. ¹H NMR (600 MHz, CDCl₃) δ ppm 8.43 (s, 1H), 8.16 (s, 1H), 7.71 (t, J=12.1 Hz, 2H), 7.30-7.24 (m, 3H), 6.90 (t, J=9.4 Hz, 3H), 6.86 (s, 2H), 5.30 (t, J=8.5 Hz, 1H), 3.93 (d, J=8.4 Hz, 9H), 3.71-3.60 (m, 2H), 3.37-3.25 (m 5H), 2.78 (t, J=16.3 Hz, 8H), 2.59-2.51 (m, 1H), 2.48-2.46 (m, 2H), 2.27-2.22 (m, 1H), 2.17 (d, J=8.8 Hz, 1H), 1.94 (dd, J=11.8, 7.3 Hz, 2H). ¹³C NMR (150 MHz, CDCl₃) δ ppm 175.3, 167.8, 156.7, 153.6, 146.3, 139.9, 133.3, 129.4, 128.3, 125.4, 120.7, 119.9, 116.6, 112.3, 106.8, 61.1, 57.1, 56.4, 53.4, 49.2, 45.0, 33.8, 32.1, 31.7, 30.4, 29.8, 29.5, 26.5, 24.4, 22.8, 14.2. HRMS (HESI-TOF) m/z calcd for (M+H)⁺ 655.3607, found 655.3605.

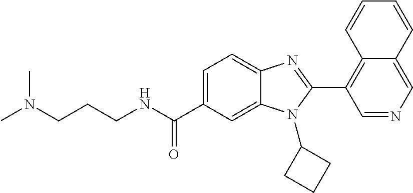 [SMILES: CN(C)CCCNC(=O)c1ccc2nc(-c3cncc4ccccc34)n(C3CCC3)c2c1]

### 1-cyclobutyl-N-(3-(dimethylamino)propyl)-2-(isoquinolin-4-yl)-1H-benzo[d]imidazole-6-carboxamide

Molecular Formula: C₂₆H₂₉N₅O₅; yield 60%, off-white solid. ¹H NMR (600 MHz, CDCl₃) δ ppm 9.40 (s, 1H), 8.67 (s, 2H), 8.46 (s, 1H), 8.10 (d, J=8.0 Hz, 1H), 7.86 (d, J=8.4 Hz, 1H), 7.77 (d, J=8.3 Hz, 1H), 7.73 (dd, J=7.4, 6.7 Hz, 1H), 7.69 (t, J=7.4 Hz, 1H), 7.66 (d, J=8.4 Hz, 1H), 4.82-4.74 (m, 1H), 3.65 (d, J=5.6 Hz, 2H), 2.89-2.82 (m, 2H), 2.60 (t, J=5.6 Hz, 2H), 2.38 (s, 6H), 2.28 (dd, J=18.2, 8.0 Hz, 2H), 1.95 (s, 1H), 1.88-1.83 (m, 2H), 1.74-1.65 (m, 1H). ¹³C NMR (150 MHz, CDCl₃) δ ppm 167.5, 154.5, 151.7, 146.1, 144.6, 134.8, 134.7, 131.9, 129.6, 128.3, 128.2, 124.6, 120.5, 120.1, 112.5, 59.3, 51.4, 45.4, 40.7, 29.7, 25.3, 15.0. HRMS (HESI-TOF) m/z calcd for (M+H)⁺428.2246, found 428.2441.

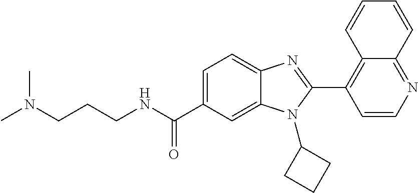 [SMILES: CN(C)CCCNC(=O)c1ccc2nc(-c3ccnc4ccccc34)n(C3CCC3)c2c1]

### 1-cyclobutyl-N-(3-(dimethylamino)propyl)-2-(quinolin-4-yl)-1H-benzo[d]imidazole-6-carboxamide

Molecular Formula: C₃₇H₄₆N₆O₅; yield 69%, white solid. ¹H NMR (600 MHz, CDCl₃) δ ppm 9.16 (s, 1H), 8.48 (s, 1H), 8.33 (d, J=8.3 Hz, 2H), 8.01 (d, J=8.2 Hz, 1H), 7.93 (d, J=8.3 Hz, 1H), 7.87 (t, J=7.5 Hz, 1H), 7.75 (d, J=8.3 Hz, 1H), 7.69 (d, J=3.1 Hz, 1H), 7.65 (t, J=7.5 Hz, 1H), 4.80-4.71 (m, 1H), 4.35 (s, 2H), 3.68 (d, J=4.1 Hz, 2H), 3.21 (s, 2H), 2.85 (s, 6H), 2.78 (s, 2H), 2.32-2.21 (m, 4H), 1.97-1.92 (m, 1H), 1.75-1.67 (m, 1H). ¹³C NMR (150 MHz, CDCl₃) δ ppm 168.2, 148.6, 131.3, 129.4, 128.8, 125.6, 122.6, 120.4, 112.3, 55.4, 51.7, 43.1, 36.6, 29.8, 24.3, 15.0. HRMS (HESI-TOF) m/z calcd for (M+H)⁺ 428.2447, found 428.2445.

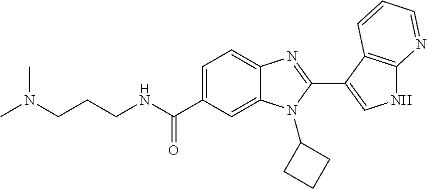 [SMILES: CN(C)CCCNC(=O)c1ccc2nc(-c3c[nH]c4ncccc34)n(C3CCC3)c2c1]

### 1-cyclobutyl-N-(3-(dimethylamino)propyl)-2-(1H-pyrrolo[2,3-b]pyridin-3-yl)-1H-benzo[d]imidazole-6-carboxamide

Molecular Formula: C₂₄H₂₈N₆O; yield 69%, white solid. ¹H NMR (600 MHz, CDCl₃) δ ppm 8.49 (s, 1H), 8.42 (s, 1H), 8.25 (d, J=7.4 Hz, 1H), 8.04 (s, 1H), 7.94 (d, J=8.0 Hz, 1H), 7.81 (d, J=7.8 Hz, 1H), 7.33 (s, 1H), 5.49-5.35 (m, 1H), 3.58 (t, J=6.4 Hz, 2H), 3.35 (s, 1H), 3.29-3.22 (m, 2H), 2.95 (s, 6H), 2.57 (d, J=8.5 Hz, 2H), 2.15-2.02 (m, 3H), 1.98-1.95 (m, 1H). ¹³C NMR (150 MHz, CDCl₃) δ ppm 170.5, 149.6, 145.4, 129.9, 123.7, 118.1, 113.9, 61.5, 56.8, 53.1, 43.6, 37.8, 30.3, 26.3, 20.8, 15.6, 14.5. HRMS (HESI-TOF) m/z calcd for (M+H)⁺ 417.2402, found 417.2403.

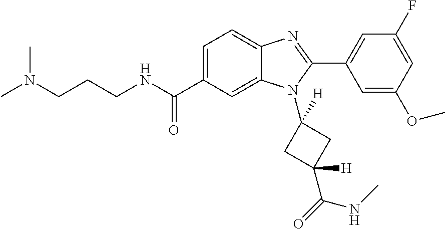

### N-(3-(dimethylamino)propyl)-2-(3-fluoro-5-methoxyphenyl)-1-((1r,3r)-3-(methyl carbamoyl)cyclobutyl)-1H-benzo[d]imidazole-6-carboxamide

Molecular Formula: C₂₆H₃₂FN₅O₃; yield 69%, white solid. ¹H NMR (600 MHz, CDCl₃) δ ppm 8.64 (s, 1H), 8.41 (s, 1H), 7.81 (d, J=8.4 Hz, 1H), 7.70 (d, J=8.2 Hz, 1H), 6.94 (s, 1H), 6.91 (d, J=8.2 Hz, 1H), 6.76 (d, J=10.4 Hz, 1H), 5.96 (s, 1H), 5.52-5.47 (m, 1H), 3.86 (s, 3H), 3.65 (d, J=5.1 Hz, 2H), 3.35 (s, 1H), 3.15 (dd, J=21.2, 10.3 Hz, 2H), 2.82 (d, J=4.6 Hz, 5H), 2.74 (s, 2H), 2.48 (s, 6H), 1.95 (bs, 2H). ¹³C NMR (150 MHz, CDCl₃) δ ppm 175.2, 167.6, 164.4, 162.7, 161.3, 161.2, 155.1, 146.0, 134.2, 132.5, 129.0, 121.1, 120.3, 111.9, 111.1, 109.1, 109.0, 104.0, 103.8, 57.9, 56.0, 49.4, 44.6, 39.3, 33.6, 32.0, 29.8, 26.6, 25.0. HRMS (HESI-TOF) m/z calcd for (M+H)⁺ 482.2567, found 482.2560.

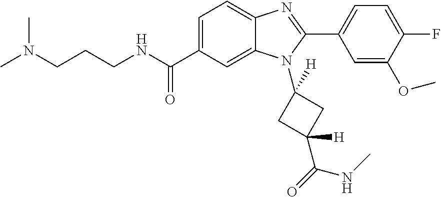 [SMILES: CNC(=O)[C@H]1C[C@H](n2c(-c3ccc(F)c(OC)c3)nc3ccc(C(=O)NCCCN(C)C)cc32)C1]

### N-(3-(dimethylamino)propyl)-2-(4-fluoro-3-methoxyphenyl)-1-((1r,3r)-3-(methyl carbamoyl)cyclobutyl)-1H-benzo[d]imidazole-6-carboxamide

Molecular Formula: C₂₆H₃₂FN₅O₃; yield 71%, white solid. ¹H NMR (600 MHz, CDCl₃) δ ppm 8.70 (s, 1H), 8.41 (s, 1H), 7.79 (d, J=8.4 Hz, 1H), 7.63 (d, J=8.3 Hz, 1H), 7.31 (d, J=6.9 Hz, 1H), 7.22 (dd, J=10.7, 8.4 Hz, 1H), 7.13-7.08 (m, 1H), 5.89 (s, 1H), 5.58-5.55 (m, 1H), 3.96 (s, 3H), 3.65 (d, J=5.6 Hz, 2H), 3.29 (s, 1H), 3.16 (d, J=11.0 Hz, 2H), 2.83 (d, J=4.7 Hz, 3H), 2.76 (d, J=10.1 Hz, 2H), 2.68 (s, 2H), 2.44 (s, 5H), 1.94-1.87 (m, 2H). ¹³C NMR (150 MHz, CDCl₃) δ ppm 175.3, 167.6, 155.5, 154.6, 152.9, 148.3, 148.2, 146.0, 134.3, 128.9, 126.7, 122.24, 122.2, 120.7, 120.1, 116.6, 116.5, 114.9, 58.7, 56.5, 49.5, 45.1, 40.0, 33.6, 32.2, 26.6, 25.1. HRMS (HESI-TOF) m/z calcd for (M+H)⁺ 482.2567, found 482.2552.

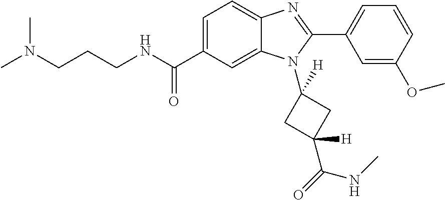

### N-(3-(dimethylamino)propyl)-2-(3-methoxyphenyl)-1-((1r,3r)-3-(methylcarbamoyl)cyclobutyl)-1H-benzo[d]imidazole-6-carboxamide

Molecular Formula: C₂₆H₃₃N₅O₃; yield 65%, white solid. ¹H NMR (600 MHz, CDCl₃) δ ppm 8.69 (s, 1H), 8.40 (s, 1H), 7.79 (d, J=8.4 Hz, 1H), 7.59 (d, J=8.4 Hz, 1H), 7.41 (t, J=7.9 Hz, 1H), 7.17 (s, 1H), 7.15 (d, J=7.5 Hz, 1H), 7.04 (dd, J=8.3, 1.9 Hz, 1H), 5.92 (s, 1H), 5.55-5.51 (m, 1H), 3.87 (s, 3H), 3.63 (d, J=5.6 Hz, 2H), 3.26 (s, 1H), 3.15-3.13 (m, 2H), 2.82 (d, J=4.7 Hz, 3H), 2.77 (t, J=10.1 Hz, 2H), 2.62 (s, 2H), 2.40 (s, 5H), 1.90-1.84 (m, 2H). ¹³C NMR (150 MHz, CDCl₃) δ ppm 175.3, 167.6, 159.9, 156.3, 146.1, 134.3, 131.5, 130.0, 128.9, 121.8, 120.4, 120.1, 116.6, 114.7, 112.1, 59.0, 49.4, 45.2, 40.4, 33.6, 32.1, 26.6, 25.2. HRMS (HESI-TOF) m/z calcd for (M+H)⁺ 464.2661, found 464.2654.

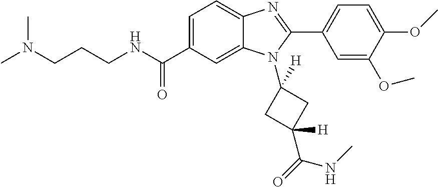

### 2-(3,4-dimethoxyphenyl)-N-(3-(dimethylamino)propyl)-1-((1r,3r)-3-(methylcarbamoyl)cyclobutyl)-1H-benzo[d]imidazole-6-carboxamide

Molecular Formula: C₂₇H₃₅N₅O₄; yield 62%, white solid. ¹H NMR (600 MHz, CDCl₃) δ ppm 8.59 (s, 1H), 8.39 (s, 1H), 7.94 (d, J=8.3 Hz, 1H), 7.83 (d, J=8.4 Hz, 1H), 7.24 (s, 1H), 7.12 (d, J=8.2 Hz, 1H), 7.00 (t, J=9.2 Hz, 1H), 6.35 (s, 1H), 5.56-5.52 (m, 1H), 3.96 (d, J=2.0 Hz, 6H), 3.72 (d, J=5.8 Hz, 3H), 3.19 (dd, J=21.5, 10.4 Hz, 2H), 3.05 (s, 2H), 2.85 (s, 5H), 2.74 (s, 6H), 2.18 (s, 2H). ¹³C NMR (150 MHz, CDCl₃) δ ppm 175.3, 167.7, 161.1, 146.1, 134.2, 131.9, 128.7, 120.2, 111.8, 107.6, 102.7, 55.7, 49.4, 44.9, 33.5, 31.9, 26.6, 25.0. HRMS (HESI-TOF) m/z calcd for (M+H)⁺ 494.2767, found 494.2762.

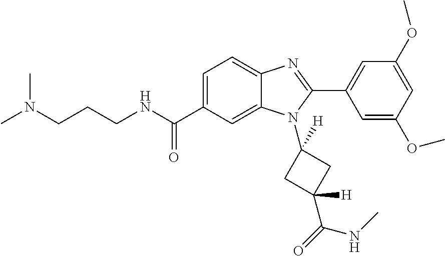

### 2-(3,5-dimethoxyphenyl)-N-(3-(dimethylamino)propyl)-1-((1r,3r)-3-(methylcarbamoyl)cyclobutyl)-1H-benzo[d]imidazole-6-carboxamide

Molecular Formula: C₂₇H₃₅N₅O₄; yield 70%, white solid. ¹H NMR (600 MHz, CDCl₃) δ ppm 8.65 (s, 1H), 8.40 (s, 1H), 7.81 (d, J=8.4 Hz, 1H), 7.67 (d, J=8.3 Hz, 1H), 6.73 (d, J=2.0 Hz, 2H), 6.59 (s, 1H), 5.95 (s, 1H), 5.55-5.52 (m, 1H), 3.85 (s, 6H), 3.65 (dd, J=10.9, 5.4 Hz, 2H), 3.40 (s, 1H), 3.15 (dd, J=21.5, 10.4 Hz, 2H), 2.83 (d, J=4.8 Hz, 5H), 2.72 (s, 2H), 2.48 (s, 6H), 1.94 (s, 2H). ¹³C NMR (150 MHz, CDCl₃) δ ppm 175.3, 167.7, 161.1, 146.1, 134.2, 131.9, 128.7, 120.2, 111.8, 107.6, 102.8, 55.7, 49.4, 44.9, 33.6, 31.9, 26.6, 25.0. HRMS (HESI-TOF) m/z calcd for (M+H)⁺ 494.2767, found 494.2762.

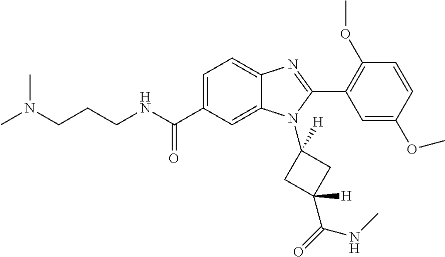

### 2-(2,5-dimethoxyphenyl)-N-(3-(dimethylamino)propyl)-1-((1r,3r)-3-(methylcarbamoyl)cyclobutyl)-1H-benzo[d]imidazole-6-carboxamide

Molecular Formula: C₂₇H₃₅N₅O₄; yield 72%, white solid. ¹H NMR (600 MHz, CDCl₃) δ ppm 8.63 (s, 1H), 8.40 (s, 1H), 7.81 (d, J=8.4 Hz, 1H), 7.64 (d, J=8.3 Hz, 1H), 7.09 (d, J=3.0 Hz, 1H), 7.03 (dd, J=9.0, 3.1 Hz, 1H), 6.93 (d, J=9.1 Hz, 1H), 5.95 (s, 1H), 5.14-5.08 (m, 1H), 3.80 (s, 3H), 3.75 (s, 3H), 3.67-3.63 (m, 2H), 3.38 (s, 1H), 3.11 (d, J=7.7 Hz, 2H), 2.81 (d, J=4.8 Hz, 3H), 2.71 (s, 2H), 2.46 (s, 6H), 1.95-1.91 (m, 2H). ¹³C NMR (150 MHz, CDCl₃) δ ppm 175.6, 167.8, 154.1, 153.8, 151.7, 146.4, 133.8, 128.5, 120.4, 120.1, 119.9, 117.9, 116.8, 112.3, 111.5, 58.4, 56.1, 49.6, 44.9, 39.6, 33.7, 31.8, 26.6, 25.0. HRMS (HESI-TOF) m/z calcd for (M+H)⁺ 494.2767, found 494.2759.

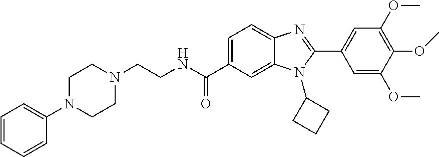 [SMILES: COc1cc(-c2nc3ccc(C(=O)NCCN4CCN(c5ccccc5)CC4)cc3n2C2CCC2)cc(OC)c1OC]

### 1-cyclobutyl-N-(2-(4-phenylpiperazin-1-yl)ethyl)-2-(3,4,5-trimethoxyphenyl)-1H-benzo[d]imidazole-6-carboxamide

Molecular Formula: C₃₃H₃₉N₅O₄; yield 75%, white solid. ¹H NMR (600 MHz, CDCl₃) δ ppm 8.41 (s, 1H), 7.80 (d, J=8.4 Hz, 1H), 7.64 (d, J=8.3 Hz, 1H), 7.28 (d, J=8.4 Hz, 2H), 6.94 (d, J=8.0 Hz, 2H), 6.88 (t, J=7.3 Hz, 1H), 6.85 (s, 2H), 5.17-5.13 (m, 1H), 3.92 (d, J=6.1 Hz, 9H), 3.73-3.67 (m, 2H), 3.29 (s, 4H), 3.01-2.92 (m, 2H), 2.79 (s, 6H), 2.44 (dd, J=18.3, 8.4 Hz, 2H), 2.07-2.05 (m, 1H), 1.88-1.86 (m, 1H). ¹³C NMR (150 MHz, CDCl₃) δ ppm 167.8, 156.1, 153.5, 151.1, 145.9, 139.8, 134.9, 129.3, 128.6, 125.9, 120.4, 120.3, 119.9, 116.3, 112.6, 107.0, 61.2, 56.8, 56.5, 53.1, 51.4, 49.2, 36.5, 29.7, 14.9. HRMS (HESI-TOF) m/z calcd for (M+H)⁺ 570.3080, found 570.3083.

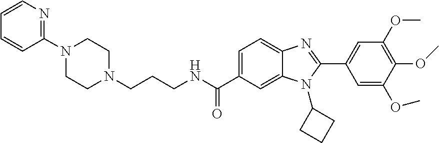 [SMILES: COc1cc(-c2nc3ccc(C(=O)NCCCN4CCN(c5ccccn5)CC4)cc3n2C2CCC2)cc(OC)c1OC]

### 1-cyclobutyl-N-(3-(4-(pyridin-2-yl)piperazin-1-yl)propyl)-2-(3,4,5-trimethoxy phenyl)-1H-benzo[d]imidazole-6-carboxamide

Molecular Formula: C₃₃H₄₀N₆O₄; yield 80%, white solid. ¹H NMR (600 MHz, CDCl₃) δ ppm 8.42 (s, 1H), 8.20-8.14 (m, 2H), 7.69 (d, J=8.4 Hz, 1H), 7.63 (d, J=8.2 Hz, 1H), 7.52-7.46 (m, 1H), 6.83 (s, 2H), 6.66-6.61 (m, 2H), 5.15-5.09 (m, 1H), 3.92 (d, J=10.0 Hz, 9H), 3.70-3.63 (m, 2H), 3.58 (s, 4H), 3.02-2.90 (m, 2H), 2.67 (d, J=5.0 Hz, 6H), 2.43 (dd, J=18.3, 8.4 Hz, 2H), 2.07-2.04 (m, 2H), 1.96-1.82 (m, 4H). ¹³C NMR (150 MHz, CDCl₃) δ ppm 167.9, 159.5, 156.0, 153.5, 148.1, 145.8, 139.8, 137.7, 135.0, 129.1, 126.0, 120.3, 119.6, 113.9, 112.8, 107.2, 107.0, 61.2, 58.4, 56.5, 53.4, 51.4, 45.4, 40.8, 29.6, 24.8, 14.9. HRMS (HESI-TOF) m/z calcd for (M+H)⁺ 585.3189, found 585.3184.

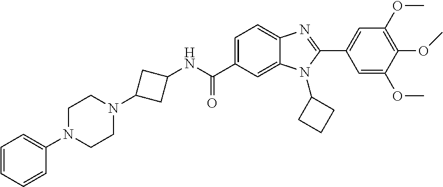 [SMILES: COc1cc(-c2nc3ccc(C(=O)NC4CC(N5CCN(c6ccccc6)CC5)C4)cc3n2C2CCC2)cc(OC)c1OC]

### 1-cyclobutyl-N-(3-(4-phenylpiperazin-1-yl)cyclobutyl)-2-(3,4,5-trimethoxyphenyl)-1H-benzo[d]imidazole-6-carboxamide

Molecular Formula: C₃₃H₄₀N₆O₄; yield 80%, colorless liquid. ¹H NMR (600 MHz, CDCl₃) δ ppm 8.40 (s, 1H), 7.78 (d, J=8.4 Hz, 1H), 7.58 (d, J=8.4 Hz, 1H), 7.26 (s, 2H), 6.93 (d, J=8.0 Hz, 2H), 6.87-6.82 (m, 3H), 6.64 (d, J=6.2 Hz, 1H), 5.16-5.10 (m, 1H), 4.58 (d, J=5.4 Hz, 1H), 3.92 (d, J=8.5 Hz, 9H), 3.25-3.18 (m, 4H), 3.07 (d, J=7.0 Hz, 1H), 2.98-2.89 (m, 2H), 2.58-2.50 (m, 6H), 2.43 (dd, J=18.3, 8.5 Hz, 2H), 2.26-2.17 (m, 2H), 2.04 (d, J=10.6 Hz, 1H), 1.92-1.81 (m, 1H). ¹³C NMR (150 MHz, CDCl₃) δ ppm 167.6, 156.2, 153.5, 151.4, 145.9, 139.9, 135.0, 129.2, 128.6, 125.8, 119.9, 119.9, 119.7, 116.2, 112.8, 107.0, 61.1, 57.1, 56.4, 51.3, 50.1, 49.1, 42.6, 34.0, 29.6, 14.9. HRMS (HESI-TOF) m/z calcd for (M+H)⁺ 596.3237, found 596.3243.

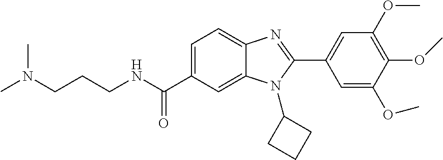

### 1-cyclobutyl-N-(3-(dimethylamino)propyl)-2-(3,4,5-trimethoxyphenyl)-1H-benzo[d]imidazole-6-carboxamide

Molecular Formula: C₂₆H₃₄N₄O₄; yield 82%, colorless liquid. ¹H NMR (600 MHz, CDCl₃) δ ppm 8.56 (s, 1H), 8.42 (s, 1H), 7.78 (d, J=8.4 Hz, 1H), 7.55 (d, J=8.4 Hz, 1H), 6.85 (s, 2H), 5.17-5.12 (m, 1H), 3.93 (d, J=5.1 Hz, 9H), 3.64 (dd, J=11.1, 5.6 Hz, 2H), 3.04-2.95 (m, 2H), 2.54 (t, J=5.8 Hz, 2H), 2.45 (dd, J=18.3, 8.4 Hz, 2H), 2.33 (s, 6H), 2.08-2.06 (m, 1H), 1.92-1.72 (m, 7H). ¹³C NMR (150 MHz, CDCl₃) δ ppm 167.4, 155.8, 153.4, 145.6, 139.7, 134.8, 129.1, 125.9, 120.1, 119.6, 112.5, 106.9, 61.1, 59.6, 56.4, 51.3, 45.6, 401.0, 29.5, 25.4, 14.8. HRMS (HESI-TOF) m/z calcd for (M+H)⁺ 467.2658, found 467.2643.

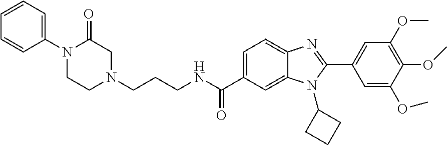 [SMILES: COc1cc(-c2nc3ccc(C(=O)NCCCN4CCN(c5ccccc5)C(=O)C4)cc3n2C2CCC2)cc(OC)c1OC]

### 1-cyclobutyl-N-(3-(3-oxo-4-phenylpiperazin-1-yl)propyl)-2-(3,4,5-trimethoxyphenyl)-1H-benzo[d]imidazole-6-carboxamide

Molecular Formula: C₂₆H₃₄N₄O₄; yield 80%, white solid. ¹H NMR (600 MHz, CDCl₃) δ ppm 8.40 (s, 1H), 7.75 (d, J=8.4 Hz, 1H), 7.61-7.53 (m, 2H), 7.39 (t, J=7.8 Hz, 2H), 7.26 (d, J=3.3 Hz, 2H), 7.22 (d, J=7.5 Hz, 2H), 6.83 (s, 2H), 5.14-5.08 (m, 1H), 3.91 (d, J=9.8 Hz, 9H), 3.66 (s, 4H), 3.38 (s, 2H), 2.98-2.90 (m, 2H), 2.87 (t, J=5.3 Hz, 2H), 2.68 (t, J=6.2 Hz, 2H), 2.42 (dd, J=18.2, 8.4 Hz, 2H), 2.03 (dd, J=20.7, 10.3 Hz, 1H), 1.94-1.82 (m, 4H). ¹³C NMR (150 MHz, CDCl₃) δ ppm 167.9, 166.6, 156.1, 153.5, 145.8, 141.8, 139.8, 135.0, 129.4, 129.0, 127.2, 125.9, 119.8, 112.9, 107.0, 61.1, 58.3, 56.9, 56.4, 51.3, 50.2, 50.2, 40.0, 29.6, 25.6, 14.9. HRMS (HESI-TOF) m/z calcd for (M+H)⁺ 598.3029 found 598.3023.

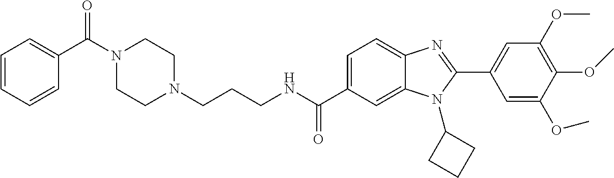 [SMILES: COc1cc(-c2nc3ccc(C(=O)NCCCN4CCN(C(=O)c5ccccc5)CC4)cc3n2C2CCC2)cc(OC)c1OC]

### N-(3-(4-benzoylpiperazin-1-yl)propyl)-1-cyclobutyl-2-(3,4,5-trimethoxyphenyl)-1H-benzo[d]imidazole-6-carboxamide

Molecular Formula: C₃₅H₄₁N₅O₅; yield 84%, white solid. ¹H NMR (600 MHz, CDCl₃) δ ppm 8.39 (s, 1H), 7.77 (d, J=8.4 Hz, 1H), 7.69 (s, 1H), 7.56 (d, J=8.3 Hz, 1H), 7.39 (dd, J=8.1, 4.4 Hz, 5H), 6.85 (s, 2H), 5.16-5.11 (m, 1H), 3.92 (d, J=6.8 Hz, 9H), 3.83-3.76 (m, 1H), 3.62 (dd, J=11.6, 5.9 Hz, 2H), 3.42 (s, 2H), 3.03-2.87 (m, 4H), 2.60 (t, J=6.1 Hz, 4H), 2.44 (dd, J=18.1, 8.4 Hz, 3H), 2.10-2.01 (m, 2H), 1.86 (dd, J=12.1, 6.3 Hz, 3H). ¹³C NMR (150 MHz, CDCl₃) δ ppm 170.4, 167.9, 156.1, 153.5, 145.8, 139.9, 135.7, 135.1, 129.9, 129.1, 128.6, 127.1, 125.9, 119.9, 119.6, 112.9, 107.0, 61.1, 57.7, 56.5, 51.3, 40.3, 29.6, 25.3, 14.9. HRMS (HESI-TOF) m/z calcd for (M+H)⁺612.3186 found 612.3187.

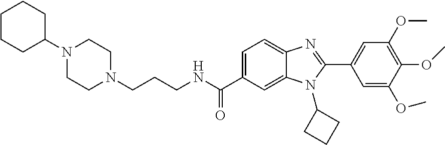 [SMILES: COc1cc(-c2nc3ccc(C(=O)NCCCN4CCN(C5CCCCC5)CC4)cc3n2C2CCC2)cc(OC)c1OC]

### 1-cyclobutyl-N-(3-(4-cyclohexylpiperazin-1-yl)propyl)-2-(3,4,5-trimethoxyphenyl)-1H-benzo[d]imidazole-6-carboxamide

Molecular Formula: C₃₄H₄₇N₅O₄; yield 82%, white solid. ¹H NMR (600 MHz, CDCl₃) δ ppm 8.47 (s, 1H), 8.43 (bs, 1H), 7.76 (d, J=8.4 Hz, 1H), 7.67 (d, J=8.3 Hz, 1H), 6.84 (s, 2H), 5.17-5.11 (m, 1H), 3.92 (d, J=5.8 Hz, 9H), 3.62 (d, J=5.5 Hz, 2H), 3.05-2.93 (m, 2H), 2.75 (bs, 5H), 2.67-2.60 (m, 3H), 2.44 (dd, J=18.3, 8.5 Hz, 2H), 2.36 (s, 1H), 2.08-2.06 (m, 1H), 1.93-1.78 (m, 7H), 1.63 (d, J=12.5 Hz, 1H), 1.23 (d, J=10.3 Hz, 6H), 1.14-1.04 (m, 1H). ¹³C NMR (150 MHz, CDCl₃) δ ppm 167.7, 156.0, 153.5, 145.8, 139.8, 135.0, 128.9, 126.0, 120.5, 119.5, 113.0, 107.0, 63.9, 61.1, 58.1, 56.4, 53.4, 51.4, 48.6, 40.8, 29.6, 28.7, 26.2, 25.8, 24.3, 14.9. HRMS (HESI-TOF) m/z calcd for (M+H)⁺ 590.3706 found 590.3702.

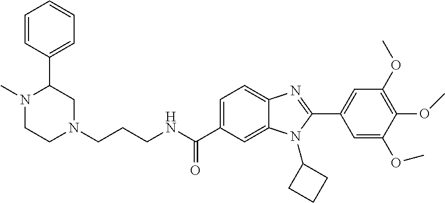

### 1-cyclobutyl-N-(3-(4-methyl-3-phenylpiperazin-1-yl)propyl)-2-(3,4,5-trimethoxy phenyl)-1H-benzo[d]imidazole-6-carboxamide

Molecular Formula: C₃₅H₄₃N₅O₄; yield 99%, white solid. ¹H NMR (600 MHz, CDCl₃) δ ppm 8.43 (s, 1H), 8.34 (s, 1H), 7.83 (d, J=8.4 Hz, 1H), 7.73 (d, J=8.4 Hz, 1H), 7.23 (dd, J=13.1, 6.2 Hz, 5H), 6.85 (s, 2H), 5.18-5.12 (m, 1H), 3.91 (d, J=5.5 Hz, 9H), 3.64-3.60 (m, 2H), 3.06 (d, J=10.4 Hz, 2H), 3.01-2.90 (m, 4H), 2.63 (m, 2H), 2.51-2.35 (m, 4H), 2.16 (t, J=10.9 Hz, 1H), 2.03 (s, 4H), 1.92-1.78 (m, 3H). ¹³C NMR (150 MHz, CDCl₃) δ ppm 168.0, 156.0, 153.5, 145.9, 140.8, 139.8, 135.0, 128.9, 128.6, 127.9, 125.929, 120.7, 119.6, 112.9, 106.9, 69.0, 61.2, 61.1, 57.9, 56.4, 55.6, 53.5, 51.4, 43.5, 40.7, 29.6, 24.5, 14.9. HRMS (HESI-TOF) m/z calcd for (M+H)⁺ 598.3393 found 598.3394.

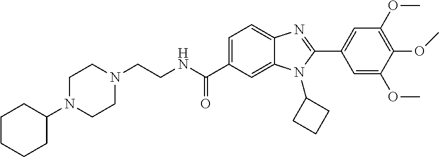 [SMILES: COc1cc(-c2nc3ccc(C(=O)NCCN4CCN(C5CCCCC5)CC4)cc3n2C2CCC2)cc(OC)c1OC]

### 1-cyclobutyl-N-(2-(4-cyclohexylpiperazin-1-yl)ethyl)-2-(3,4,5-trimethoxyphenyl)-1H-benzo[d]imidazole-6-carboxamide

Molecular Formula: C₃₃H₄₅N₅O₄; yield 83%, colorless liquid. ¹H NMR (600 MHz, CDCl₃) δ ppm 8.36 (s, 1H), 7.78 (d, J=8.4 Hz, 1H), 7.60 (d, J=8.3 Hz, 1H), 7.11 (s, 1H), 6.83 (s, 2H), 5.16-5.10 (m, 1H), 3.90 (d, J=4.2 Hz, 9H), 3.63-3.55 (m, 2H), 3.43 (s, 1H), 2.99-2.89 (m, 2H), 2.64-2.59 (m, 5H), 2.55 (s, 2H), 2.45-2.39 (m, 3H), 2.21 (dd, J=14.2, 6.5 Hz, 1H), 2.05-2.03 (m, 1H), 1.86 (d, J=10.1 Hz, 3H), 1.77 (d, J=11.6 Hz, 2H), 1.60 (d, J=12.8 Hz, 1H), 1.27-1.14 (m, 5H), 1.09-1.07 (m, 1H). ¹³C NMR (150 MHz, CDCl₃) δ ppm 167.7, 156.0, 153.5, 145.8, 139.8, 134.9, 128.9, 125.9, 120.3, 119.8, 112.6, 106.9, 63.6, 61.1, 56.6, 56.4, 53.5, 51.3, 49.1, 36.6, 29.6, 29.1, 26.4, 25.9, 14.9. HRMS (HESI-TOF) m/z calcd for (M+H)⁺ 576.3550 found 576.3558.

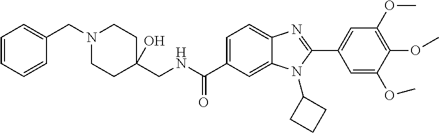 [SMILES: COc1cc(-c2nc3ccc(C(=O)NCC4(O)CCN(Cc5ccccc5)CC4)cc3n2C2CCC2)cc(OC)c1OC]

### N-((1-benzyl-4-hydroxypiperidin-4-yl)methyl)-1-cyclobutyl-2-(3,4,5-trimethoxyphenyl)-1H-benzo[d]imidazole-6-carboxamide

Molecular Formula: C₃₄H₄₀N₄O₅; yield 75%, white solid. ¹H NMR (600 MHz, CDCl₃) δ ppm 8.39 (s, 1H), 7.83 (s, 1H), 7.78 (d, J=8.4 Hz, 1H), 7.73 (d, J=8.4 Hz, 1H), 7.42 (d, J=6.7 Hz, 2H), 7.35-7.28 (m, 3H), 6.81 (s, 2H), 5.10-5.04 (m, 1H), 3.89 (d, J=13.8 Hz, 9H), 3.77 (s, 2H), 3.55 (d, J=5.6 Hz, 2H), 2.93-2.83 (m, 4H), 2.77 (d, J=10.3 Hz, 2H), 2.39-2.37 (m, 2H), 2.02-1.97 (m, 2H), 1.91 (t, J=10.8 Hz, 2H), 1.83-1.73 (m, 3H). ¹³C NMR (150 MHz, CDCl₃) δ ppm 169.9, 156.2, 153.4, 146.1, 139.8, 134.8, 130.2, 128.8, 128.5, 127.9, 125.8, 121.1, 119.7, 112.8, 106.9, 69.3, 62.1, 61.1, 56.4, 51.3, 50.6, 49.1, 34.1, 29.7, 14.9. HRMS (HESI-TOF) m/z calcd for (M+H)⁺ 585.3077 found 585.3084.

### Example 3: Evaluation of Hit Compounds

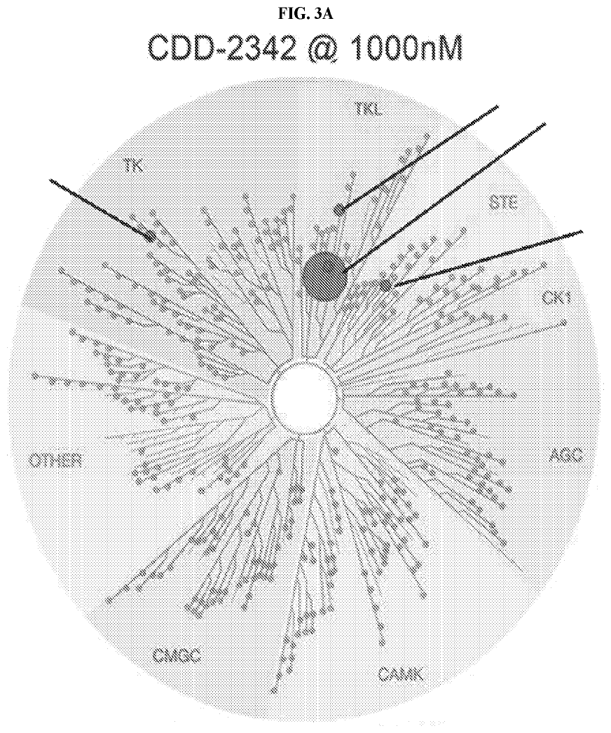

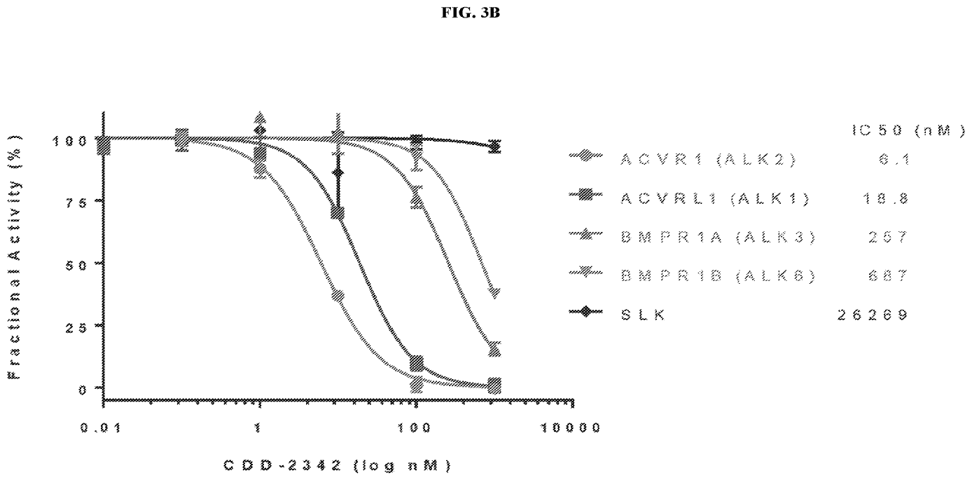

Synthesis and evaluation of these hits off-DNA indicated that the compounds disclosed herein are nanomolar potent inhibitors with specificity for ACVR1 over other related and unrelated kinases. In particular, CDD-2342 has an in vitro IC₅₀ of 6.1 nM for ACVR1 and shows weaker inhibition of ACVRL1 (18.8 nM), BMPR1A (257 nM) and BMPR1B (687 nM). Additionally, CDD-2342 is a >1000-fold better inhibitor of ACVR1 than non-TGFβ family kinases (FIGS. 3A-3B).

Two related series of compounds were identified using the DEC-Tec platform, one of which is similarly active toward ACVR1 and ACVRL1 and the other that shows up to 19-fold specificity in kinase assays toward ACVR1 versus ACVRL1 (Table 2).

TABLE 2
Inhibition of ACVR1 and ACVRL1 (ThermoFisher Assay)

| Compound | ACVR1 IC₅₀ (nM) | ACVRL1 IC₅₀ (nM) |
|---|---|---|
| 1 (CDD-2205) | 3.7 | 14.8 |
| 2 (CDD-2206) | 5.3 | 26.9 |
| 3 (CDD-2233) | 19.9 | 20.1 |
| 4 (CDD-2234) | 79.7 | 70.3 |
| 5 (CDD-2235) | 5.1 | 17.5 |
| 6 (CDD-2236) | 65.6 | 534 |
| 7 (CDD-2279) | 1988 | 1270 |
| 8 (CDD-2280) | 23.1 | 30.7 |
| 9 (CDD-2281) | 14.6 | 15.6 |
| 10 (CDD-2282) | 16.4 | 16.5 |
| 11 (CDD-2313) | 16 | 198 |
| 12 (CDD-2314) | 7.3 | 32.5 |
| 13 (CDD-2315) | 4.2 | 10.9 |
| 14 (CDD-2316) | 129 | 807 |
| 15 (CDD-2317) | 143 | 2059 |
| 16 (CDD-2339) | 83 | 839 |
| 17 (CDD-2340) | 20.5 | 390 |
| 18 (CDD-2341) | 3.6 | 12.3 |
| 19 (CDD-2342) | 6.1 | 18.8 |
| 20 (CDD-2420) | 4.9 | 24.1 |
| 21 (CDD-2421) | 7.9 | 77 |
| 22 (CDD-2464) | 202 | 131 |
| 23 (CDD-2465) | 44 | 42 |
| 24 (CDD-2467) | 13 | 117 |
| 25 (CDD-2468) | 135 | 4048 |
| 26 (CDD-2469) | 17.4 | 124 |
| 27 (CDD-2617) | 49 | 170 |
| 28 (CDD-2618) | 725 | 2140 |
| 29 (CDD-2619) | 480 | 1365 |
| 30 (CDD-2620) | 1857 | NA |
| 31 (CDD-2621) | 3811 | NA |
| 32 (CDD-2622) | 5.7 | 11.5 |
| 33 (CDD-2623) | 1850 | NA |
| 34 (CDD-2624) | 232 | 719 |
| 35 (CDD-2625) | 16.3 | 19.8 |
| 36 (CDD-2682) | 87.1 | 407 |
| 37 (CDD-2683) | 77.4 | 190 |
| 38 (CDD-2684) | 84 | 197 |
| 39 (CDD-2685) | 121 | 390 |
| 40 (CDD-2686) | 34.3 | 72.2 |
| 41 (CDD-2687) | 25.1 | 78.7 |
| 42 (CDD-2786) | 22.5 | 18.6 |
| 43 (CDD-2787) | 3.8 | 9.7 |
| 44 (CDD-2788) | 24.8 | 13.2 |
| 45 (CDD-2789) | 6.4 | 9.9 |
| 46 (CDD-2790) | 8.5 | 40.8 |
| 47 (CDD-2791) | 7.9 | 14.6 |
| 48 (CDD-2792) | 4.1 | 16.7 |
| 49 (CDD-2793) | 9.0 | 9.2 |
| 50 (CDD-2794) | 10.2 | 11.2 |
| 51 (CDD-2795) | 3.4 | 4.2 |

NA, not active at 1 μM; ND, not determined.

Exemplary compounds of the present disclosure have been evaluated for metabolic stability in mouse and human liver microsomes. The results of the metabolic studies described herein demonstrate that several compounds are stable for at least 12 h with exposure to human liver microsomes (Table 3).

TABLE 3
Activities of CDD-2342 and analogues thereof

| | | NanoBRET | | | |
|---|---|---|---|---|---|
| | NanoBRET | ACVR1 | NanoBRET | | |
| | ACVR1 | (R206H) | ACVRL1 | Metabolism | Metabolism |
| Compound | IC₅₀ (μM) | IC₅₀ (μM) | IC₅₀ (μM) | MLM (Min) | HLM (Min) |
| 1 (CDD-2235) | — | — | — | 5.7 | 9.7 |
| 2 (CDD-2315) | 0.56 | 2.6 | NI | 6.3 | 7.8 |
| 3 (CDD-2342) | 0.81 | 1.48 | 11.37 | 0 | 0.5 |
| 4 (CDD-2280) | NI | NI | NI | 48 | 70.7 |
| 5 (CDD-2281) | NI | NI | NI | 807.1 (13.4 h) | 769.1 (12.81 h) |
| 6 (CDD-2282) | NI | NI | NI | 220.4 (3.67 h) | 978 (16.3 h) |
| 7 (CDD-2467) | — | — | — | 9.3 | 13.6 |
| 8 (CDD-2313) | 6.17 | 3.4 | NI | 8.6 | 23 |
| 9 (CDD-2340) | 5.3 | 9.2 | NI | 7.1 | 24.4 |
| 10 (CDD-2421) | 1.24 | 2.4 | 38 | 3.7 | 6 |
| 11 (CDD-2469) | 4.72 | 4.45 | 34 | — | — |
| 12 (CDD-2617) | 10.7 | 13.4 | NI | 78.5 (1.3 h) | 194 (3.2 h) |
| 13 (CDD-2686) | 28.2 | 29.6 | NI | — | — |
| 14 (CDD-2687) | NI | NI | NI | — | — |
| 43 (CDD-2787) | 0.473 | 0.705 | 36.72 | 3.3 | 9.8 |
| 45 (CDD-2789) | 0.537 | 1.128 | 10.07 | 505 (8.4 h) | 2767 (46.1 h) |
| 46 (CDD-2790) | 1.822 | 2.268 | NI | 6.1 | 9.0 |
| 47 (CDD-2791) | 1.08 | 2.605 | NI | 2.8 | 15.4 |
| 48 (CDD-2792) | 0.358 | 0.849 | 28.96 | 10 | 38.3 |
| 49 (CDD-2793) | 2.718 | 4.281 | 32.64 | — | — |
| 50 (CDD-2794) | 2.087 | 3.164 | NI | — | — |
| 51 (CDD-2795) | 0.387 | 0.459 | 8.55 | — | — |

NI = No inhibition at 40 μM

### Example 4: Cell-Based Evaluation of Compounds as Inhibitors of ACVR1^{WT}, ACVR1^{R206H}, and ACVRL1

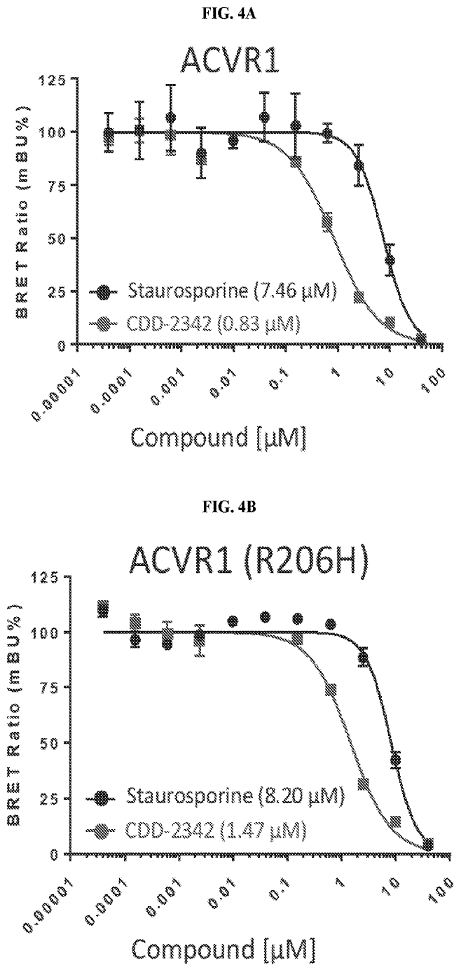

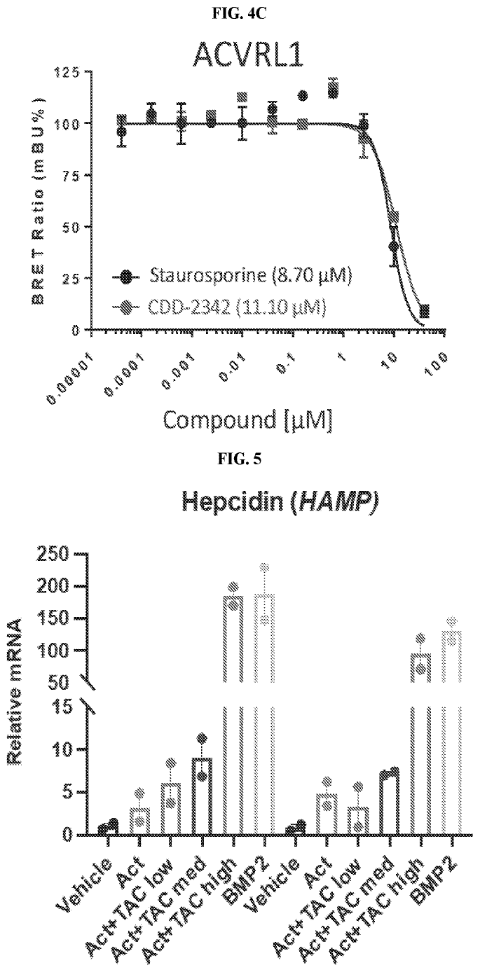

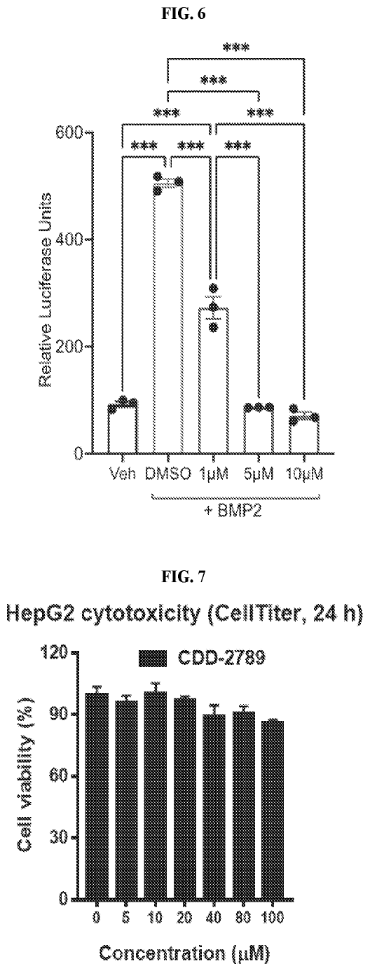

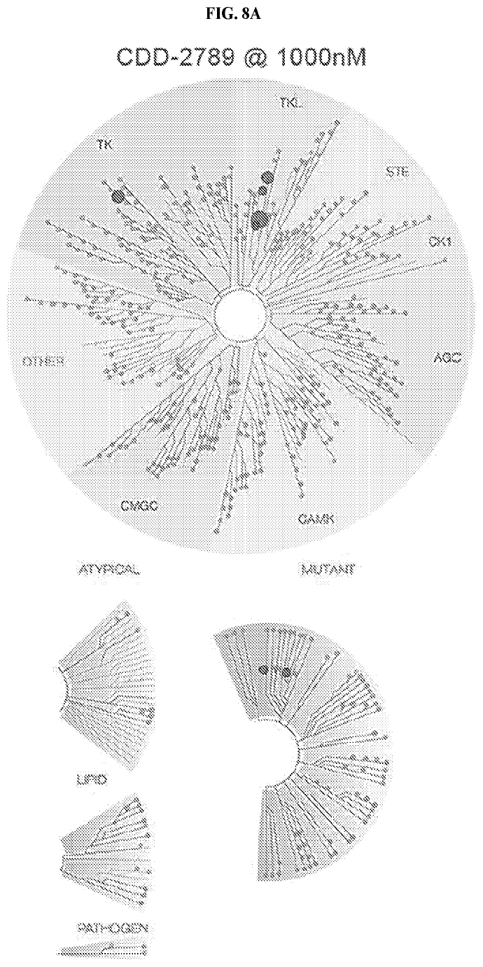

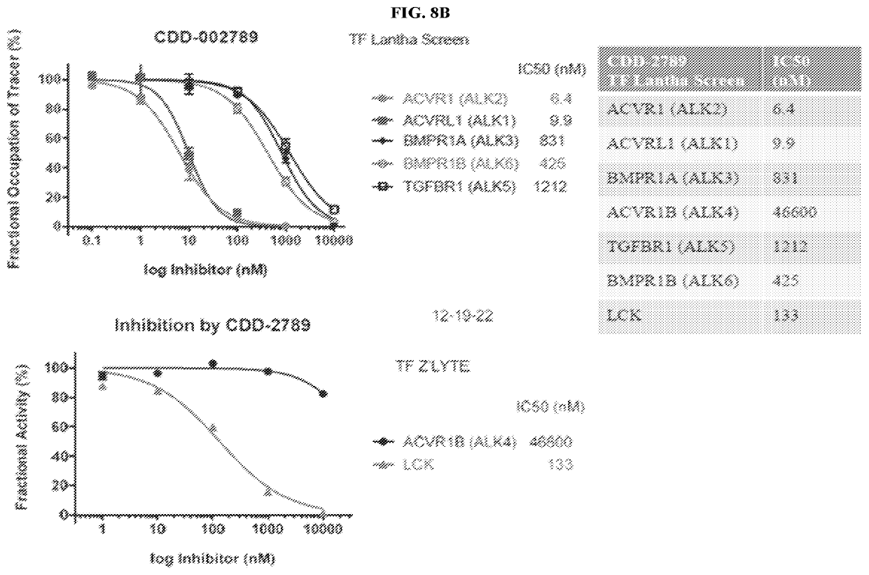

The present disclosure further provides exemplary data for the inhibition of ACVR1^{WT}, ACVR1^{R206H}, and ACVRL1 in cells. In certain embodiments, the kinase inhibitor assays described herein comprise Promega NanoLuc luciferase fusion constructs for ACVR1^{WT}, ACVR1^{R206H}, and ACVRL1. Thus, the in cellulo assays described herein comprise a luciferase reporter cell line system. In certain embodiments, the exemplary data was obtained utilizing CDD-2342 as the kinase inhibitor in the assays described herein (FIGS. 4A-4C). In certain embodiments, Staurosporine (i.e., non-selective kinase inhibitor) is used a control in the experiments described herein.

In certain embodiments, CDD-2342 (IC₅₀=0.83 μM) was found to be more potent than Staurosporine (IC₅₀=7.46 μM) against ACVR1^{WT} in cellulo (FIG. 4A). In certain embodiments, CDD-2342 (IC₅₀=1.47 μM) was found to be more potent than Staurosporine (IC₅₀=8.20 μM) against ACVR1^{R206H} in cellulo (FIG. 4B). In certain embodiments, CDD-2342 (IC₅₀=11.10 μM) was found to be less potent than Staurosporine (IC₅₀=8.70 μM) against ACVR1^{WT} in cellulo (FIG. 4C). Thus, the experiments described herein indicate CDD-2342 is a more potent inhibitor of ACVR1^{WT} and ACVR1^{R206H} than Staurosporine, and possesses selectivity for ACVR1^{WT} and ACVR1^{R206H} over ACVR1L, which Staurosporine clearly does not.

### Example 5: Pharmacokinetic Studies

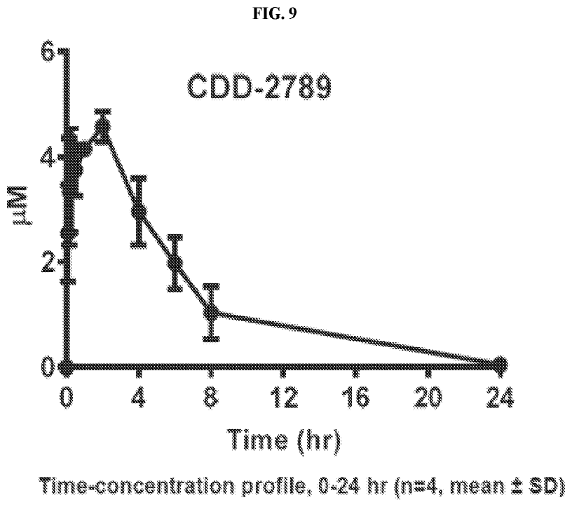

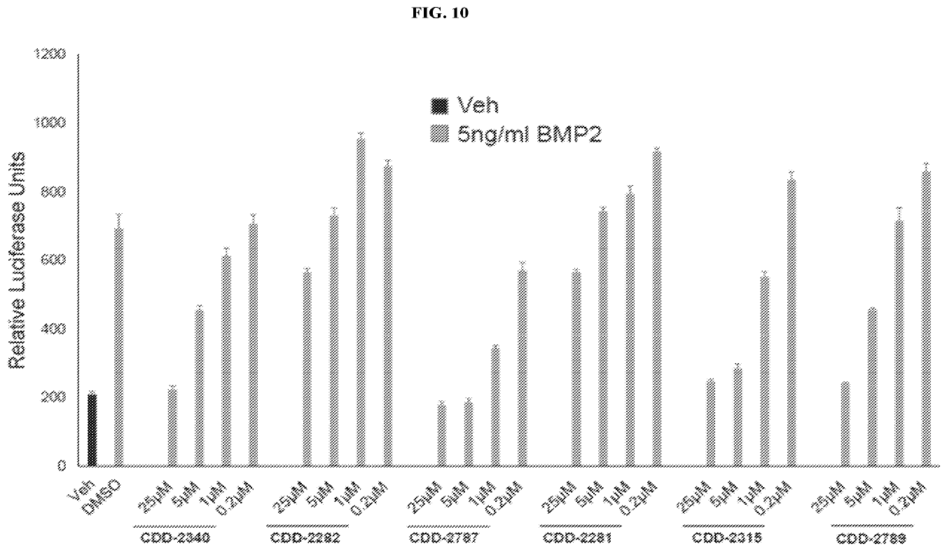

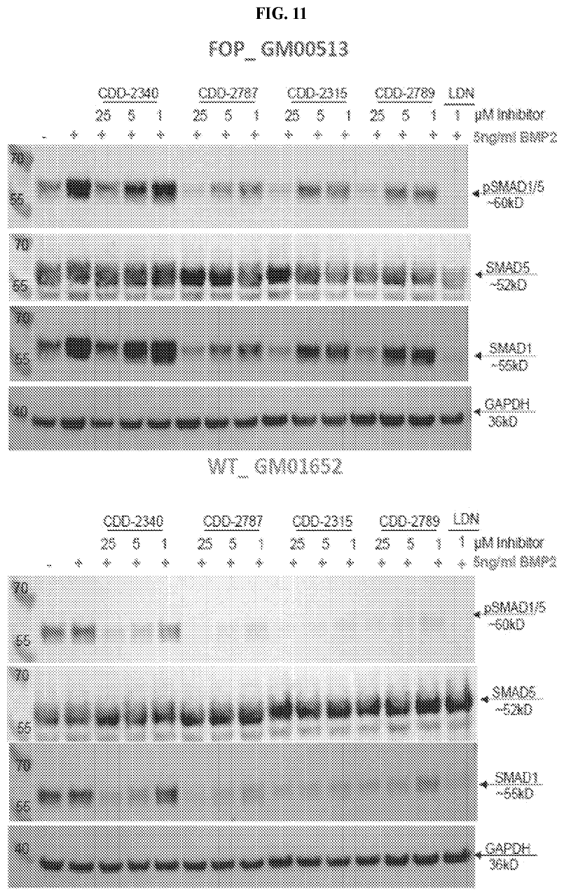

The present disclosure further provides exemplary pharmacokinetic data for compound CDD-2789. In the experiments described herein, mice were administered CDD-2789 (50 mg/kg) by oral gavage and the concentration of CDD-2789 was measured at 0, 0.83, 0.167, 0.25, 0.5, 1, 2, 4, 6, 8, and 24 h post-administration (FIG. 9). In certain embodiments, a half-life of about 3.42 h was observed for CDD-2789 in mice. Selected non-limiting exemplary noncompartmental pharmacokinetic parameters for CDD-2789 are provided herein (Table 4).

TABLE 4
Noncompartmental PK parameters for CDD-
2789 (n = 4, mean ± SD)

| Parameter | Unit | CDD-2789 |
|---|---|---|
| Tₘₐₓ | hr | 1.75 ± 0.50 |
| Cₘₐₓ | μmol/L | 4.59 ± 0.26 |
| T_{1/2} | hr | 3.42 ± 0.38 |
| AUC₀₋ₜ | hr*μmol/L | 32.16 ± 6.76 |
| AUC_{0-∞} | hr*μmol/L | 32.42 ± 6.76 |
| CL/F | mg(hr*μmol/L)/kg | 1.60 ± 0.36 |
| MRT₀₋₄ | hr | 4.60 ± 0.59 |
| MRT_{0-∞} | hr | 4.79 ± 0.64 |

The present disclosure further provides measurements of ALT and AST concentrations in the plasma of mice administered CDD-2789, as described elsewhere herein (Table 5).

TABLE 5
Plasma ALT and AST levels after CDD-2789
treatment (24 h post-administration)

| Mouse No. | Unit | ALT | AST |
|---|---|---|---|
| 1 | IU/L | 21.83 | 30.76 |
| 2 (not hemolyzed) | IU/L | 165.48 | 141.26 |
| 3 | IU/L | 25.81 | 42.08 |
| 4 | IU/L | 23.60 | 55.52 |
| Control-1 | IU/L | 34.56 | 41.81 |
| Control-2 | IU/L | 39.87 | 48.71 |

### Enumerated Embodiments

The following exemplary embodiments are provided, the numbering of which is not to be construed as designating levels of importance:
Embodiment 1 provides a compound of Formula (I), or a salt, solvate, stereoisomer, or isotopologue thereof:

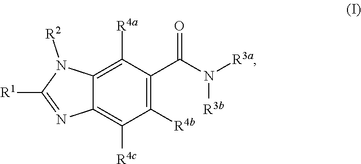

wherein:
R¹ is selected from the group consisting of optionally substituted C₆-C₁₀ aryl and optionally substituted C₂-C₁₂ heteroaryl;
R² is selected from the group consisting of H and optionally substituted C₃-C₈ cycloalkyl;
R^{3a} is selected from the group consisting of optionally substituted C₁-C₆ alkyl, optionally substituted C₇-C₁₃ aralkyl, and

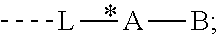

R^{3b} is H,
   or R^{3a} and R^{3b} can combine with the nitrogen to which they are bound to form an optionally substituted C₂-C₁₂ heterocycloalkyl;
R^{4a}, R^{4b}, and R^{4c} are each independently selected from the group consisting of H, halogen, CN, NO₂, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl;
L is selected from the group consisting of (optionally substituted C₁-C₆ alkylenyl)-*, -(optionally substituted C₃-C₆ cycloalkylenyl)-*, -(optionally substituted C₁-C₆ alkylenyl)C(═O)—*, -(optionally substituted C₃-C₆ cycloalkylenyl)C(═O)—*, —C(═O)(optionally substituted C₁-C₆ alkylenyl)-*, and —C(═O)(optionally substituted C₃-C₆ cycloalkylenyl)-*;
A is optionally substituted C₂-C₁₂ heterocyloalkylenyl;
B is selected from the group consisting of optionally substituted C₁-C₆ alkyl, optionally substituted C₃-C₁₂ cycloalkyl, optionally substituted C₇-C₁₃ aralkyl, optionally substituted C₆-C₁₀ aryl, optionally substituted C₂-C₁₂ heteroaryl, C(═O)(optionally substituted C₁-C₆ alkyl), C(═O)(optionally substituted C₃-C₁₂ cycloalkyl), C(═O)(optionally substituted C₇-C₁₃ aralkyl), C(═O)(optionally substituted C₆-C₁₀ aryl), and C(═O)(optionally substituted C₂-C₁₂ heteroaryl); and
*indicates a bond between L and A.
Embodiment 2 provides the compound of Embodiment 1, wherein R¹ is selected from the group consisting of:

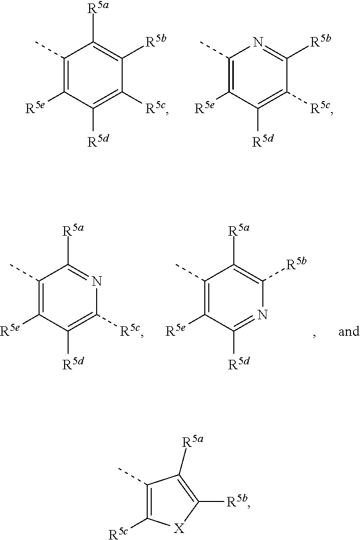

wherein:
R^{5a}, R^{5b}, R^{5c}, R^{5d}, and R^{e}, if present, are each independently selected from the group consisting of H, C(═O)R^{a}, C(═O)OR^{a}, C(═O)N(R^{a})(R^{b}), N(R^{a})(R^{b}), OR^{a}, halogen, optionally substituted C₁-C₆ alkyl, optionally substituted C₁-C₆ haloalkyl, optionally substituted C₆-C₁₀ aryl, optionally substituted C₂-C₁₂ heteroaryl,
   wherein any two vicinal substituents selected from the group consisting of R^{5a},
R^{5b}, R^{5c}, R^{5d}, and R^{5e} can combine with the atoms to which they are bound to form an optionally substituted C₆-C₁₀ aryl or optionally substituted C₂-C₁₂ heteroaryl;
R⁶ is selected from the group consisting of H, C(═O)OR^{a}, C(═O)N(R^{a})(R^{b}), optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl;
X, if present, is selected from the group consisting of N(R⁶), O, and S; and
R^{a} and R^{b} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.
Embodiment 3 provide the compound of Embodiment 2, wherein R^{5a}, R^{5b}, R^{5c}, R^{5d} and R^{5e}, if present, are each independently selected from the group consisting of H, OMe, C(═O)NH₂, F, I, phenyl, and 4-methoxyphenyl.
Embodiment 4 provides the compound of any one of Embodiments 1-3, wherein R¹ is selected from the group consisting of

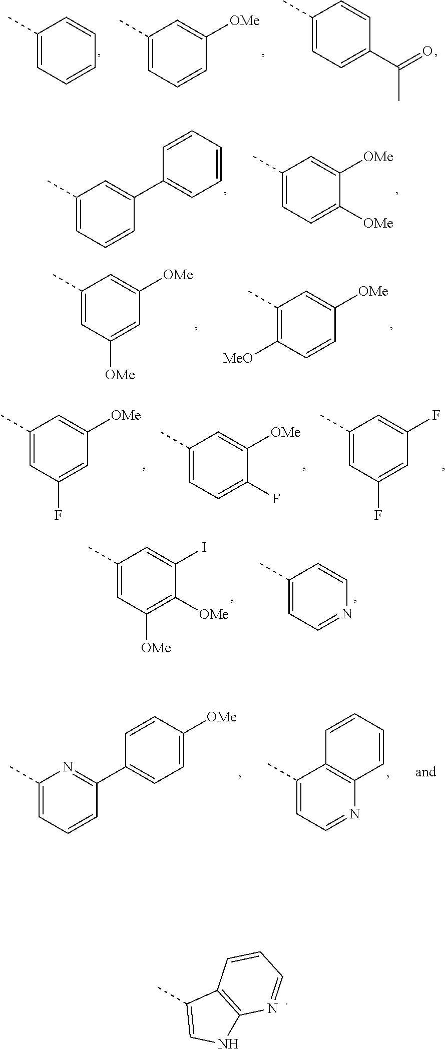

Embodiment 5 provides the compound of any one of Embodiments 1-4, wherein R² is selected from the group consisting of:

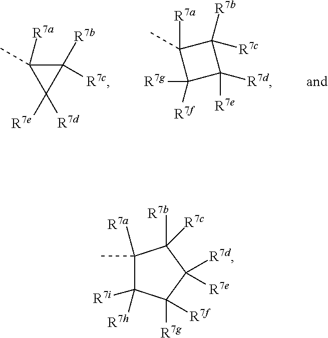

wherein:
R^{7a}, R^{7b}, R^{7c}, R^{7d}, R^{7e}, R^{7f}, R^{7g}, R^{7h}, and R⁷ⁱ, if present, are each independently selected from the group consisting of H, halogen, C(═O)R^{c}, C(═O)OR^{c}, C(═O)N(R^{c})(R^{d}), N(R^{c})(R^{d}), OR^{c}, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl; and
R^{c} and R^{d} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.
Embodiment 6 provides the compound of Embodiment 5, wherein at least one of the following applies:
(a) at least one selected from R^{7a}, R^{7b}, R^{7c}, R^{7d}, R^{7e}, R^{7f}, R^{7g}, R^{7h}, and R⁷ⁱ is H;
(b) at least two selected from R^{7a}, R^{7b}, R^{7c}, R^{7d}, R^{7e}, R^{7f}, R^{7g}, R^{7h}, and R⁷ⁱ are H;
(c) at least three selected from R^{7a}, R^{7b}, R^{7c}, R^{7d}, R^{7e}, R^{7f}, R^{7g}, R^{7h}, and R⁷ⁱ are H;
(d) at least four selected from R^{7a}, R^{7b}, R^{7c}, R^{7d}, R^{7e}, R^{7f}, R^{7g}, R^{7h}, and R⁷ⁱ are H;
(e) at least five selected from R^{7a}, R^{7b}, R^{7c}, R^{7d}, R^{7e}, R^{7f}, R^{7g}, R^{7h}, and RTi are H;
(f) at least six selected from R^{7a}, R^{7b}, RTe, R^{7d}, Rle, R^{7f}, R^{7g}, R^{7h}, and R⁷ⁱ are H;
(g) at least seven selected from R^{7a}, R^{7b}, R^{7c}, R^{7d}, R^{7e}, R^{7f}, R^{7g}, R^{7h}, and R⁷ⁱ are H;
(h) at least eight selected from R^{7a}, R^{7b}, R^{7c}, R^{7d}, R^{7e}, R^{7f}, R^{7g}, R^{7h}, and R⁷ⁱ are H; and
(i) each of R^{7a}, R^{7b}, R^{7c}, R^{7d}, R^{7e}, R^{7f}, R^{7g}, R^{7h}, and R⁷ⁱ are H.
Embodiment 7 provides the compound of any one of Embodiments 1-6, wherein R² is selected from the group consisting of
H,

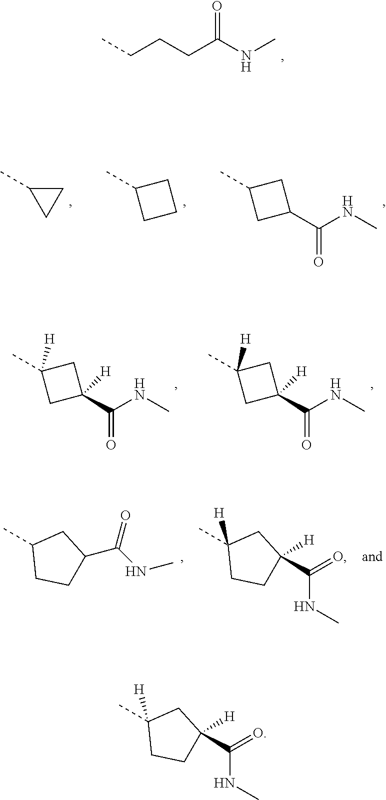

Embodiment 8 provides the compound of any one of Embodiments 1-7, wherein R^{3a} is

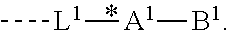

Embodiment 9 provides the compound of any one of Embodiments 1-8, wherein L is selected from the group consisting of —CH₂—*, —CH₂CH₂—*, —CH₂CH₂CH₂—*, —CH₂CH₂C(═O)—* and

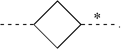

Embodiment 10 provides the compound of any one of Embodiments 1-9, wherein A is:

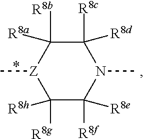

wherein:
R^{8a}, R^{8b}, R^{8c}, R^{8d}, R^{8e}, R^{8f}, R^{8g}, R^{8h}, and R⁸ⁱ, if present, are each independently selected from the group consisting of H, halogen, C(═O)R^{c}, C(═O)OR^{e}, C(═O)N(R^{e})(R^{f}), N(R^{e})(R^{f}), OR^{e}, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl,
   wherein two geminal substituents selected from the group consisting of R^{8a}, R^{8b}, R^{8c}, R^{8d}, R^{8e}, R^{8f}, R^{8g}, and R^{8h} can combine with the carbon atom to which they are bound to form a C(═O);
Z is selected from the group consisting of N and CR⁸ⁱ, and
R^{e} and R^{f} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl
Embodiment 11 provide the compound of Embodiment 10, wherein at least one of the following applies:
(a) at least one selected from R^{8a}, R^{8b}, R^{8c}, R^{8d}, R^{8e}, R^{8f}, R^{8g}, R^{8h}, and R⁸ⁱ is H;
(b) at least two selected from R^{8a}, R^{8b}, R^{8c}, R^{8d}, R^{8e}, R^{8f}, R^{8g}, R^{8h}, and R⁸ⁱ are H;
(c) at least three selected from R^{8a}, R^{8b}, R^{8c}, R^{8d}, R^{8e}, R^{8f}, R^{8g}, R^{8h}, and R⁸ⁱ are H;
(d) at least four selected from R^{8a}, R^{8b}, R^{8c}, R^{8d}, R^{8e}, R^{8f}, R^{8g}, R^{8h}, and R⁸ⁱ are H;
(e) at least five selected from R^{8a}, R^{8b}, R^{8c}, R^{8d}, R^{8e}, R^{8f}, R^{8g}, R^{8h}, and R⁸ⁱ are H;
(f) at least six selected from R^{8a}, R^{8b}, R^{8c}, R^{8d}, R^{8e}, R^{8f}, R^{8g}, R^{8h}, and R⁸ⁱ are H;
(g) at least seven selected from R^{8a}, R^{8b}, R^{8c}, R^{8d}, R^{8e}, R^{8f}, R^{8g}, R^{8h}, and R⁸ⁱ are H;
(h) at least eight selected from R^{8a}, R^{8b}, R^{8e}, R^{8d}, R^{8e}, R^{8f}, R^{8g}, R^{8h}, and R⁸ⁱ are H; and

(i) each of R^{8a}, R^{8b}, R^{8c}, R^{8d}, R^{8e}, R^{8f}, R^{8g}, R^{8h}, and R⁸ⁱ are H.
Embodiment 12 provides the compound of Embodiment 10 or 11, wherein at least one of the following applies:
(a) R^{8a}, R^{8b}, R^{8c}, R^{8d}, R^{8e}, R^{8f}, R^{8g}, R^{8h}, and R⁸ⁱ are each independently selected from the group consisting of H, OH, and phenyl; and
(b) at least one pair of geminal substituents selected from the group consisting of R^{8a} and R^{8b}, R^{8c} and R^{8d}, R^{8e} and R^{8f}, and R⁸⁸ and R^{8h} combine with the carbon atom to which they are bound to form a C(═O).
Embodiment 13 provides the compound of any one of Embodiments 1-12, wherein A is selected from the group consisting of

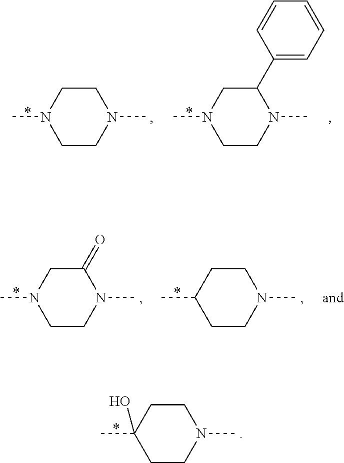

Embodiment 14 provides the compound of any one of Embodiments 1-13, wherein B is selected from the group consisting of
methyl,

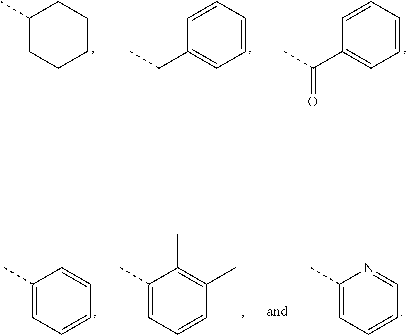

Embodiment 15 provides the compound of any one of Embodiments 1-4, wherein R^{3a}
   and R^{3b} combine to form

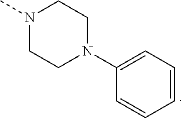

Embodiment 16 provides the compound of any one of Embodiments 1-14, wherein R^{3a} is selected from the group consisting of
   methyl,

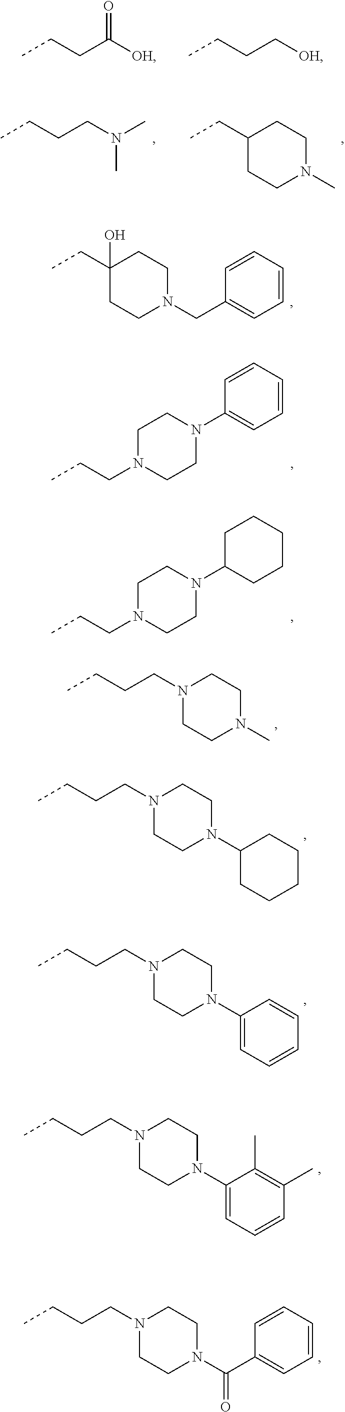

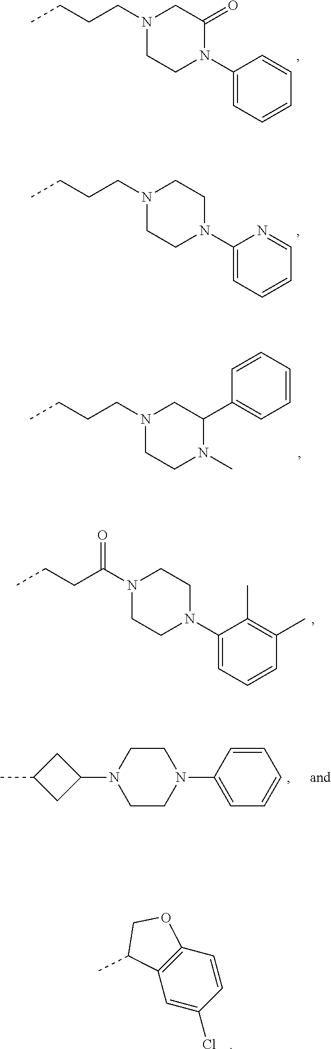

Embodiment 17 provides the compound of any one of Embodiments 1-16, wherein each occurrence of alkyl, cycloalkyl, alkylenyl, cycloalkylenyl, heterocycloalkylenyl, heterocycloalkyl, heteroalkyl, aralkyl, aryl, heteroaryl, is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂ OR^{I}, N(R^{I})(R^{II}), C₁-C₆ haloalkoxy, C₃-C₈ halocycloalkoxy, aryl, heteroaryl, (C₁-C₆ alkylenyl)C(═O)N(R^{I})(R^{II}), (C₁-C₆ alkylenyl)C(═O)OR^{I}, O(C₁-C₃ alkylenyl)C(═O)OR^{II}, O(C₁-C₃ alkylenyl)C(═O)N(R^{I})(R^{II}), C(═O)R^{I}, C(═O)OR^{I}, OC(═O)R^{I}, OC(═O)OR^{I}, SR^{I}, S(═O)R^{I}, S(═O)₂R^{I}, S(═O)₂N(R^{I})(R^{I}), S(═O)₂NR^{I}C(═O)NHR^{II}, N(R^{I})S(═O)₂R^{II}, N(R^{I})C(═O)R^{II}, and C(═O)NR^{I}R^{II}, wherein R^{I} and R^{II} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.
Embodiment 18 provides the compound of any one of Embodiments 1-17, which is selected from the group consisting of:
2-(3-iodo-4,5-dimethoxyphenyl)-1-((1S,3S)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-((1R,3S)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-((1S,3R)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-((1R,3R)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-(3-(methylcarbamoyl)cyclopentyl)-N-(3-(4-phenylpiperazin-1-yl)propyl)-1H-benzo[d]imidazole-6-carboxamide;
1-((1S,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1R,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1S,3R)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1R,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-(3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
N-(5-chloro-2,3-dihydrobenzofuran-3-yl)-1-((1S,3S)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-((1R,3S)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-((1S,3R)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-((1R,3R)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-(3-(methylcarbamoyl)cyclopentyl)-2-(3,4,5-trimethoxyphenyl)-1H-benzo[d]imidazole-6-carboxamide;
N-methyl-1-((1S,3S)-3-(methylcarbamoyl)cyclopentyl)-2-(3,4,5-trimethoxyphenyl)-1H-benzo[d]imidazole-6-carboxamide;
N-methyl-1-((1R,3S)-3-(methylcarbamoyl)cyclopentyl)-2-(3,4,5-trimethoxyphenyl)-1H-benzo[d]imidazole-6-carboxamide;
N-methyl-1-((1S,3R)-3-(methylcarbamoyl)cyclopentyl)-2-(3,4,5-trimethoxyphenyl)-1H-benzo[d]imidazole-6-carboxamide;
N-methyl-1-((1R,3R)-3-(methylcarbamoyl)cyclopentyl)-2-(3,4,5-trimethoxyphenyl)-1H-benzo[d]imidazole-6-carboxamide;
N-methyl-1-(3-(methylcarbamoyl)cyclopentyl)-2-(3,4,5-trimethoxyphenyl)-1H-benzo[d]imidazole-6-carboxamide;
1-((1r,3r)-3-(methylcarbamoyl)cyclobutyl)-N-(3-(4-phenylpiperazin-1-yl)propyl)-2-(3,4,5-trimethoxyphenyl)-1H-benzo[d]imidazole-6-carboxamide;
1-((1r,3s)-3-(methylcarbamoyl)cyclobutyl)-N-(3-(4-phenylpiperazin-1-yl)propyl)-2-(3,4,5-trimethoxyphenyl)-1H-benzo[d]imidazole-6-carboxamide;
1-(3-(methylcarbamoyl)cyclobutyl)-N-(3-(4-phenylpiperazin-1-yl)propyl)-2-(3,4,5-trimethoxyphenyl)-1H-benzo[d]imidazole-6-carboxamide;
N-(3-(4-phenylpiperazin-1-yl)propyl)-2-(3,4,5-trimethoxyphenyl)-1H-benzo[d]imidazole-6-carboxamide;
(1r,3r)-N-methyl-3-(6-(4-phenylpiperazine-1-carbonyl)-2-(3,4,5-trimethoxyphenyl)-1H-benzo[d]imidazol-1-yl)cyclobutane-1-carboxamide;
(1r,3s)-N-methyl-3-(6-(4-phenylpiperazine-1-carbonyl)-2-(3,4,5-trimethoxyphenyl)-1H-benzo[d]imidazol-1-yl)cyclobutane-1-carboxamide;
N-methyl-3-(6-(4-phenylpiperazine-1-carbonyl)-2-(3,4,5-trimethoxyphenyl)-1H-benzo[d]imidazol-1-yl)cyclobutane-1-carboxamide;
1-((1r,3r)-3-(methylcarbamoyl)cyclobutyl)-N-(3-(4-methylpiperazin-1-yl)propyl)-2-(3,4,5-trimethoxyphenyl)-1H-benzo[d]imidazole-6-carboxamide;
1-((1r,3s)-3-(methylcarbamoyl)cyclobutyl)-N-(3-(4-methylpiperazin-1-yl)propyl)-2-(3,4,5-trimethoxyphenyl)-1H-benzo[d]imidazole-6-carboxamide;
1-(3-(methylcarbamoyl)cyclobutyl)-N-(3-(4-methylpiperazin-1-yl)propyl)-2-(3,4,5-trimethoxyphenyl)-1H-benzo[d]imidazole-6-carboxamide;
N-(3-(dimethylamino)propyl)-1-((1r,3r)-3-(methylcarbamoyl)cyclobutyl)-2-(3,4,5-trimethoxyphenyl)-1H-benzo[d]imidazole-6-carboxamide;
N-(3-(dimethylamino)propyl)-1-((1r,3s)-3-(methylcarbamoyl)cyclobutyl)-2-(3,4,5-trimethoxyphenyl)-1H-benzo[d]imidazole-6-carboxamide;
N-(3-(dimethylamino)propyl)-1-(3-(methylcarbamoyl)cyclobutyl)-2-(3,4,5-trimethoxyphenyl)-1H-benzo[d]imidazole-6-carboxamide;
1-((1r,3r)-3-(methylcarbamoyl)cyclobutyl)-N-((1-methylpiperidin-4-yl)methyl)-2-(3,4,5-trimethoxyphenyl)-1H-benzo[d]imidazole-6-carboxamide;
1-((1r,3s)-3-(methylcarbamoyl)cyclobutyl)-N-((1-methylpiperidin-4-yl)methyl)-2-(3,4,5-trimethoxyphenyl)-1H-benzo[d]imidazole-6-carboxamide;
1-(3-(methylcarbamoyl)cyclobutyl)-N-((1-methylpiperidin-4-yl)methyl)-2-(3,4,5-trimethoxyphenyl)-1H-benzo[d]imidazole-6-carboxamide;
2-(3-methoxyphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(3-methoxyphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(3-methoxyphenyl)-1-(3-(methylcarbamoyl)cyclobutyl)-N-(3-(4-phenylpiperazin-1-yl)propyl)-1H-benzo[d]imidazole-6-carboxamide;
2-(3,5-dimethoxyphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(3,5-dimethoxyphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(3,5-dimethoxyphenyl)-1-(3-(methylcarbamoyl)cyclobutyl)-N-(3-(4-phenylpiperazin-1-yl)propyl)-1H-benzo[d]imidazole-6-carboxamide;
1-cyclobutyl-N-(3-(4-phenylpiperazin-1-yl)propyl)-2-(3,4,5-trimethoxyphenyl)-1H-benzo[d]imidazole-6-carboxamide;
1-(4-(methylamino)-4-oxobutyl)-N-(3-(4-phenylpiperazin-1-yl)propyl)-2-(3,4,5-trimethoxyphenyl)-1H-benzo[d]imidazole-6-carboxamide;
1-((1r,3r)-3-(methylcarbamoyl)cyclobutyl)-2-phenyl-N-(3-(4-phenylpiperazin-1-yl)propyl)-1H-benzo[d]imidazole-6-carboxamide;
1-((1r,3s)-3-(methylcarbamoyl)cyclobutyl)-2-phenyl-N-(3-(4-phenylpiperazin-1-yl)propyl)-1H-benzo[d]imidazole-6-carboxamide;
1-(3-(methylcarbamoyl)cyclobutyl)-2-phenyl-N-(3-(4-phenylpiperazin-1-yl)propyl)-1H-benzo[d]imidazole-6-carboxamide;
2-(4-carbamoylphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(4-carbamoylphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(4-carbamoylphenyl)-1-(3-(methylcarbamoyl)cyclobutyl)-N-(3-(4-phenylpiperazin-1-yl)propyl)-1H-benzo[d]imidazole-6-carboxamide;
2-(3-fluoro-5-methoxyphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(3-fluoro-5-methoxyphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(3-fluoro-5-methoxyphenyl)-1-(3-(methylcarbamoyl)cyclobutyl)-N-(3-(4-phenylpiperazin-1-yl)propyl)-1H-benzo[d]imidazole-6-carboxamide;
N-(3-(4-(2,3-dimethylphenyl)piperazin-1-yl)propyl)-1-((1r,3r)-3-(methylcarbamoyl)cyclobutyl)-2-(3,4,5-trimethoxyphenyl)-1H-benzo[d]imidazole-6-carboxamide;
N-(3-(4-(2,3-dimethylphenyl)piperazin-1-yl)propyl)-1-((1r,3s)-3-(methylcarbamoyl)cyclobutyl)-2-(3,4,5-trimethoxyphenyl)-1H-benzo[d]imidazole-6-carboxamide;
N-(3-(4-(2,3-dimethylphenyl)piperazin-1-yl)propyl)-1-(3-(methylcarbamoyl)cyclobutyl)-2-(3,4,5-trimethoxyphenyl)-1H-benzo[d]imidazole-6-carboxamide;
1-cyclobutyl-N-(3-(4-(2,3-dimethylphenyl)piperazin-1-yl)propyl)-2-(3,4,5-trimethoxyphenyl)-1H-benzo[d]imidazole-6-carboxamide;
2-(2,5-dimethoxyphenyl)-N-(3-(4-(2,3-dimethylphenyl)piperazin-1-yl)propyl)-1-((1r,3r)-3-(methylcarbamoyl)cyclobutyl)-1H-benzo[d]imidazole-6-carboxamide;
2-(2,5-dimethoxyphenyl)-N-(3-(4-(2,3-dimethylphenyl)piperazin-1-yl)propyl)-1-((1r,3s)-3-(methylcarbamoyl)cyclobutyl)-1H-benzo[d]imidazole-6-carboxamide;
2-(2,5-dimethoxyphenyl)-N-(3-(4-(2,3-dimethylphenyl)piperazin-1-yl)propyl)-1-(3-(methylcarbamoyl)cyclobutyl)-1H-benzo[d]imidazole-6-carboxamide;
2-(3,4-dimethoxyphenyl)-N-(3-(4-(2,3-dimethylphenyl)piperazin-1-yl)propyl)-1-((1r,3r)-3-(methylcarbamoyl)cyclobutyl)-1H-benzo[d]imidazole-6-carboxamide;
2-(3,4-dimethoxyphenyl)-N-(3-(4-(2,3-dimethylphenyl)piperazin-1-yl)propyl)-1-((1r,3s)-3-(methylcarbamoyl)cyclobutyl)-1H-benzo[d]imidazole-6-carboxamide;
2-(3,4-dimethoxyphenyl)-N-(3-(4-(2,3-dimethylphenyl)piperazin-1-yl)propyl)-1-(3-(methylcarbamoyl)cyclobutyl)-1H-benzo[d]imidazole-6-carboxamide;
3-(1-cyclobutyl-2-(3,4,5-trimethoxyphenyl)-1H-benzo[d]imidazole-6-carboxamido)propanoic acid;
1-cyclobutyl-N-(3-hydroxypropyl)-2-(3,4,5-trimethoxyphenyl)-1H-benzo[d]imidazole-6-carboxamide;
1-cyclobutyl-N-(3-(4-(2,3-dimethylphenyl)piperazin-1-yl)-3-oxopropyl)-2-(3,4,5-trimethoxyphenyl)-1H-benzo[d]imidazole-6-carboxamide;
1-cyclopropyl-N-(3-(4-(2,3-dimethylphenyl)piperazin-1-yl)propyl)-2-(3,4,5-trimethoxyphenyl)-1H-benzo[d]imidazole-6-carboxamide;
1-cyclobutyl-2-(3,5-dimethoxyphenyl)-N-(3-(4-(2,3-dimethylphenyl)piperazin-1-yl)propyl)-1H-benzo[d]imidazole-6-carboxamide;
1-cyclobutyl-N-(3-(dimethylamino)propyl)-2-(3-fluoro-5-methoxyphenyl)-1H-benzo[d]imidazole-6-carboxamide;
1-cyclobutyl-2-(3,5-difluorophenyl)-N-(3-(dimethylamino)propyl)-1H-benzo[d]imidazole-6-carboxamide;
1-cyclobutyl-N-(3-(dimethylamino)propyl)-2-(pyridin-4-yl)-1H-benzo[d]imidazole-6-carboxamide;
1-cyclobutyl-N-(3-(dimethylamino)propyl)-2-(6-(4-methoxyphenyl)pyridin-2-yl)-1H-benzo[d]imidazole-6-carboxamide;
2-([1,1′-biphenyl]-3-yl)-1-cyclobutyl-N-(3-(dimethylamino)propyl)-1H-benzo[d]imidazole-6-carboxamide;
1-((1R,3R)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1S,3R)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1R,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1S,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-(3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-cyclobutyl-N-(3-(dimethylamino)propyl)-2-(isoquinolin-4-yl)-1H-benzo[d]imidazole-6-carboxamide;
1-cyclobutyl-N-(3-(dimethylamino)propyl)-2-(quinolin-4-yl)-1H-benzo[d]imidazole-6-carboxamide;
1-cyclobutyl-N-(3-(dimethylamino)propyl)-2-(1H-pyrrolo[2,3-b]pyridin-3-yl)-1H-benzo[d]imidazole-6-carboxamide;
N-(3-(dimethylamino)propyl)-2-(3-fluoro-5-methoxyphenyl)-1-((1r,3r)-3-(methylcarbamoyl)cyclobutyl)-1H-benzo[d]imidazole-6-carboxamide;
N-(3-(dimethylamino)propyl)-2-(3-fluoro-5-methoxyphenyl)-1-((1r,3s)-3-(methylcarbamoyl)cyclobutyl)-1H-benzo[d]imidazole-6-carboxamide;
N-(3-(dimethylamino)propyl)-2-(3-fluoro-5-methoxyphenyl)-1-(3-(methylcarbamoyl)cyclobutyl)-1H-benzo[d]imidazole-6-carboxamide;
N-(3-(dimethylamino)propyl)-2-(4-fluoro-3-methoxyphenyl)-1-((1r,3r)-3-(methylcarbamoyl)cyclobutyl)-1H-benzo[d]imidazole-6-carboxamide;
N-(3-(dimethylamino)propyl)-2-(4-fluoro-3-methoxyphenyl)-1-((1r,3s)-3-(methylcarbamoyl)cyclobutyl)-1H-benzo[d]imidazole-6-carboxamide;
N-(3-(dimethylamino)propyl)-2-(4-fluoro-3-methoxyphenyl)-1-(3-(methylcarbamoyl)cyclobutyl)-1H-benzo[d]imidazole-6-carboxamide;
N-(3-(dimethylamino)propyl)-2-(3-methoxyphenyl)-1-((1r,3r)-3-(methylcarbamoyl)cyclobutyl)-1H-benzo[d]imidazole-6-carboxamide;
N-(3-(dimethylamino)propyl)-2-(3-methoxyphenyl)-1-((1r,3s)-3-(methylcarbamoyl)cyclobutyl)-1H-benzo[d]imidazole-6-carboxamide;
N-(3-(dimethylamino)propyl)-2-(3-methoxyphenyl)-1-(3-(methylcarbamoyl)cyclobutyl)-1H-benzo[d]imidazole-6-carboxamide;
2-(3,4-dimethoxyphenyl)-N-(3-(dimethylamino)propyl)-1-((1r,3r)-3-(methylcarbamoyl)cyclobutyl)-1H-benzo[d]imidazole-6-carboxamide;
2-(3,4-dimethoxyphenyl)-N-(3-(dimethylamino)propyl)-1-((1r,3s)-3-(methylcarbamoyl)cyclobutyl)-1H-benzo[d]imidazole-6-carboxamide;
2-(3,4-dimethoxyphenyl)-N-(3-(dimethylamino)propyl)-1-(3-(methylcarbamoyl)cyclobutyl)-1H-benzo[d]imidazole-6-carboxamide;
2-(3,5-dimethoxyphenyl)-N-(3-(dimethylamino)propyl)-1-((1r,3r)-3-(methylcarbamoyl)cyclobutyl)-1H-benzo[d]imidazole-6-carboxamide;
2-(3,5-dimethoxyphenyl)-N-(3-(dimethylamino)propyl)-1-((1r,3s)-3-(methylcarbamoyl)cyclobutyl)-1H-benzo[d]imidazole-6-carboxamide;
2-(3,5-dimethoxyphenyl)-N-(3-(dimethylamino)propyl)-1-(3-(methylcarbamoyl)cyclobutyl)-1H-benzo[d]imidazole-6-carboxamide;
2-(2,5-dimethoxyphenyl)-N-(3-(dimethylamino)propyl)-1-((1r,3r)-3-(methylcarbamoyl)cyclobutyl)-1H-benzo[d]imidazole-6-carboxamide;
2-(2,5-dimethoxyphenyl)-N-(3-(dimethylamino)propyl)-1-((1r,3s)-3-(methylcarbamoyl)cyclobutyl)-1H-benzo[d]imidazole-6-carboxamide;
2-(2,5-dimethoxyphenyl)-N-(3-(dimethylamino)propyl)-1-(3-(methylcarbamoyl)cyclobutyl)-1H-benzo[d]imidazole-6-carboxamide;
1-cyclobutyl-N-(2-(4-phenylpiperazin-1-yl)ethyl)-2-(3,4,5-trimethoxyphenyl)-1H-benzo[d]imidazole-6-carboxamide;
1-cyclobutyl-N-(3-(4-(pyridin-2-yl)piperazin-1-yl)propyl)-2-(3,4,5-trimethoxyphenyl)-1H-benzo[d]imidazole-6-carboxamide;
1-cyclobutyl-N-(3-(4-phenylpiperazin-1-yl)cyclobutyl)-2-(3,4,5-trimethoxyphenyl)-1H-benzo[d]imidazole-6-carboxamide;
1-cyclobutyl-N-(3-(dimethylamino)propyl)-2-(3,4,5-trimethoxyphenyl)-1H-benzo[d]imidazole-6-carboxamide;
1-cyclobutyl-N-(3-(3-oxo-4-phenylpiperazin-1-yl)propyl)-2-(3,4,5-trimethoxyphenyl)-1H-benzo[d]imidazole-6-carboxamide;
N-(3-(4-benzoylpiperazin-1-yl)propyl)-1-cyclobutyl-2-(3,4,5-trimethoxyphenyl)-1H-benzo[d]imidazole-6-carboxamide;
1-cyclobutyl-N-(3-(4-cyclohexylpiperazin-1-yl)propyl)-2-(3,4,5-trimethoxyphenyl)-1H-benzo[d]imidazole-6-carboxamide;
1-cyclobutyl-N-(3-(4-methyl-3-phenylpiperazin-1-yl)propyl)-2-(3,4,5-trimethoxyphenyl)-1H-benzo[d]imidazole-6-carboxamide;
1-cyclobutyl-N-(2-(4-cyclohexylpiperazin-1-yl)ethyl)-2-(3,4,5-trimethoxyphenyl)-1H-benzo[d]imidazole-6-carboxamide; and
N-((1-benzyl-4-hydroxypiperidin-4-yl)methyl)-1-cyclobutyl-2-(3,4,5-trimethoxyphenyl)-1H-benzo[d]imidazole-6-carboxamide.
   Embodiment 19 provides an antibody-drug conjugate (ADC) comprising at least one compound of any one of Embodiments 1-18 and an antibody, wherein the compound is covalently conjugated to the antibody.
   Embodiment 20 provides the ADC of Embodiment 19, wherein the antibody specifically binds to AMHR2.
   Embodiment 21 provides the ADC of Embodiment 19 or 20, wherein the ADC is suitable for delivery to at least one cell type selected from the group consisting of ovarian granulosa cells and endometrioma cells.
   Embodiment 22 provides a pharmaceutical composition comprising at least one compound of any one of Embodiments 1-18 or the ADC of any one of Embodiments 19-21 and a pharmaceutically acceptable carrier.
   Embodiment 23 provides the pharmaceutical composition of Embodiment 22, further comprising at least one additional therapeutically effective agent.
   Embodiment 24 provides a method of treating, preventing, and/or ameliorating a disease or disorder in a subject in need thereof, the method comprising administering to the subject a therapeutically effective amount of at least one compound of any one of Embodiments 1-18, the ADC of any one of Embodiments 19-21, and/or the pharmaceutical composition of Embodiment 22 or 23.
   Embodiment 25 provides the method of Embodiment 24, wherein the disease or disorder is characterized by one or more ACVR1 activating mutations or overactive ACVR1.
   Embodiment 26 provides the method of Embodiment 24 or 25, wherein the disease or disorder is at least one selected from the group consisting of fibrodysplasia ossificans progressiva (FOP), atherosclerosis, anemia, iron deficiency, tumor angiogenesis and/or vasculogenesis, and cancer.
   Embodiment 27 provides the method of Embodiment 26, wherein the cancer is at least one selected from the group consisting of diffuse intrinsic pontine glioma (DIPG), ependymoma, breast cancer, endometrial cancer, and pancreatic cancer.
   Embodiment 28 provides the method of any one of Embodiments 24-27, wherein the subject is further administered at least one additional therapeutic agent.
   Embodiment 29 provides a method of inhibiting and/or reducing activity of ACVR1 in a subject in need thereof, the method comprising administering to the subject a therapeutically effective amount of at least one compound of any one of Embodiments 1-18, the ADC of any one of Embodiments 19-21, and/or the pharmaceutical composition of Embodiment 22 or 23.
   Embodiment 30 provides the method of Embodiment 29, wherein the ACVR1 is ACVR1^{WT} or an ACVR1 mutant comprising one or more activating mutations.
   Embodiment 31 provides a method of inhibiting and/or reducing activity of ACVRL1 in a subject in need thereof, the method comprising administering to the subject a therapeutically effective amount of at least one compound of any one of Embodiments 1-18, the ADC of any one of Embodiments 19-21, and/or the pharmaceutical composition of Embodiment 22 or 23.
   Embodiment 32 provides the method of Embodiment 31, wherein the ACVRL1 is ACVRL1^{WT} or an ACVRL1 mutant comprising one or more activating mutations.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound of Formula (I), or a salt, solvate, stereoisomer, or isotopologue thereof: 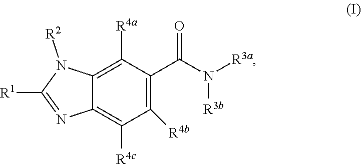
wherein:
R¹ is selected from the group consisting of optionally substituted C₆-C₁₀ aryl and optionally substituted C₂-C₁₂ heteroaryl;
R² is selected from the group consisting of H and optionally substituted C₃-C₈ cycloalkyl;
R^{3a} is selected from the group consisting of optionally substituted C₁-C₆ alkyl, optionally substituted C₇-C₁₃ aralkyl, and 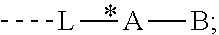
R^{3b} is H,
or R^{3a} and R^{3b} can combine with the nitrogen to which they are bound to form an optionally substituted C₂-C₁₂ heterocycloalkyl;
R^{4a}, R^{4b}, and R^{4c} are each independently selected from the group consisting of H, halogen, CN, NO₂, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl;
L is selected from the group consisting of (optionally substituted C₁-C₆ alkylenyl)-*, -(optionally substituted C₃-C₆ cycloalkylenyl)-*, -(optionally substituted C₁-C₆ alkylenyl)C(═O)—*, -(optionally substituted C₃-C₆ cycloalkylenyl)C(═O)—*, —C(═O)(optionally substituted C₁-C₆ alkylenyl)-*, and —C(═O)(optionally substituted C₃-C₆ cycloalkylenyl)-*;
A is optionally substituted C₂-C₁₂ heterocyloalkylenyl;
B is selected from the group consisting of optionally substituted C₁-C₆ alkyl, optionally substituted C₃-C₁₂ cycloalkyl, optionally substituted C₇-C₁₃ aralkyl, optionally substituted C₆-C₁₀ aryl, optionally substituted C₂-C₁₂ heteroaryl, C(═O)(optionally substituted C₁-C₆ alkyl), C(═O)(optionally substituted C₃-C₁₂ cycloalkyl), C(═O)(optionally substituted C₇-C₁₃ aralkyl), C(═O)(optionally substituted C₆-C₁₀ aryl), and C(═O)(optionally substituted C₂-C₁₂ heteroaryl); and
* indicates a bond between L and A.

2. The compound of claim 1, wherein R¹ is selected from the group consisting of: 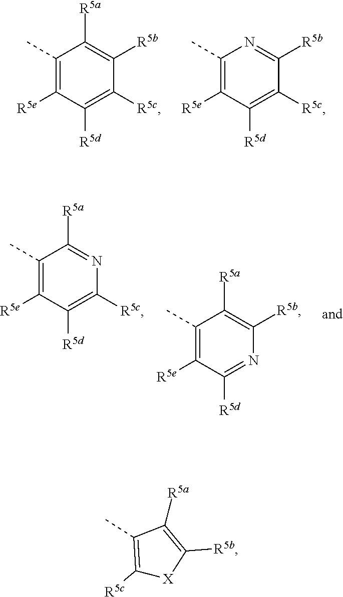
wherein:
R^{5a}, R^{5b}, R^{5c}, R^{5d}, and R^{5e}, if present, are each independently selected from the group consisting of H, C(═O)R^{a}, C(═O)OR^{a}, C(═O)N(R^{a})(R^{b}), N(R^{a})(R^{b}), OR^{a}, halogen, optionally substituted C₁-C₆ alkyl, optionally substituted C₁-C₆ haloalkyl, optionally substituted C₆-C₁₀ aryl, optionally substituted C₂-C₁₂ heteroaryl,
wherein any two vicinal substituents selected from the group consisting of R^{5a}, R^{5b}, R^{5c}, R^{5d}, and R^{5e} can combine with the atoms to which they are bound to form an optionally substituted C₆-C₁₀ aryl or optionally substituted C₂-C₁₂ heteroaryl;
R⁶ is selected from the group consisting of H, C(═O)OR^{a}, C(═O)N(R^{a})(R^{b}), optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl;
X, if present, is selected from the group consisting of N(R⁶), O, and S; and
R^{a} and R^{b} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.

3. The compound of claim 2, wherein R^{5a}, R^{5b}, R^{5c}, R^{5d}, and R^{5e}, if present, are each independently selected from the group consisting of H, OMe, C(═O)NH₂, F, I, phenyl, and 4-methoxyphenyl.

4. The compound of claim 1, wherein at least one of the following applies:
(a) R¹ is selected from the group consisting of 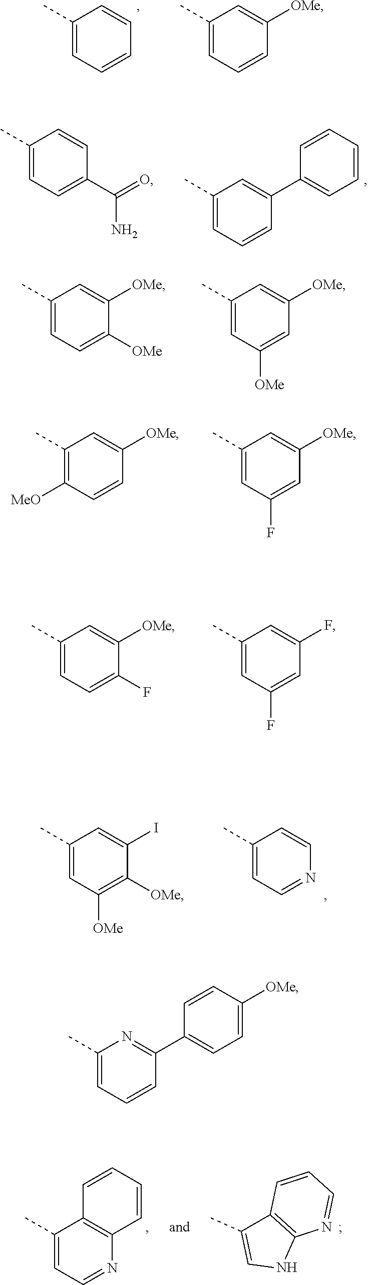
(b) R^{3a} is 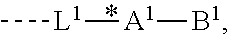
or R^{3a} and R^{3b} combine to form 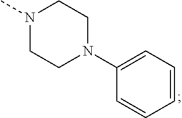
and
(c) R² is selected from the group consisting of
H,

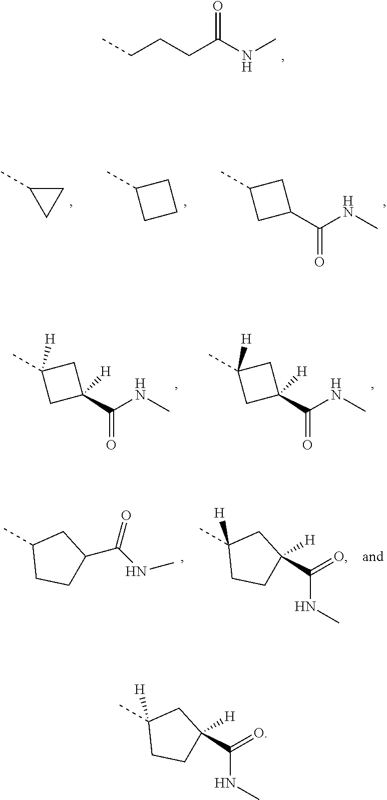

5. The compound of claim 1, wherein R² is selected from the group consisting of: 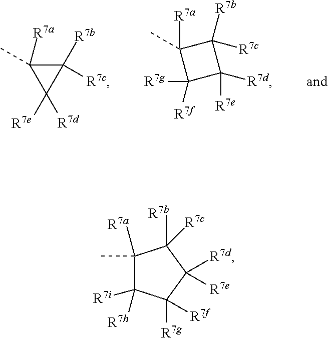
wherein:
R^{7a}, R^{7b}, R^{7c}, R^{7d}, R^{7e}, R^{7f}, R^{7g}, R^{7h}, and R⁷ⁱ, if present, are each independently selected from the group consisting of H, halogen, C(═O)R^{c}, C(═O)OR^{c}, C(═O)N(R^{c})(R^{d}), N(R^{c})(R^{d}), OR^{c}, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl; and
R^{c} and R^{d} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.

6. The compound of claim 5, wherein at least one of the following applies:
(a) at least one selected from R^{7a}, R^{7b}, R^{7c}, R^{7d}, R^{7e}, R^{7f}, R^{7g}, R^{7h}, and R⁷ⁱ is H;
(b) at least two selected from R^{7a}, R^{7b}, R^{7c}, R^{7d}, R^{7e}, R^{7f}, R^{7g}, R^{7h}, and R⁷ⁱ are H;
(c) at least three selected from R^{7a}, R^{7b}, R^{7c}, R^{7d}, R^{7e}, R^{7f}, R^{7g}, R^{7h}, and R⁷ⁱ are H;
(d) at least four selected from R^{7a}, R^{7b}, R^{7c}, R^{7d}, R^{7e}, R^{7f}, R^{7g}, R^{7h}, and R⁷ⁱ are H;
(e) at least five selected from R^{7a}, R^{7b}, R^{7c}, R^{7d}, R^{7e}, R^{7f}, R^{7g}, R^{7h}, and R⁷ⁱ are H;
(f) at least six selected from R^{7a}, R^{7b}, R^{7c}, R^{7d}, R^{7e}, R^{7f}, R^{7g}, R^{7h}, and R⁷ⁱ are H;
(g) at least seven selected from R^{7a}, R^{7b}, R^{7c}, R^{7d}, R^{7e}, R^{7f}, R^{7g}, R^{7h}, and R⁷ⁱ are H;
(h) at least eight selected from R^{7a}, R^{7b}, R^{7c}, R^{7d}, R^{7e}, R^{7f}, R^{7g}, R^{7h}, and R⁷ⁱ are H; and
(i) each of R^{7a}, R^{7b}, R^{7c}, R^{7d}, R^{7e}, R^{7f}, R^{7g}, R^{7h}, and R⁷ⁱ are H.

7-9. (canceled)

10. The compound of claim 1, wherein A is: 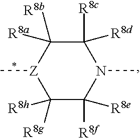
wherein:
R^{8a}, R^{8b}, R^{8c}, R^{8d}, R^{8e}, R^{8f}, R^{8g}, R^{8h}, and R⁸ⁱ, if present, are each independently selected from the group consisting of H, halogen, C(═O)R^{e}, C(═O)OR^{e}, C(═O)N(R^{e})(R^{f}), N(R^{e})(R^{f}), OR^{e}, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl,
wherein two geminal substituents selected from the group consisting of R^{8a}, R^{8b}, R^{8c}, R^{8d}, R^{8e}, R^{8f}, R^{8g}, and R^{8h} can combine with the carbon atom to which they are bound to form a C(═O);
Z is selected from the group consisting of N and CR⁸ⁱ, and
R^{e} and R^{f} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.

11. The compound of claim 10, wherein at least one of the following applies:
(a) at least one selected from R^{8a}, R^{8b}, R^{8c}, R^{8d}, R^{8e}, R^{8f}, R^{8g}, R^{8h}, and R⁸ⁱ is H;
(b) at least two selected from R^{8a}, R^{8b}, R^{8c}, R^{8d}, R^{8e}, R^{8f}, R^{8g}, R^{8h}, and R⁸ⁱ are H;
(a) at least three selected from R^{8a}, R^{8b}, R^{8c}, R^{8d}, R^{8e}, R^{8f}, R^{8g}, R^{8h}, and R⁸ⁱ are H;
(d) at least four selected from R^{8a}, R^{8b}, R^{8c}, R^{8d}, R^{8e}, R^{8f}, R^{8g}, R^{8h}, and R⁸ⁱ are H;
(e) at least five selected from R^{8a}, R^{8b}, R^{8c}, R^{8d}, R^{8e}, R^{8f}, R^{8g}, R^{8h}, and R⁸ⁱ are H;
(f) at least six selected from R^{8a}, R^{8b}, R^{8c}, R^{8d}, R^{8e}, R^{8f}, R^{8g}, R^{8h}, and R⁸ⁱ are H;
(g) at least seven selected from R^{8a}, R^{8b}, R^{8c}, R^{8d}, R^{8e}, R^{8f}, R^{8g}, R^{8h}, and R⁸ⁱ are H;
(h) at least eight selected from R^{8a}, R^{8b}, R^{8c}, R^{8d}, R^{8e}, R^{8f}, R^{8g}, R^{8h}, and R⁸ⁱ are H; and
(i) each of R^{8a}, R^{8b}, R^{8c}, R^{8d}, R^{8e}, R^{8f}, R^{8g}, R^{8h}, and R⁸ⁱ are H.

12. The compound of claim 10, wherein at least one of the following applies:
(a) R^{8a}, R^{8b}, R^{8c}, R^{8d}, R^{8e}, R^{8f}, R^{8g}, R^{8h}, and R⁸ⁱ are each independently selected from the group consisting of H, OH, and phenyl; and
(b) at least one pair of geminal substituents selected from the group consisting of R^{8a} and R^{8b}, R^{8c} and R^{8d}, R^{8e} and R^{8f}, and R^{8g} and R^{8h} combine with the carbon atom to which they are bound to form a C(═O).

13. The compound of claim 1, wherein at least one of the following applies:
(a) L is selected from the group consisting of —CH₂—*, —CH₂CH₂—*, —CH₂CH₂CH₂—*, —CH₂CH₂C(═O)—* and 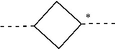
(b) A is selected from the group consisting of 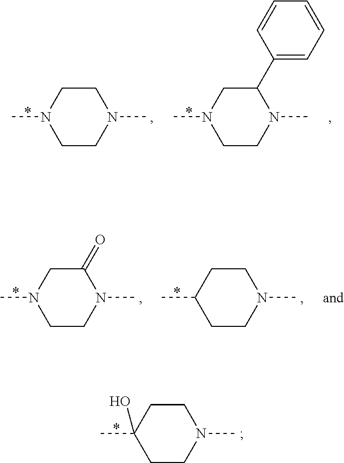
and
(c) B is selected from the group consisting of
methyl,

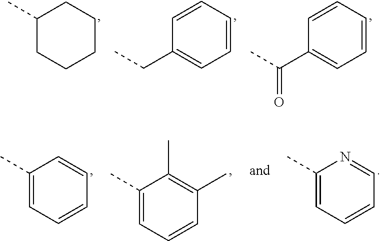

14-15. (canceled)

16. The compound of claim 1, wherein R^{3a} is selected from the group consisting of
methyl,

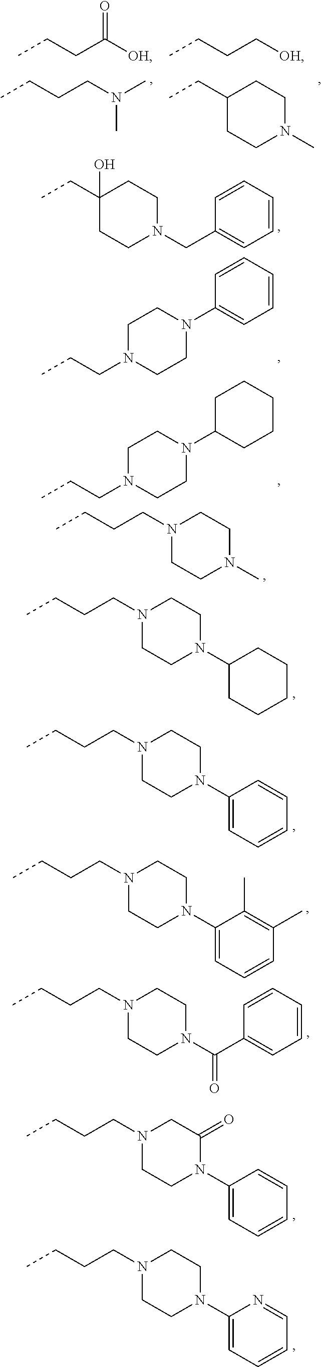

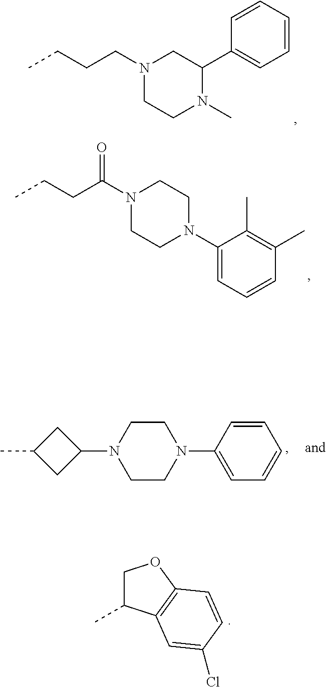

17. The compound of claim 1, wherein each occurrence of alkyl, cycloalkyl, alkylenyl, cycloalkylenyl, heterocycloalkylenyl, heterocycloalkyl, heteroalkyl, aralkyl, aryl, heteroaryl, is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂OR^{I}, N(R^{I})(R^{II}), C₁-C₆ haloalkoxy, C₃-C₈ halocycloalkoxy, aryl, heteroaryl, (C₁-C₆ alkylenyl)C(═O)N(R^{I})(R^{II}), (C₁-C₆ alkylenyl)C(═O)OR^{I}, O(C₁-C₃ alkylenyl)C(═O)OR^{II}, O(C₁-C₃ alkylenyl)C(═O)N(R^{I})(R^{II}), C(═O)R^{I}, C(═O)OR^{I}, OC(═O)R^{I}, OC(═O)OR^{I}, SR^{I}, S(═O)R^{I}, S(═O)₂R^{I}, S(═O)₂N(R^{I})(R^{II}), S(═O)₂NR^{I}C(═O)NHR^{II}, N(R^{I})S(═O)₂R^{II}, N(R^{I})C(═O)R^{II}, and C(═O)NR^{I}R^{II}, wherein R^{I} and R^{II} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

18. The compound of claim 1, which is selected from the group consisting of:
2-(3-iodo-4,5-dimethoxyphenyl)-1-((1S,3S)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-((1R,3S)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-((1S,3R)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-((1R,3R)-3-(methylcarbamoyl)cyclopentyl)-N-(3-(4-phenylpiperazin-1-yl)propyl)-1H-benzo[d]imidazole-6-carboxamide;
2-(3-iodo-4,5-dimethoxyphenyl)-1-(3-(methylcarbamoyl)cyclopentyl)-N-(3-(4-phenylpiperazin-1-yl)propyl)-1H-benzo[d]imidazole-6-carboxamide;
1-((1S,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1R,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1S,3R)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1R,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-(3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
N-(5-chloro-2,3-dihydrobenzofuran-3-yl)-1-((1S,3S)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-((1R,3S)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-((1S,3R)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-((1R,3R)-3-(methylcarbamoyl)cyclopentyl)-2-(3,4,5-trimethoxyphenyl)-1H-benzo[d]imidazole-6-carboxamide;
N-(5-chloro-2,3-dihydrobenzofuran-3-yl)-1-(3-(methylcarbamoyl)cyclopentyl)-2-(3,4,5-trimethoxyphenyl)-1H-benzo[d]imidazole-6-carboxamide;
N-methyl-1-((1S,3S)-3-(methylcarbamoyl)cyclopentyl)-2-(3,4,5-trimethoxyphenyl)-1H-benzo[d]imidazole-6-carboxamide;
N-methyl-1-((1R,3S)-3-(methylcarbamoyl)cyclopentyl)-2-(3,4,5-trimethoxyphenyl)-1H-benzo[d]imidazole-6-carboxamide;
N-methyl-1-((1S,3R)-3-(methylcarbamoyl)cyclopentyl)-2-(3,4,5-trimethoxyphenyl)-1H-benzo[d]imidazole-6-carboxamide;
N-methyl-1-((1R,3R)-3-(methylcarbamoyl)cyclopentyl)-2-(3,4,5-trimethoxyphenyl)-1H-benzo[d]imidazole-6-carboxamide;
N-methyl-1-(3-(methylcarbamoyl)cyclopentyl)-2-(3,4,5-trimethoxyphenyl)-1H-benzo[d]imidazole-6-carboxamide;
1-((1r,3r)-3-(methylcarbamoyl)cyclobutyl)-N-(3-(4-phenylpiperazin-1-yl)propyl)-2-(3,4,5-trimethoxyphenyl)-1H-benzo[d]imidazole-6-carboxamide;
1-((1r,3s)-3-(methylcarbamoyl)cyclobutyl)-N-(3-(4-phenylpiperazin-1-yl)propyl)-2-(3,4,5-trimethoxyphenyl)-1H-benzo[d]imidazole-6-carboxamide;
1-(3-(methylcarbamoyl)cyclobutyl)-N-(3-(4-phenylpiperazin-1-yl)propyl)-2-(3,4,5-trimethoxyphenyl)-1H-benzo[d]imidazole-6-carboxamide;
N-(3-(4-phenylpiperazin-1-yl)propyl)-2-(3,4,5-trimethoxyphenyl)-1H-benzo[d]imidazole-6-carboxamide;
(1r,3r)-N-methyl-3-(6-(4-phenylpiperazine-1-carbonyl)-2-(3,4,5-trimethoxyphenyl)-1H-benzo[d]imidazol-1-yl)cyclobutane-1-carboxamide;
(1r,3s)-N-methyl-3-(6-(4-phenylpiperazine-1-carbonyl)-2-(3,4,5-trimethoxyphenyl)-1H-benzo[d]imidazol-1-yl)cyclobutane-1-carboxamide;
N-methyl-3-(6-(4-phenylpiperazine-1-carbonyl)-2-(3,4,5-trimethoxyphenyl)-1H-benzo[d]imidazol-1-yl)cyclobutane-1-carboxamide;
1-((1r,3r)-3-(methylcarbamoyl)cyclobutyl)-N-(3-(4-methylpiperazin-1-yl)propyl)-2-(3,4,5-trimethoxyphenyl)-1H-benzo[d]imidazole-6-carboxamide;
1-((1r,3s)-3-(methylcarbamoyl)cyclobutyl)-N-(3-(4-methylpiperazin-1-yl)propyl)-2-(3,4,5-trimethoxyphenyl)-1H-benzo[d]imidazole-6-carboxamide;
1-(3-(methylcarbamoyl)cyclobutyl)-N-(3-(4-methylpiperazin-1-yl)propyl)-2-(3,4,5-trimethoxyphenyl)-1H-benzo[d]imidazole-6-carboxamide;
N-(3-(dimethylamino)propyl)-1-((1r,3r)-3-(methylcarbamoyl)cyclobutyl)-2-(3,4,5-trimethoxyphenyl)-1H-benzo[d]imidazole-6-carboxamide;
N-(3-(dimethylamino)propyl)-1-((1r,3s)-3-(methylcarbamoyl)cyclobutyl)-2-(3,4,5-trimethoxyphenyl)-1H-benzo[d]imidazole-6-carboxamide;
N-(3-(dimethylamino)propyl)-1-(3-(methylcarbamoyl)cyclobutyl)-2-(3,4,5-trimethoxyphenyl)-1H-benzo[d]imidazole-6-carboxamide;
1-((1r,3r)-3-(methylcarbamoyl)cyclobutyl)-N-((1-methylpiperidin-4-yl)methyl)-2-(3,4,5-trimethoxyphenyl)-1H-benzo[d]imidazole-6-carboxamide;
1-((1r,3s)-3-(methylcarbamoyl)cyclobutyl)-N-((1-methylpiperidin-4-yl)methyl)-2-(3,4,5-trimethoxyphenyl)-1H-benzo[d]imidazole-6-carboxamide;
1-(3-(methylcarbamoyl)cyclobutyl)-N-((1-methylpiperidin-4-yl)methyl)-2-(3,4,5-trimethoxyphenyl)-1H-benzo[d]imidazole-6-carboxamide;
2-(3-methoxyphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(3-methoxyphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(3-methoxyphenyl)-1-(3-(methylcarbamoyl)cyclobutyl)-N-(3-(4-phenylpiperazin-1-yl)propyl)-1H-benzo[d]imidazole-6-carboxamide;
2-(3,5-dimethoxyphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(3,5-dimethoxyphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(3,5-dimethoxyphenyl)-1-(3-(methylcarbamoyl)cyclobutyl)-N-(3-(4-phenylpiperazin-1-yl)propyl)-1H-benzo[d]imidazole-6-carboxamide;
1-cyclobutyl-N-(3-(4-phenylpiperazin-1-yl)propyl)-2-(3,4,5-trimethoxyphenyl)-1H-benzo[d]imidazole-6-carboxamide;
1-(4-(methylamino)-4-oxobutyl)-N-(3-(4-phenylpiperazin-1-yl)propyl)-2-(3,4,5-trimethoxyphenyl)-1H-benzo[d]imidazole-6-carboxamide;
1-((1r,3r)-3-(methylcarbamoyl)cyclobutyl)-2-phenyl-N-(3-(4-phenylpiperazin-1-yl)propyl)-1H-benzo[d]imidazole-6-carboxamide;
1-((1r,3s)-3-(methylcarbamoyl)cyclobutyl)-2-phenyl-N-(3-(4-phenylpiperazin-1-yl)propyl)-1H-benzo[d]imidazole-6-carboxamide;
1-(3-(methylcarbamoyl)cyclobutyl)-2-phenyl-N-(3-(4-phenylpiperazin-1-yl)propyl)-1H-benzo[d]imidazole-6-carboxamide;
2-(4-carbamoylphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(4-carbamoylphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(4-carbamoylphenyl)-1-(3-(methylcarbamoyl)cyclobutyl)-N-(3-(4-phenylpiperazin-1-yl)propyl)-1H-benzo[d]imidazole-6-carboxamide;
2-(3-fluoro-5-methoxyphenyl)-1-((1r,3r)-3-(methylcarbamoyl)cyclobutyl)-N-(3-(4-phenylpiperazin-1-yl)propyl)-1H-benzo[d]imidazole-6-carboxamide;
2-(3-fluoro-5-methoxyphenyl)-1-((1r,3s)-3-(methylcarbamoyl)cyclobutyl)-N-(3-(4-phenylpiperazin-1-yl)propyl)-1H-benzo[d]imidazole-6-carboxamide;
2-(3-fluoro-5-methoxyphenyl)-1-(3-(methylcarbamoyl)cyclobutyl)-N-(3-(4-phenylpiperazin-1-yl)propyl)-1H-benzo[d]imidazole-6-carboxamide;
N-(3-(4-(2,3-dimethylphenyl)piperazin-1-yl)propyl)-1-((1r,3r)-3-(methylcarbamoyl)cyclobutyl)-2-(3,4,5-trimethoxyphenyl)-1H-benzo[d]imidazole-6-carboxamide;
N-(3-(4-(2,3-dimethylphenyl)piperazin-1-yl)propyl)-1-((1r,3s)-3-(methylcarbamoyl)cyclobutyl)-2-(3,4,5-trimethoxyphenyl)-1H-benzo[d]imidazole-6-carboxamide;
N-(3-(4-(2,3-dimethylphenyl)piperazin-1-yl)propyl)-1-(3-(methylcarbamoyl)cyclobutyl)-2-(3,4,5-trimethoxyphenyl)-1H-benzo[d]imidazole-6-carboxamide;
1-cyclobutyl-N-(3-(4-(2,3-dimethylphenyl)piperazin-1-yl)propyl)-2-(3,4,5-trimethoxyphenyl)-1H-benzo[d]imidazole-6-carboxamide;
2-(2,5-dimethoxyphenyl)-N-(3-(4-(2,3-dimethylphenyl)piperazin-1-yl)propyl)-1-((1r,3r)-3-(methylcarbamoyl)cyclobutyl)-1H-benzo[d]imidazole-6-carboxamide;
2-(2,5-dimethoxyphenyl)-N-(3-(4-(2,3-dimethylphenyl)piperazin-1-yl)propyl)-1-((1r,3s)-3-(methylcarbamoyl)cyclobutyl)-1H-benzo[d]imidazole-6-carboxamide;
2-(2,5-dimethoxyphenyl)-N-(3-(4-(2,3-dimethylphenyl)piperazin-1-yl)propyl)-1-(3-(methylcarbamoyl)cyclobutyl)-1H-benzo[d]imidazole-6-carboxamide;
2-(3,4-dimethoxyphenyl)-N-(3-(4-(2,3-dimethylphenyl)piperazin-1-yl)propyl)-1-((1r,3r)-3-(methylcarbamoyl)cyclobutyl)-1H-benzo[d]imidazole-6-carboxamide;
2-(3,4-dimethoxyphenyl)-N-(3-(4-(2,3-dimethylphenyl)piperazin-1-yl)propyl)-1-((1r,3s)-3-(methylcarbamoyl)cyclobutyl)-1H-benzo[d]imidazole-6-carboxamide;
2-(3,4-dimethoxyphenyl)-N-(3-(4-(2,3-dimethylphenyl)piperazin-1-yl)propyl)-1-(3-(methylcarbamoyl)cyclobutyl)-1H-benzo[d]imidazole-6-carboxamide;
3-(1-cyclobutyl-2-(3,4,5-trimethoxyphenyl)-1H-benzo[d]imidazole-6-carboxamido)propanoic acid;
1-cyclobutyl-N-(3-hydroxypropyl)-2-(3,4,5-trimethoxyphenyl)-1H-benzo[d]imidazole-6-carboxamide;
1-cyclobutyl-N-(3-(4-(2,3-dimethylphenyl)piperazin-1-yl)-3-oxopropyl)-2-(3,4,5-trimethoxyphenyl)-1H-benzo[d]imidazole-6-carboxamide;
1-cyclopropyl-N-(3-(4-(2,3-dimethylphenyl)piperazin-1-yl)propyl)-2-(3,4,5-trimethoxyphenyl)-1H-benzo[d]imidazole-6-carboxamide;
1-cyclobutyl-2-(3,5-dimethoxyphenyl)-N-(3-(4-(2,3-dimethylphenyl)piperazin-1-yl)propyl)-1H-benzo[d]imidazole-6-carboxamide;
1-cyclobutyl-N-(3-(dimethylamino)propyl)-2-(3-fluoro-5-methoxyphenyl)-1H-benzo[d]imidazole-6-carboxamide;
1-cyclobutyl-2-(3,5-difluorophenyl)-N-(3-(dimethylamino)propyl)-1H-benzo[d]imidazole-6-carboxamide;
1-cyclobutyl-N-(3-(dimethylamino)propyl)-2-(pyridin-4-yl)-1H-benzo[d]imidazole-6-carboxamide;
1-cyclobutyl-N-(3-(dimethylamino)propyl)-2-(6-(4-methoxyphenyl)pyridin-2-yl)-1H-benzo[d]imidazole-6-carboxamide;
2-([1,1′-biphenyl]-3-yl)-1-cyclobutyl-N-(3-(dimethylamino)propyl)-1H-benzo[d]imidazole-6-carboxamide;
1-((1R,3R)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1S,3R)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1R,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-((1S,3S)-3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-(3-(methylcarbamoyl)cyclopentyl)-N-(3-(4-phenylpiperazin-1-yl)propyl)-2-(3,4,5-trimethoxyphenyl)-1H-benzo[d]imidazole-6-carboxamide;
1-cyclobutyl-N-(3-(dimethylamino)propyl)-2-(isoquinolin-4-yl)-1H-benzo[d]imidazole-6-carboxamide;
1-cyclobutyl-N-(3-(dimethylamino)propyl)-2-(quinolin-4-yl)-1H-benzo[d]imidazole-6-carboxamide;
1-cyclobutyl-N-(3-(dimethylamino)propyl)-2-(1H-pyrrolo[2,3-b]pyridin-3-yl)-1H-benzo[d]imidazole-6-carboxamide;
N-(3-(dimethylamino)propyl)-2-(3-fluoro-5-methoxyphenyl)-1-((1r,3r)-3-(methylcarbamoyl)cyclobutyl)-1H-benzo[d]imidazole-6-carboxamide;
N-(3-(dimethylamino)propyl)-2-(3-fluoro-5-methoxyphenyl)-1-((1r,3s)-3-(methylcarbamoyl)cyclobutyl)-1H-benzo[d]imidazole-6-carboxamide;
N-(3-(dimethylamino)propyl)-2-(3-fluoro-5-methoxyphenyl)-1-(3-(methylcarbamoyl)cyclobutyl)-1H-benzo[d]imidazole-6-carboxamide;
N-(3-(dimethylamino)propyl)-2-(4-fluoro-3-methoxyphenyl)-1-((1r,3r)-3-(methylcarbamoyl)cyclobutyl)-1H-benzo[d]imidazole-6-carboxamide;
N-(3-(dimethylamino)propyl)-2-(4-fluoro-3-methoxyphenyl)-1-((1r,3s)-3-(methylcarbamoyl)cyclobutyl)-1H-benzo[d]imidazole-6-carboxamide;
N-(3-(dimethylamino)propyl)-2-(4-fluoro-3-methoxyphenyl)-1-(3-(methylcarbamoyl)cyclobutyl)-1H-benzo[d]imidazole-6-carboxamide;
N-(3-(dimethylamino)propyl)-2-(3-methoxyphenyl)-1-((1r,3r)-3-(methylcarbamoyl)cyclobutyl)-1H-benzo[d]imidazole-6-carboxamide;
N-(3-(dimethylamino)propyl)-2-(3-methoxyphenyl)-1-((1r,3s)-3-(methylcarbamoyl)cyclobutyl)-1H-benzo[d]imidazole-6-carboxamide;
N-(3-(dimethylamino)propyl)-2-(3-methoxyphenyl)-1-(3-(methylcarbamoyl)cyclobutyl)-1H-benzo[d]imidazole-6-carboxamide;
2-(3,4-dimethoxyphenyl)-N-(3-(dimethylamino)propyl)-1-((1r,3r)-3-(methylcarbamoyl)cyclobutyl)-1H-benzo[d]imidazole-6-carboxamide;
2-(3,4-dimethoxyphenyl)-N-(3-(dimethylamino)propyl)-1-((1r,3s)-3-(methylcarbamoyl)cyclobutyl)-1H-benzo[d]imidazole-6-carboxamide;
2-(3,4-dimethoxyphenyl)-N-(3-(dimethylamino)propyl)-1-(3-(methylcarbamoyl)cyclobutyl)-1H-benzo[d]imidazole-6-carboxamide;
2-(3,5-dimethoxyphenyl)-N-(3-(dimethylamino)propyl)-1-((1r,3r)-3-(methylcarbamoyl)cyclobutyl)-1H-benzo[d]imidazole-6-carboxamide;
2-(3,5-dimethoxyphenyl)-N-(3-(dimethylamino)propyl)-1-((1r,3s)-3-(methylcarbamoyl)cyclobutyl)-1H-benzo[d]imidazole-6-carboxamide;
2-(3,5-dimethoxyphenyl)-N-(3-(dimethylamino)propyl)-1-(3-(methylcarbamoyl)cyclobutyl)-1H-benzo[d]imidazole-6-carboxamide;
2-(2,5-dimethoxyphenyl)-N-(3-(dimethylamino)propyl)-1-((1r,3r)-3-(methylcarbamoyl)cyclobutyl)-1H-benzo[d]imidazole-6-carboxamide;
2-(2,5-dimethoxyphenyl)-N-(3-(dimethylamino)propyl)-1-((1r,3s)-3-(methylcarbamoyl)cyclobutyl)-1H-benzo[d]imidazole-6-carboxamide;
2-(2,5-dimethoxyphenyl)-N-(3-(dimethylamino)propyl)-1-(3-(methylcarbamoyl)cyclobutyl)-1H-benzo[d]imidazole-6-carboxamide;
1-cyclobutyl-N-(2-(4-phenylpiperazin-1-yl)ethyl)-2-(3,4,5-trimethoxyphenyl)-1H-benzo[d]imidazole-6-carboxamide;
1-cyclobutyl-N-(3-(4-(pyridin-2-yl)piperazin-1-yl)propyl)-2-(3,4,5-trimethoxyphenyl)-1H-benzo[d]imidazole-6-carboxamide;
1-cyclobutyl-N-(3-(4-phenylpiperazin-1-yl)cyclobutyl)-2-(3,4,5-trimethoxyphenyl)-1H-benzo[d]imidazole-6-carboxamide;
1-cyclobutyl-N-(3-(dimethylamino)propyl)-2-(3,4,5-trimethoxyphenyl)-1H-benzo[d]imidazole-6-carboxamide;
1-cyclobutyl-N-(3-(3-oxo-4-phenylpiperazin-1-yl)propyl)-2-(3,4,5-trimethoxyphenyl)-1H-benzo[d]imidazole-6-carboxamide;
N-(3-(4-benzoylpiperazin-1-yl)propyl)-1-cyclobutyl-2-(3,4,5-trimethoxyphenyl)-1H-benzo[d]imidazole-6-carboxamide;
1-cyclobutyl-N-(3-(4-cyclohexylpiperazin-1-yl)propyl)-2-(3,4,5-trimethoxyphenyl)-1H-benzo[d]imidazole-6-carboxamide;
1-cyclobutyl-N-(3-(4-methyl-3-phenylpiperazin-1-yl)propyl)-2-(3,4,5-trimethoxyphenyl)-1H-benzo[d]imidazole-6-carboxamide;
1-cyclobutyl-N-(2-(4-cyclohexylpiperazin-1-yl)ethyl)-2-(3,4,5-trimethoxyphenyl)-1H-benzo[d]imidazole-6-carboxamide; and
N-((1-benzyl-4-hydroxypiperidin-4-yl)methyl)-1-cyclobutyl-2-(3,4,5-trimethoxyphenyl)-1H-benzo[d]imidazole-6-carboxamide.

19. An antibody-drug conjugate (ADC) comprising at least one compound of claim 1 and an antibody, wherein the compound is covalently conjugated to the antibody.

20. The ADC of claim 19, wherein at least one of the following applies:
(a) the antibody specifically binds to AMHR2; and
(b) the ADC is suitable for delivery of the at least one compound to at least one cell type selected from the group consisting of ovarian granulosa cells and endometrioma cells.

21. (canceled)

22. A pharmaceutical composition comprising at least one compound of claim 1 and a pharmaceutically acceptable carrier, optionally wherein the pharmaceutical composition further comprises at least one additional therapeutically effective agent.

23. (canceled)

24. A method of treating, preventing, or ameliorating a disease or disorder in a subject in need thereof, the method comprising administering to the subject a therapeutically effective amount of at least one compound of claim 1, or a pharmaceutical composition thereof.

25. The method claim 24, wherein at least one of the following applies:
(a) the disease or disorder is characterized by one or more ACVR1 activating mutations or overactive ACVR1, optionally wherein the disease or disorder is at least one selected from the group consisting of fibrodysplasia ossificans progressiva (FOP), atherosclerosis, anemia, iron deficiency, tumor angiogenesis, tumor vasculogenesis, and cancer, and optionally wherein the cancer is at least one selected from the group consisting of diffuse intrinsic pontine glioma (DIPG), ependymoma, breast cancer, endometrial cancer, and pancreatic cancer; and
(b) the subject is further administered at least one additional therapeutic agent.

26-28. (canceled)

29. A method of inhibiting or reducing activity of ACVR1 or ACVRL1 in a subject in need thereof, the method comprising administering to the subject a therapeutically effective amount of at least one compound of claim 1, or a pharmaceutical composition thereof.

30. The method of claim 29, wherein at least one of the following applies:
(a) the ACVR1 is ACVR1^{WT} or an ACVR1 mutant comprising one or more activating mutations; and
(b) the ACVRL1 is ACVRL1^{WT} or an ACVRL1 mutant comprising one or more activating mutations.

31-32. (canceled)
